(19) United States
(12) Patent Application Publication
(10) Pub. No.: US 2026/0091099 A1
(43) Pub. Date: Apr. 2, 2026

(54) METHODS FOR PREVENTING THE DEVELOPMENT OF A GASTRIC DISEASE ASSOCIATED WITH HELICOBACTER PYLORI INFECTION WITHOUT ERADICATING THE INFECTION AND FOR THE ASSESSMENT OF THE RISK OF DEVELOPING SUCH DISEASE

(71) Applicants: Thomas BORÉN, Umeå (SE); Lennart HAMMARSTRÖM, Huddinge (SE)

(72) Inventors: Thomas BORÉN, Umeå (SE); Lennart HAMMARSTRÖM, Huddinge (SE)

(21) Appl. No.: 19/352,770

(22) Filed: Oct. 8, 2025

Related U.S. Application Data
(63) Continuation of application No. PCT/EP2024/060099, filed on Apr. 12, 2024.

(30) Foreign Application Priority Data
Apr. 12, 2023 (SE) 2350423-6

(51) Int. Cl. A61K 39/02; A61K 39/00; A61P 37/04

(52) U.S. Cl. CPC A61K 39/105; A61P 37/04; A61K 2039/55505; A61K 2039/55544; A61K 2039/585; A61K 2039/828

(57) ABSTRACT

Recombinant blood group antigen-binding adhesin (BabA) can be used in a composition to raise humoral immune response i.e. antibodies, that target conserved structural epitopes in the fucosylated blood group antigen Lewis b (Leb) antigen binding domain in the blood group antigen-binding adhesin (BabA). Such broadly Leb-blocking antibodies can reduce the gastric mucosal attachment of Helicobacter pylori (H. pylori) to human gastric mucosa. This approach can be used for preventing and/or alleviating gastric disease in a subject diagnosed with H. pylori infection, said gastric disease chosen from gastroesophageal reflux disease, chronic active gastritis, peptic ulcer diseases, gastric ulcer disease, gastric cancer gastritis and gastric cancer, in particular gastric cancer. It also becomes possible to identify subjects that carry BabA positive H. pylori infections and subjects that are at risk of developing severe gastric disease, such as peptic ulcer diseases, gastric ulcer disease, gastritis with metaplasia and gastric cancer.

### STATEMENT OF PRIORITY

This application is a continuation of International Application No. PCT/EP2024/060099, filed on Apr. 12, 2024, which claims priority from Swedish Application No. 2350423-6, filed on Apr. 12, 2023, the entire contents of each of which is incorporated herein by reference.

### TECHNICAL FIELD

The present disclosure relates to the prevention and treatment of gastric disease associated with Helicobacter pylori (H. pylori) infection without eradicating the infection, and in particular the prevention and treatment of gastric cancers associated with an ongoing infection. The disclosure relates to methods of raising broadly blocking antibodies (bbAbs) reducing H. pylori binding and attachment to the gastric mucosa, which has now been shown to protect against gastric disease, in particular gastric cancer. The disclosure also relates to vaccines and vaccine compositions and their use in such methods. The disclosure further presents a diagnostic approach to identify subjects at risk of developing gastric disease, and to identify and/or classify subjects that would benefit from a treatment reducing H. pylori binding and attachment to the gastric mucosa, as well as diagnostic/prognostic kits for performing said classification.

### BACKGROUND

The gastric onco-pathogen H. pylori lives in the stomach lining, referred to as the gastric epithelium. Close to the epithelial cells, the viscous mucus layer provides a bicarbonate buffer zone that protects the epithelial cell lining from the acidic gastric juice present in the stomach lumen just a third of a millimetre away. However, as the distance from the cell lining increases, the bicarbonate buffering fades to form a pH gradient that extends about half-way up the mucus layer. At this “altitude”, the impact of the very acidic gastric juice returns the pH to pH 1-2. The gastric juice is acidified by hydrochloric acid, which of course is too acidic for most microbes, H. pylori included. In this fascinating environment, H. pylori has adapted its life in the stomach to make life-long use of both the epithelial cell lining and the mucus layer pH gradient (Bugaytsova et al., 2017 and Schreiber et al., 2004).

The more virulent H. pylori isolates bind the ABO blood group antigens on the epithelial cell surfaces for firm bacterial attachment. In this prime location, H. pylori has full access to the necessary nutrients and iron leaching from the mucosal cells. However, pros come with cons, and the tight attachment exposes H. pylori to the human immune system. As a result, the H. pylori infection is a major target of the humoral immune system, and the majority of carriers produce ELISA titers against H. pylori antigens. Hence, the H. pylori infections are exposed to high levels of antibodies that disseminate into the gastrointestinal tissues from the systemic circulation as well as from antibody-producing cells in both the stomach and gut lining. These antibodies bind to a plethora of H. pylori antigens such as the urease enzyme, LPS, heat-shock proteins, and the CagA effector protein.

Although the human immune system mobilizes the best possible defence, the antibodies against H. pylori never manage to entirely prevent the H. pylori infection, which instead establish itself through adaptation and persists over the lifespan. The persistent infection is the cause of the chronic mucosal inflammation that in many millions of cases annually results in gastric disease, although of different degree of severity, ranging from mild and non-atrophic gastritis to gastric cancer.

Chronic life-long infection by H. pylori is the main cause of chronic active gastritis, which can lead to severe clinical conditions including gastric and duodenal ulcer disease and gastric cancer. All these diseases except gastric cancer have become at least temporarily curable by H. pylori eradication; however, gastric cancer remains a major threat to human life with about 1.2 million new cases every year with a poor survival rate, with approx. 25 million deaths since the year 2000. About 5-10% of H. pylori-infected individuals experience duodenal ulcer disease, which, although curable, can perforate causing bleeding with a high mortality. Subjects with ulcers or gastric cancer typically carry the more virulent “triple-positive infections” characterized by H. pylori bearing the CagA onco-effector protein, the VacA toxin, and the Blood group Antigen Binding (BabA) attachment protein (adhesin).

BabA is a member of the H. pylori outer membrane protein family and binds to the ABO/Lewis b (Leb) blood group antigens expressed in the gastro-intestinal lining, including the gastric epithelium, see e.g., Boren et al., 1993. BabA exhibits rapid and diversifying selection through amino acid substitutions in its Leb-carbohydrate binding domain (CBD) that adapts its binding preferences for different human populations and ABO blood group phenotypes. See e.g. Aspholm-Hurtig et al., 2004.

The rapid mutation and selection in BabA also allow for adaptation to the individual and optimal binding to the different locations and microenvironments in the stomach, including adaptation to shifts in acidity caused by diseases such as peptic ulcer disease and gastric cancer and adaptation by shifts in its binding affinity, see e.g. Bugaytsova et al., 2017. As a consequence, BabA exhibits extensive global diversity where nearly every infected individual will harbour a strain of H. pylori possessing a unique BabA sequence. H. pylori infections induce high antibody titers against several dominant antigens, such as its urease enzyme, but these antibodies fail to clear the infection. Hence, H. pylori infections instead become persistent and life-long. By contrast, most H. pylori outer membrane proteins, including BabA, are reported to be weakly antigenic. Immunoglobulin G is induced by gastro-intestinal (GI) pathogens also in the gut and by H. pylori virulence factors in both the gastric antrum and the corpus mucosa (Kamada et al., 2015).

The immune responses however fail to eradicate the infection and also fail to protect against reinfection after antibiotic treatment. The chronic outcome is understandable considering that H. pylori, over the millennia of human infections, has established multiple mechanisms to evade the human immune responses through fast and confluent adaptation by high mutation rates and antigenic variation combined with dysregulation of T-cell responses (For a comprehensive review article, see Salama et al., 2013). The many escape routes are reflected in both the shortcomings of conventional H. pylori vaccine development (Sutton and Boag, 2019) and the high re-infection rate in many parts of the world after eradication of infection with antibiotics. Consequently, the elusive and extremely adaptable “character” of this bacteria, together with its global spread, makes it very challenging target for both therapy and diagnosis. Existing approaches to this are exemplified below:

WO 9747646A1 discloses a test kit comprising a monospecific antiserum that recognizes a BabA antigen and comprises an immunoglobulin that binds said BabA antigen. The protein sequence of BabA, and the DNA sequences for the BabA and BabB genes are disclosed. The adhesin and/or DNA is said to be useful for diagnosis and therapy and/or prophylaxis directed against H. pylori induced infections, e.g., gastritis and acid peptic disease, i.e. active or passive vaccination.

US6410719B1 further discloses isolated recombinant DNA from H. pylori blood group antigen binding (BAB) adhesins, BabA and BabB, respectively.

US8025880B2 discloses a purified and isolated specific variable antibody binding region exhibiting specific activity to the native and conformational dependent BabA antigen expressed by H. pylori wherein the antibody is a fully human immunoglobulin IgG1 antibody comprising an Abba3 variable heavy chain region with a specific amino acid sequence and a variable light chain region with another amino acid sequence, and said antibody neutralizes H. pylori by specifically binding to the native BabA protein.

WO2008105740A1 and US2008213224A1 disclose a method for identification and selection of human single chain fragment variable (scFv) antibody fragments with specificity for the BabA protein, in particular a scFv clone that competes for binding with the Lewis b antigen (Leb).

WO02066502A1 and US2004126811A1 disclose the H. pylori sialic acid binding Adhesin (SabA) and SabA-gene, antibodies against sialyl-Lewis x antigen-binding protein, AbbA3, and the use of SabA as a medicament or diagnostic antigen, and for determining the presence of sialyl-Lewis x antigen-binding H. pylori bacteria in a biological sample.

WO0056343A1 and e.g. EP1169044B1 present the use of a fucosylated sialylated N-acetyl lactosamine structure and/or a sialyl-Lewis antigen carbohydrate structure for the preparation of a pharmaceutical composition for the treatment or prophylaxis in humans of infections by H. pylori of the human gastrointestinal mucosa.

The global high prevalence of H. pylori infection, the high susceptibility to reinfection after antibiotic treatment, and the alarmingly fast development of antibiotic resistance and the strong link to gastric disease and in particular gastric cancer makes it an urgent task to find new approaches to address this issue. Despite decades of research and development, there is still a need for tools for assessing the risk of sequelae and for dealing with the fact that H. pylori is so resistant to eradication.

### SUMMARY

Therefore, the present inventors commissioned the testing of approx. 1000 subject sera and identified broadly blocking IgG Abs (bbAbs). It was found that subjects with duodenal ulcer disease (DU) exhibited low titers of bbAbs. The result indicate that the IgG-mediated blocking activity is protective throughout the GI tract and forms a basis for the inventions defined in the attached claims and embodiments thereof. To identify the bbAb binding epitopes in BabA, the present inventors cloned a human antibody, Anti-BabA, denoted as ABbA, from healthy H. pylori carriers and co-crystalized it with BabA. The co-crystal structure showed that ABbA and Leb bind to the same residues in the BabA CBD. Thus, ABbA is resistant to microbial antigen variation and instead performs fulminant glycan mimicry to block and compete with BabA-mediated Leb binding to reduce mucosal attachment of H. pylori. The present results indicate that the host's blocking antibody response has co-evolved with H. pylori as a way of tolerating chronic infection and reducing the risk for overt gastric disease by mitigating mucosal inflammation. It is therefore postulated that the bbAbs reported here offer translational applications in treatment and predictive diagnostics for individuals at risk for severe gastric disease.

The present inventors have identified broadly blocking antibodies (bbAbs) that target conserved structural epitopes in the Leb-binding domain in the otherwise highly polymorphic BabA adhesin. The inventors have shown that vaccination with H. pylori attachment proteins effectively protects against gastric diseases associated with H-pylori, such as gastric cancer, and that this effect is achieved without eradicating the H. pylori infection, and consequently here make available the following inventions defined herein and in the attached claims.

Consequently, a first aspect of the present disclosure relates to a pharmaceutical composition comprising an immunogenically effective amount of an antigen for use in a method of preventing or treating gastric disease in a subject diagnosed with H. pylori infection, wherein said antigen is capable of inducing an antibody response sufficient to reduce mucosal attachment of H. pylori without eradicating the H. pylori infection; said antibody response specifically targets a conserved structural epitope in the Carbohydrate Binding Domain (CBD) of the ABO/Leb-Blood group Antigen-Binding Adhesin (BabA) of H. pylori; and said gastric disease is chosen from gastroesophageal reflux disease (GERD), chronic active gastritis (GA), peptic ulcer disease (DU), gastric ulcer disease, and gastric cancer.

According to an embodiment of said first aspect, the disease is gastric cancer, and wherein preventing gastric cancer is interpreted as the reduction of the incidence of gastric cancer in a population infected with H. pylori or susceptible to being infected by H. pylori, compared to the incidence in a control population not receiving said composition.

According to another embodiment of said first aspect and any embodiment thereof, the antigen is a recombinant modified BabA with an amino acid sequence having at least 80% identity, such as at least 85% identity, such as at least 90% identity, or at least 95% identity with the sequence defined by SEQ ID NO. 2 with the proviso that a continuous sequence of at least four positive amino acids is included in said sequence, preferably at the C-terminal of said sequence.

According to a preferred embodiment, said continuous sequence of at least four positive amino acid molecules is a sequence of at least four lysine molecules.

According to an alternative embodiment, said continuous sequence of at least four positive amino acid molecules is a sequence of at least four arginine molecules.

According to another, more preferred embodiment, said continuous sequence of at least four positive amino acid molecules is a sequence of at least six lysine molecules.

According to an embodiment of the first aspect, freely combinable with any of the above embodiments, the composition further comprises another antigen chosen from variants of BabA having four disulfide bonds whereof the second disulfide bond spans seven amino acids between two adjacent cystein molecules.

According to an embodiment of the first aspect, freely combinable with any of the above embodiments, the composition further comprises a paralogue BabB or a variant thereof having at least 80% identity with the sequence defined by SEQ ID NO. 3 or SEQ ID NO. 4, with the proviso that in variants of SEQ ID NO 4, a continuous sequence of at least four positive amino acids is included in said sequences, preferably at the C-terminal of said sequence.

According to a preferred embodiment of said first aspect, and freely combinable with any of the above, the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

A composition according to the first aspect or any embodiment thereof is preferably formulated as a vaccine comprising a pharmaceutically acceptable carrier and an adjuvant chosen from mucosal or systemic adjuvants that preferentially activate the humoral immune responses with induction of protective antibodies.

According to an embodiment of the above, said adjuvant is an aluminium-based adjuvant chosen from aluminum hydroxide, aluminum phosphate, and aluminum hydroxyphosphate sulfate.

According to an alternative embodiment, said adjuvant is a cholera toxin-derived adjuvant, more preferably CTA1-DD.

A second aspect of the present disclosure relates to a method for preventing, or alleviating or treating a gastric disease in a subject diagnosed with H. pylori infection, comprising administering to the subject an effective amount of a composition containing an antigen wherein the administration of said antigen results in the induction of an antibody response that targets a conserved structural epitope in the Carbohydrate Binding Domain (CBD) of the ABO/Leb-Blood group Antigen-Binding Adhesin (BabA), sufficient to reduce mucosal attachment of H. pylori without eradicating the H. pylori infection in said subject, and wherein said gastric disease is chosen from gastroesophageal reflux disease (GERD), chronic active gastritis (GA), peptic ulcer disease (DU), gastric ulcer disease, and gastric cancer.

According to an embodiment of said second aspect, the gastric disease is gastric cancer and wherein preventing gastric cancer is interpreted the reduction of the incidence of gastric cancer in a population infected with H. pylori or susceptible to being infected by H. pylori, compared to the incidence in a control population not receiving said composition.

According to another embodiment, freely combinable with the above, the antigen is a recombinant modified BabA with an amino acid sequence having at least 80% identity, such as at least 85% identity, such as at least 90% identity, or at least 95% identity with the sequence defined by SEQ ID NO. 2 with the proviso that a continuous sequence of at least four positive amino acids is included in said sequence, preferably at the C-terminal of said sequence.

According to a preferred embodiment, said continuous sequence of at least four positive amino acid molecules is a sequence of at least four lysine molecules.

According to another embodiment, said continuous sequence of at least four positive amino acid molecules is a sequence of at least four arginine molecules.

According to another, more preferred embodiment, said continuous sequence of at least four positive amino acid molecules is a sequence of at least six lysine molecules.

According to an embodiment of the first aspect, freely combinable with any of the above embodiments, the method further comprises administering another antigen chosen from variants of BabA having four disulfide bonds whereof the second disulfide bond spans seven amino acids between two adjacent cystein molecules.

According to a further embodiment, freely combinable with any of the above embodiments, the method further comprises administering a paralogue BabB or a variant thereof having at least 80% identity with the sequence defined by SEQ ID NO. 3 or SEQ ID NO. 4, with the proviso that, for variants of SEQ ID NO. 4, a continuous sequence of at least four positive amino acids is included in said sequences, preferably at the C-terminal of said sequence.

According to a preferred embodiment of said second aspect, and freely combinable with any of the above embodiments, the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

According to a preferred embodiment of said method, the composition is formulated as a vaccine comprising a pharmaceutically acceptable carrier and an adjuvant chosen from mucosal or systemic adjuvants that preferentially activate the humoral immune responses with induction of protective antibodies.

According to an embodiment of the second aspect, said adjuvant is an aluminium-based adjuvant chosen from aluminum hydroxide, aluminum phosphate, and aluminum hydroxyphosphate sulfate.

According to an alternative embodiment, said adjuvant is a cholera toxin-derived adjuvant, more preferably CTA1-DD.

A third aspect of the present disclosure relates to an in vitro method for assessing the risk of developing gastric disease in a subject infected with H. pylori, said method comprising the steps of
providing a sample taken from said subject;
mixing the sample with a labelled receptor conjugate, ABO/Leb (Leb) conjugate, forming a mixture of sample and conjugate;
incubating said mixture with suspension of a H. pylori bacterial isolate or recombinant BabA;
determining the presence and/or amount of Leb vs the broadly blocking antibodies (bbAbs from the sample) bound to the H. pylori bacterial isolate or recombinant BabA; and
determining the 50% inhibition titer (IT50) of Leb binding in said sample, ie the titer (IT50) of the sample that can reduce the ABO/Leb binding to the H. pylori bacterial isolate or recombinant BabA with 50%,
   wherein an IT50 value below 50 in the sample is taken as an indication of an increased risk of developing a gastric disease.

According to an embodiment of said third aspect, the sample is a sample chosen from plasma, serum, saliva, urine, or gastric fluid.

According to a preferred embodiment, an IT50 value below 30 in the serum sample is taken as an indication of an increased risk for developing a gastric disease.

A fourth aspect of the present disclosure relates to an in vitro method for classifying a subject with an H. pylori infection that would benefit from treatment with a composition as defined in any one of the above aspects and embodiments, or the method defined in any one of the above aspects and embodiments, said in vitro method comprising the steps of:
providing a sample taken from the subject;
mixing the sample with a labelled Lewis B (Leb) conjugate, ABO/Leb (Leb) conjugate, forming a mixture of sample and conjugate;
incubating said mixture with suspension of a H. pylori bacterial isolate or recombinant BabA;
determining the presence and/or amount of Leb vs the broadly blocking antibodies (bbAbs from the sample) bound to the H. pylori bacterial isolate or recombinant BabA; and determining the 50% inhibition titer (IT50) of Leb binding in said sample, i.e. the titer (IT50) of the sample that can reduce the ABO/Leb binding to the H. pylori bacterial isolate or recombinant BabA with 50%;
   wherein an IT50 value above background in the sample, where background level is determined with human H. pylori ELISA negative serum samples, is taken as an indication that said subject would benefit of a treatment with a composition as defined in any one of the claims 1-12 or the method according to claims 13-24, or wherein the subject would benefit from treatment with antibiotics.

According to an embodiment of the above fourth aspect, the sample is a sample chosen from plasma, serum, saliva, urine, or gastric fluid.

A positive IT50 signal is defined as an inhibition titer where the individual serum antibodies can reduce the signal of the labelled Leb conjugate bound to the suspension of a H. pylori bacterial isolate. A positive signal is an inhibition titer above background level and is defined as an inhibition titer that is above the titer that is obtained by use of pooled serum samples from one or preferable several ELISA negative, i.e., H. pylori negative individuals. Such sera are used to define the back-ground level (i.e., IT50 negative sera) because individuals who do not carry H. pylori infection and have never carried H. pylori infection, demonstrate no immune-response with IT50 inhibition titers of blocking antibodies against Leb-binding.

When classifying a subject with an H. pylori infection that would benefit from treatment, it is more important that the method is sensitive, so here a low-affinity Leb-binding isolate is used to achieve high sensitivity in identifying such subjects. By use of a low-affinity Leb binding isolate, the serum antibodies will out-compete the Leb binding more efficiently and hence produce an IT50 signal also in individuals with low IT50 levels. Thus, the method for detection of H. pylori infection will be much more sensitive using the use of a low-affinity Leb binding isolate.

A fifth aspect of the present disclosure relates to a kit for performing the method according to anu one of the third or fourth aspect or any embodiments thereof, wherein said kit comprises the reagents for performing the determination of IT50 for example by an Enzyme-Linked Immunosorbent Assay (ELISA), including a microtiter plate having wells wherein said wells are coated with recombinant BabA (or H. pylori bacterial isolate). Other immunoassay methods can also be used, such as but not limited to particle assisted immune assay methods, such as turbidimetric or nephelometric assays.

Consequently, according to an embodiment of the above aspect, said kit comprises the reagents for performing a turbidimetric determination of IT50, including for example a suspension of nanobeads with recombinant BabA immobilized to said nanobeads.

Such kit can be a kit for implementing any one of the methods as an enzyme-linked immunosorbent assay (ELISA), a radioimmunoassay (RIA), a lateral flow immunoassay (LFIA) etc. A person skilled in the art is well aware of the basic steps and elements of each assay type and can modify the assay and/or kit for performing the methods disclosed herein. In an ELISA, the antigen, recombinant BabA protein, is bound to the surface of a solid support, such as ELISA wells or plates, sample is added to said wells, and the broadly blocking antibodies (bbAbs) in the sample attach to the antigen. A secondary labelled antibody conjugate, capable of binding to the bbAbs, can then be added in order to allow quantification of the bbAbs in the sample. Alternatively, a Leb-conjugate that is with colorimetric labelled or fluorochrome labelled (or isotope labelled) can be used in the ELISA. The individual/subject serum broadly blocking antibodies (bbAbs) will then compete with the Leb conjugate in binding and will reduce the Leb conjugate colour signal. Such reduction in signal can be detected (by person skilled in the art) and the reduction in signal hence tells that the Individual carry bbAb because of H. pylori infection.

In RIA, the analyte, here the bbAbs, compete with radio-labelled antigen, e.g., Lewis B (leb) conjugate for binding to antibodies immobilized to a solid support.

In a LFIA device for detecting the presence or quantity of broadly blocking antibodies (bbAbs) against recombinant BabA residing in a test sample, the LFIA device comprises a porous membrane in liquid communication with a conjugate pad and a wicking pad:
said conjugate pad located upstream from a detection zone, said conjugate pad having detection probes with detectable markers and specific binding members for the analyte, and;
said detection zone having an immobilized first capture reagent, said first capture reagent being configured to bind to at least a portion of said analyte and analyte-conjugate complexes to generate a detection signal having an intensity;
a control zone located downstream from said detection zone, wherein a second capture reagent is immobilized within said control zone, said second capture reagent being configured to bind to said conjugate or conjugate-analyte complexes;
   wherein a sample containing said analyte is deposited on said conjugate pad and said sample moves toward said control zone, and wherein said analyte is a broadly blocking antibody (bbAb) against a recombinant BabA protein having an amino acid sequence with at least 80% homology with the sequence of SEQ ID NO. 1.

A sixth aspect of the present disclosure relates to an mRNA composition for use in preventing or treating a gastric disease in a subject diagnosed with H. pylori infection, wherein the mRNA encodes an antigen encapsulated in lipid nanoparticles, said antigen capable of inducing an antibody response sufficient to reduce mucosal attachment of H. pylori without eradicating the H. pylori infection; said antibody response specifically targeting a conserved structural epitope in the Carbohydrate Binding Domain (CBD) of the ABO/Leb-Blood group Antigen-Binding Adhesin (BabA) of H. pylori.

Associated therewith is a seventh aspect, which relates to a method for preventing a gastric disease in a subject diagnosed with H. pylori infection by eliciting an immune response in a mammal, comprising the steps of:
preparing a messenger RNA (mRNA) sequence encoding an antigen capable of inducing an antibody response sufficient to reduce mucosal attachment of H. pylori without eradicating the H. pylori infection; said antibody response specifically targeting a conserved structural epitope in the Carbohydrate Binding Domain (CBD) of the ABO/Leb-Blood group Antigen-Binding Adhesin (BabA) of H. pylori;
encapsulating said mRNA sequence in a lipid nanoparticle carrier to form an mRNA vaccine composition;
administering a therapeutically effective amount of said mRNA vaccine composition to the subject, wherein the lipid nanoparticle carrier facilitates delivery of the mRNA into the cells of the mammal;
expressing the antigen from the mRNA within the cells, thereby reducing mucosal attachment of H. pylori without eradicating the H. pylori infection in said subject.

According to an embodiment of any of the above aspects, the antigen is a BabA protein with an amino acid sequence having at least about 80% homology, such as at least 85% homology, such as at least 90% homology, or at least 95% homology with the sequence of SEQ ID NO. 1. The considerable variation or relatively low degree of homology (at least about 80%) is motivated by the fact that BabA is an unusual protein which exhibits considerable amino acid variation due to mutations. When investigating samples from geographically diverse populations, it has been found that the sequences can differ about 20%, such as 19%, 18%, 17%, 16%, 15%, 14%, 13%, 12%, 11%, 10%, 9%, 8%, 7%, 6%, 5%, 4%, 3%, 2% or 1% and still exhibit strong binding and pathogenicity.

According to an embodiment, said BabA protein is isolated and purified from natural sources, but according to a preferred embodiment, said BabA protein is recombinant BabA.

According to another aspect, the composition defined in any of the paragraphs above further comprises another antigen chosen from geographical variants of BabA. As noted above, BabA is an unusual protein which exhibits considerable amino acid variation due to mutations, and where geographically separated populations exhibit highly varying versions of the protein. It has been found that the sequences can differ about 20%, such as 19%, 18%, 17%, 16%, 15%, 14%, 13%, 12%, 11%, 10%, 9%, 8%, 7%, 6%, 5%, 4%, 3%, 2% or 1% and still exhibit strong binding and pathogenicity.

According to an embodiment of said aspect, the composition comprises the paralogue BabB. Such paralogue may have at least 80% homology, such as at least 85% homology, such as at least 90% homology, or at least 95% homology with the sequence of SEQ ID NO. 3 or SEQ ID NO. 4, with the proviso that, in variants of SEQ ID NO. 4, a continuous sequence of at least four positive amino acids is included in said sequences, preferably at the C-terminal of said sequence.

A specific aspect relates to methods for preventing, alleviating and/or treating a gastric condition or disease caused by or at least associated with H. pylori infection, such as gastroesophageal reflux disease (GERD), chronic active gastritis (GA), peptic ulcer disease (DU), gastric ulcer disease, and in particular gastric cancer, said methods comprising administering an immunogenically effective amount of an antigen to said subject, said antigen effective to raise an antibody that targets a conserved structural epitope in the fucosylated blood group antigen Lewis b (Leb) binding domain in the blood group antigen-binding adhesin (BabA).

Further aspects and embodiments will be apparent to a skilled person from a closer study of the below drawings, detailed description and examples.

### BRIEF DESCRIPTION OF THE DRAWINGS

The invention is now described, by way of examples, with reference to the accompanying drawings:

FIG. 1. High global prevalence of serum inhibition of Leb binding

Panel (A) shows the inhibition of in vitro binding to human gastric mucosa by H. pylori from six individual serum samples Inhibition was quantified (by ImageJ) of attached J166 bacterial cells treated with 1:10 or 1:50 dilutions of the individual serum samples. Individuals 1-3 demonstrated 100%, 100% and 39% inhibition of attachment and IT50s of 267, 70, and 58, respectively. The sera samples from individuals 4-6 did not block H. pylori binding and did not reduce Leb-binding to 50%. Hence, their IT50s were Non-Detected (ND).

Panel (B) The individual sera IT50 was determined by incubation of H. pylori J166 with ¹²⁵I-Leb conjugate in a dilution series of serum sample. The dashed vertical lines show the serum dilution when Leb binding was reduced by 50%, i.e., the serum dilution that equals the IT50. Serum from individuals 4-6 did not produce a 50% reduction (Non-Detected (ND) IT50)).

Panel (C) All the 38 individual sera from KI, Sweden. demonstrated IT50s, so to test a larger group of non-symptomatic individuals, we tested the IT50s of 322 individuals from the Swedish Kalixanda study. The sera were all tested by strain 17875/Leb with a median IT50=41 and a mean IT50=89. The J166 strain was more sensitive because of its lower Leb binding affinity and identified 84% of the sera as IT50 positive (FIG. 3, Panel A).

Panel (D) The IT50s of 141 ELISA-positive individuals from the USA tested with strain 17875/Leb with a median IT50=39 and a mean IT50=80, where 83 (59%) sera samples were positive for Leb inhibition by strain J166.

(Panel (E) The IT50s of 200 ELISA-positive individuals from Mexico City, Mexico, tested with strain 17875/Leb with a median IT50=50 and mean IT50=115, where 146 sera samples (72%) were positive for Leb inhibition by strain J166

Panel (F) The CagA status by ELISA among 317 out of 322 Kalixanda individuals (5 individuals were not tested for CagA) exhibited strong correlation with IT50, with a median IT50=11 for CagA-negative individuals and a median IT50=40 for CagA-positive individuals. *** p<0.001.

Panel (G) The median IT50s of the 322 Kalixanda individuals were identical between all the men and the women but increased with higher age in the four age groups (41-80 years) (Spearman p<0.001). The IT50s were similar in the four age groups for women and men, except for the 31-40-years group (Wilcoxon, p=0.03). The IT50s and H. pylori ELISAs of two individuals were followed (by biobank samples) over 26 and 18 years, respectively. The IT50s increased with age during the ˜50-year observation period in contrast to the almost constant ELISA titers.

Panel (H) The children's IT50s tested by strain 17875/Leb varied similarly to adults by 100-1000-fold.

In panel (I) the serum sample from individual 1 (bar 1) was applied to a Protein G column and the IgG was desorbed together with all of IT50 (bar 2). The IT50 was all recovered in the eluted IgG (bar 3).

FIG. 2. Broadly blocking human serum Abs protects against overt gastric disease

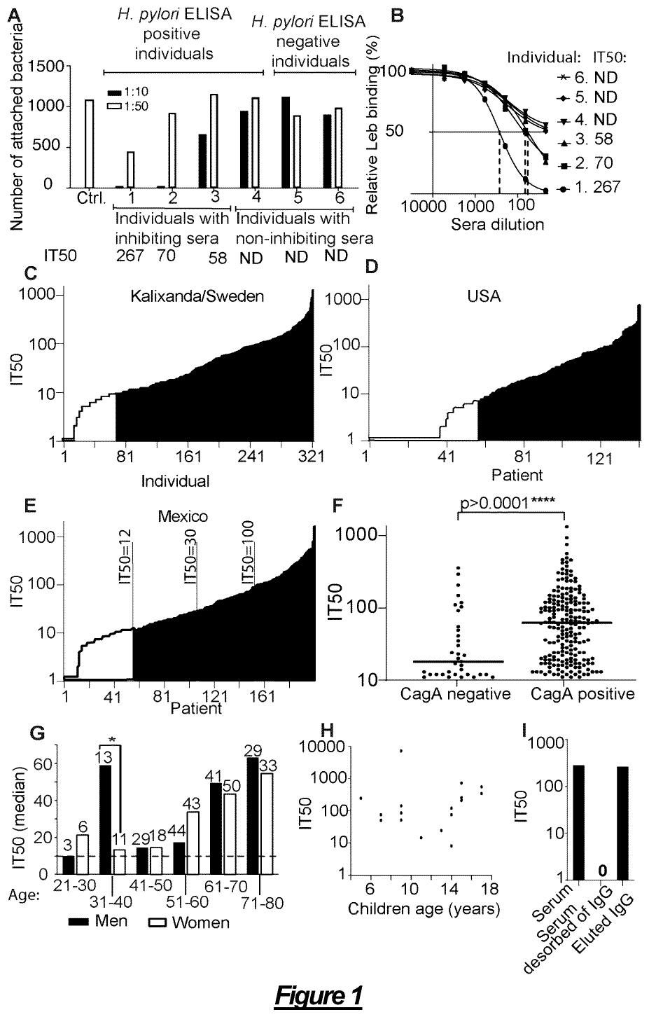

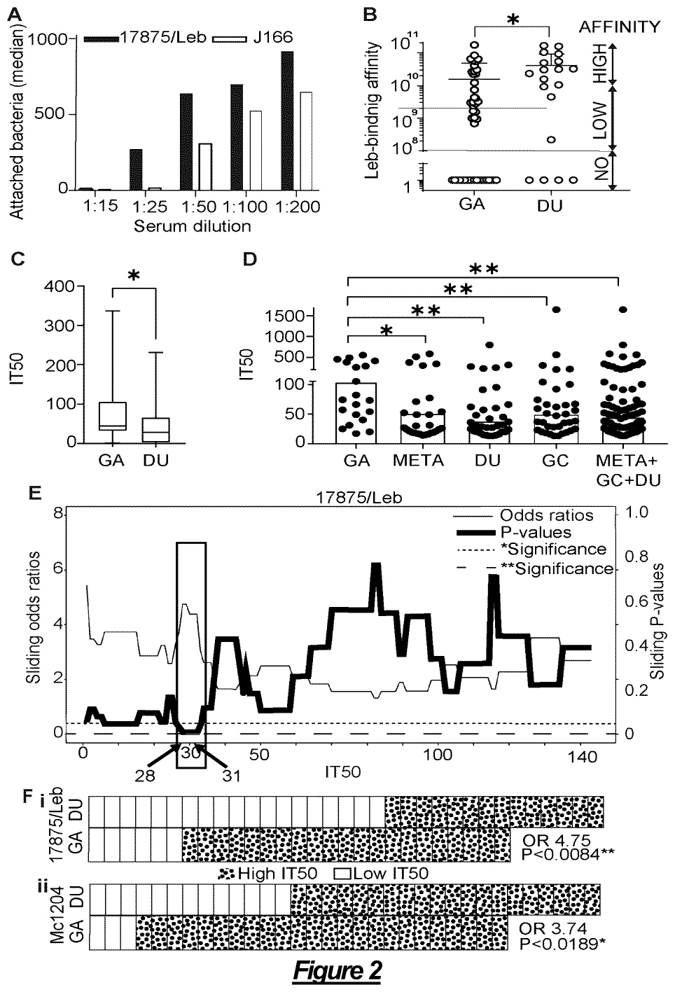

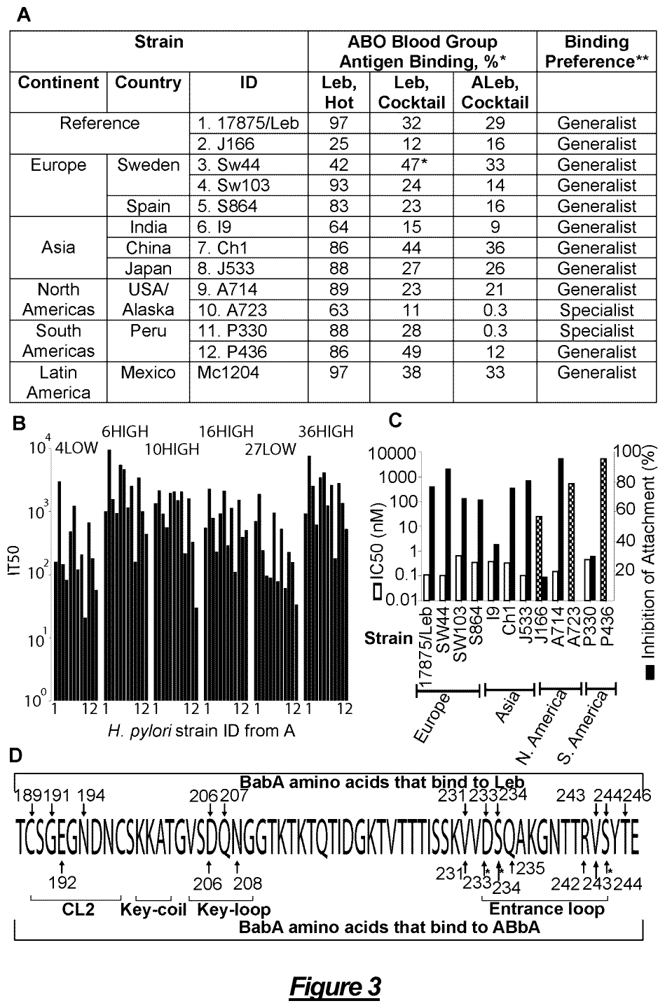

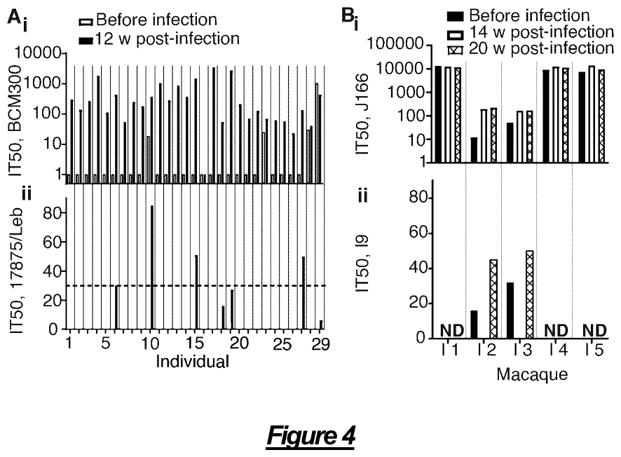

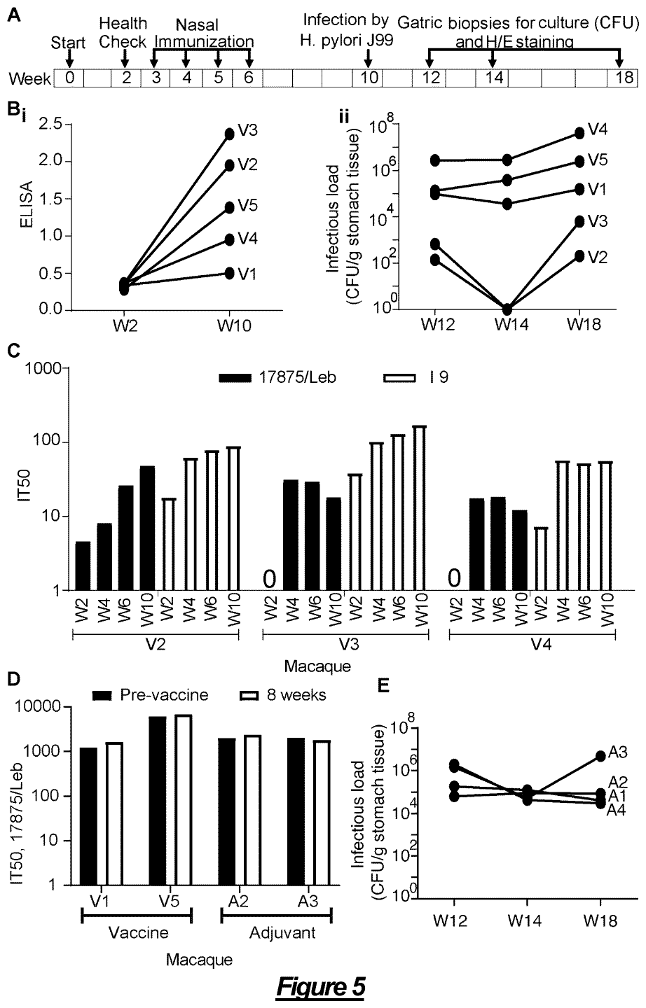

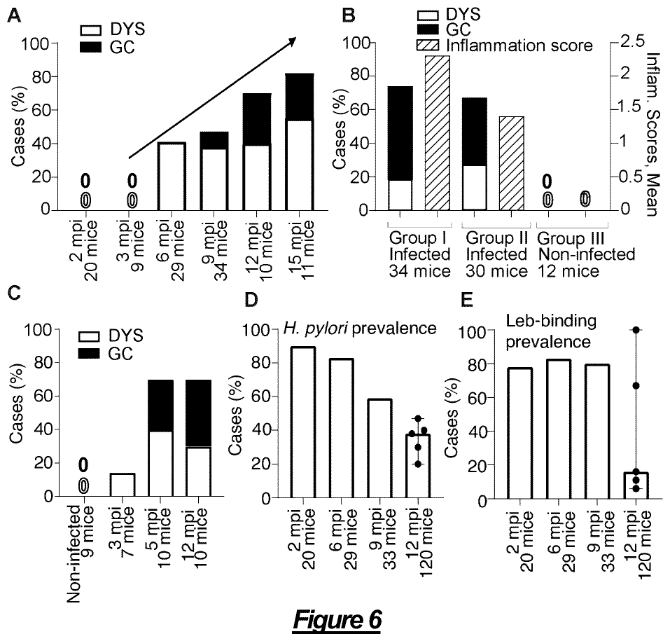

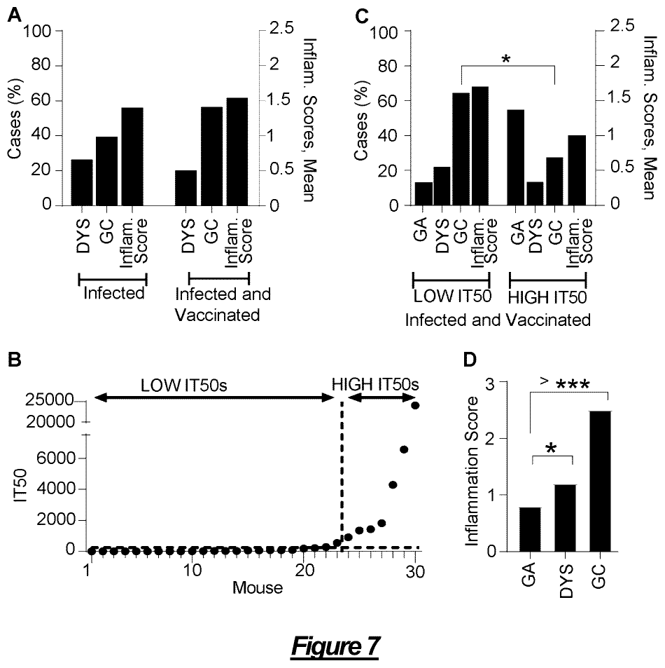

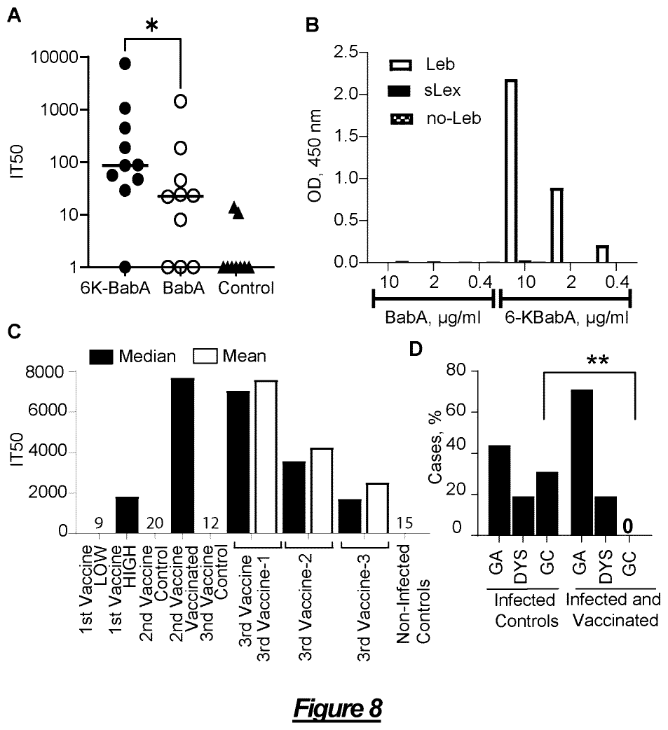

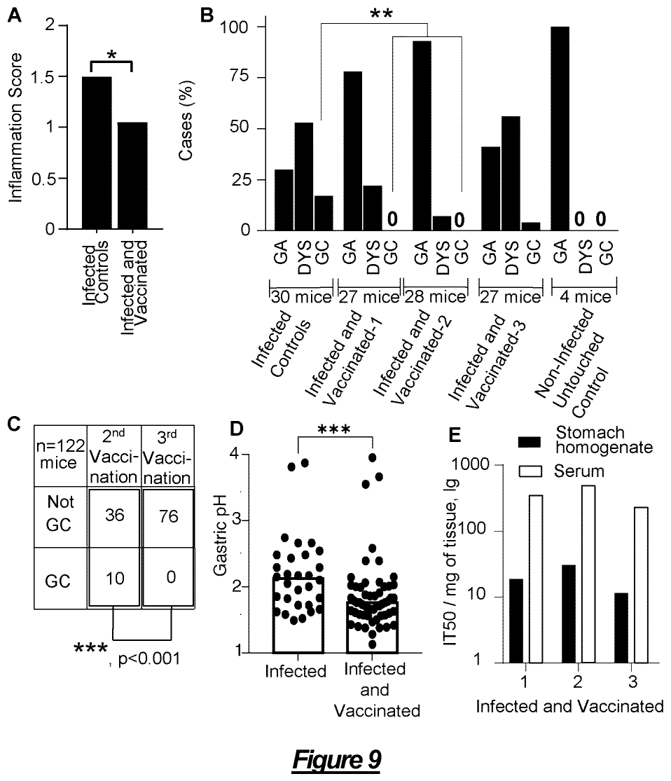

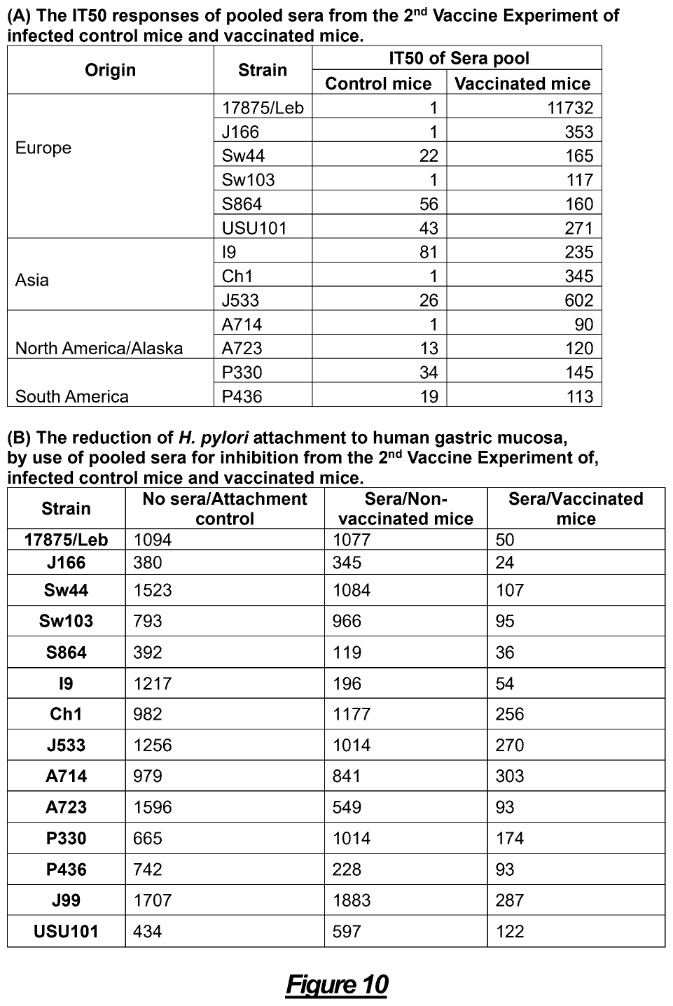

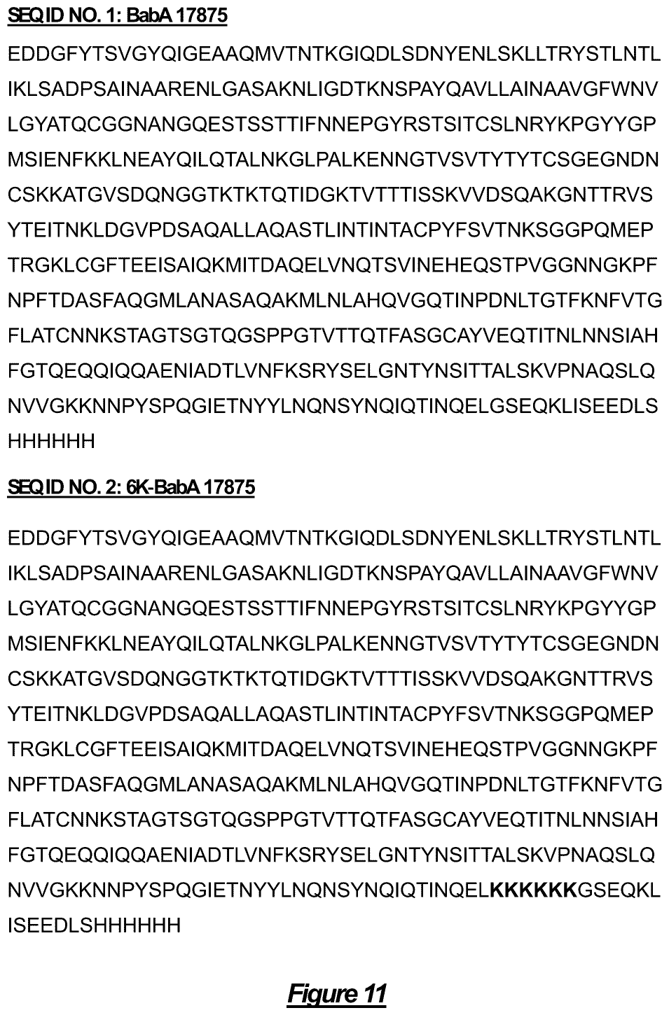

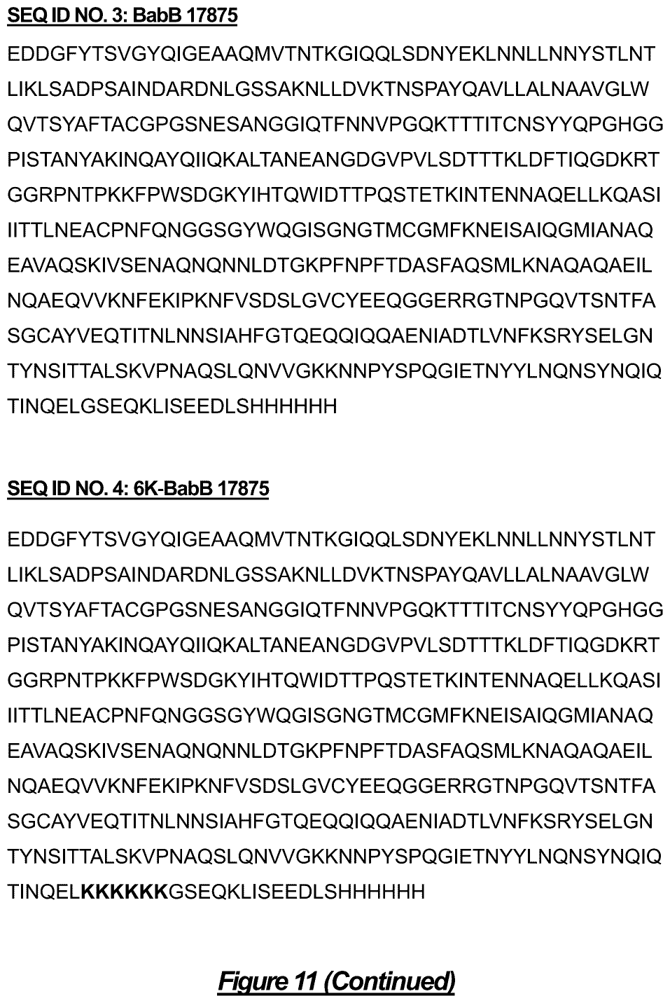

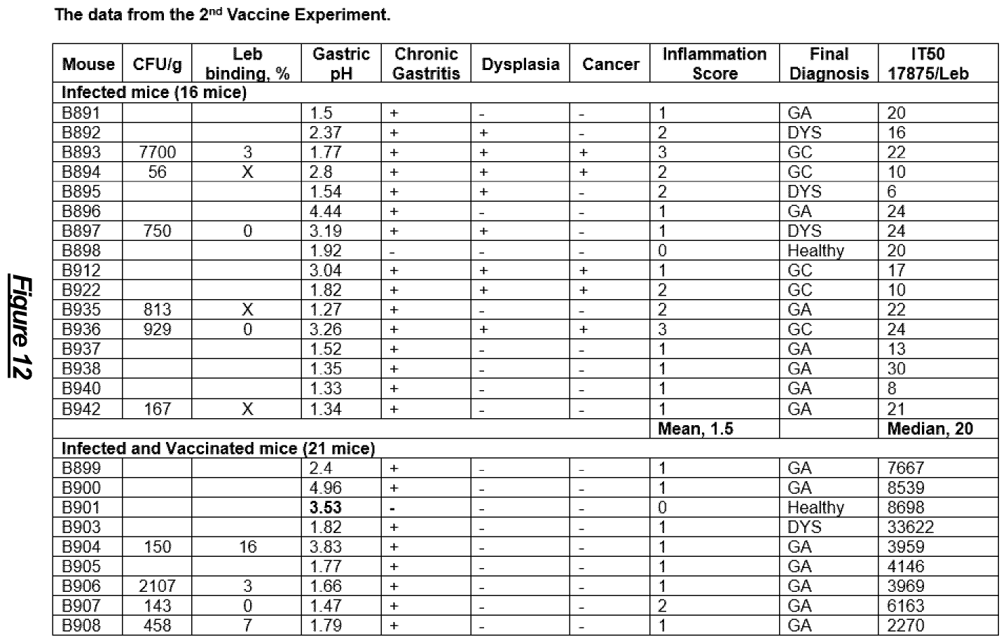

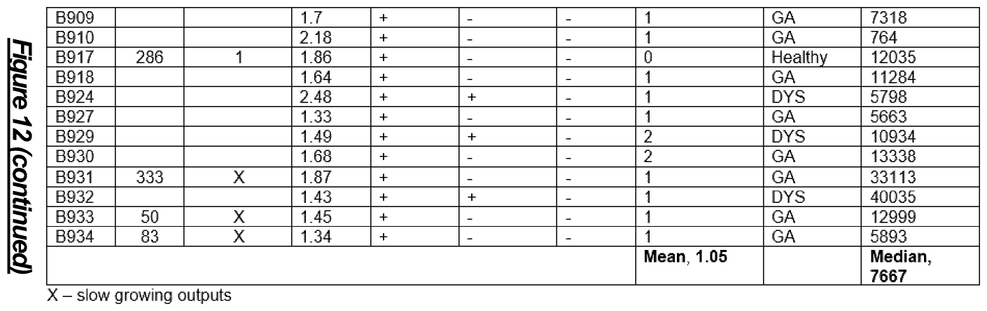

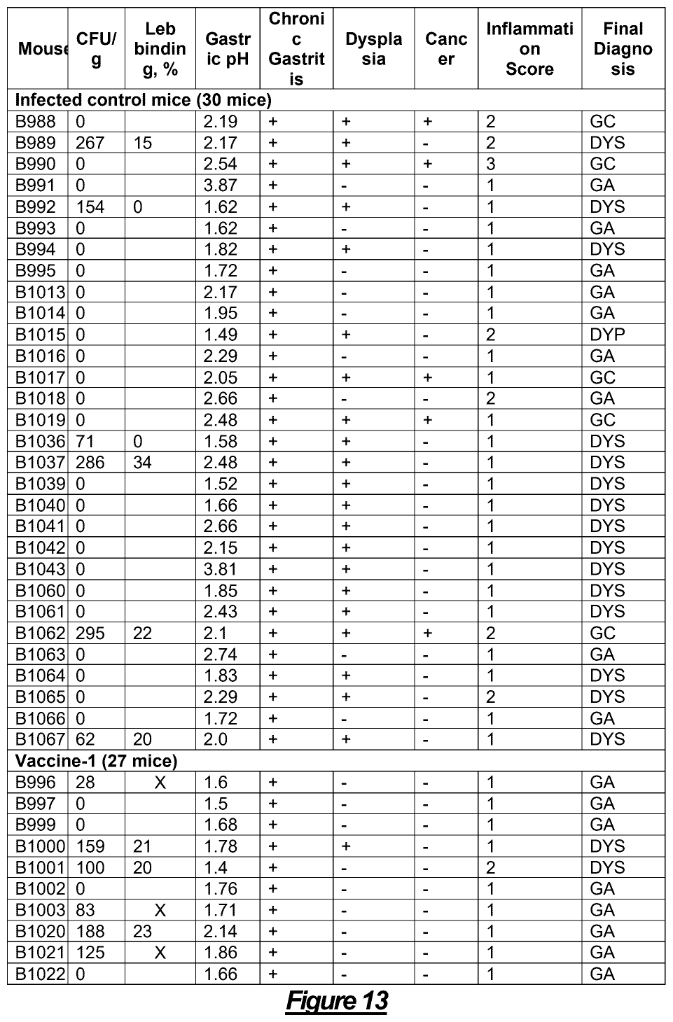

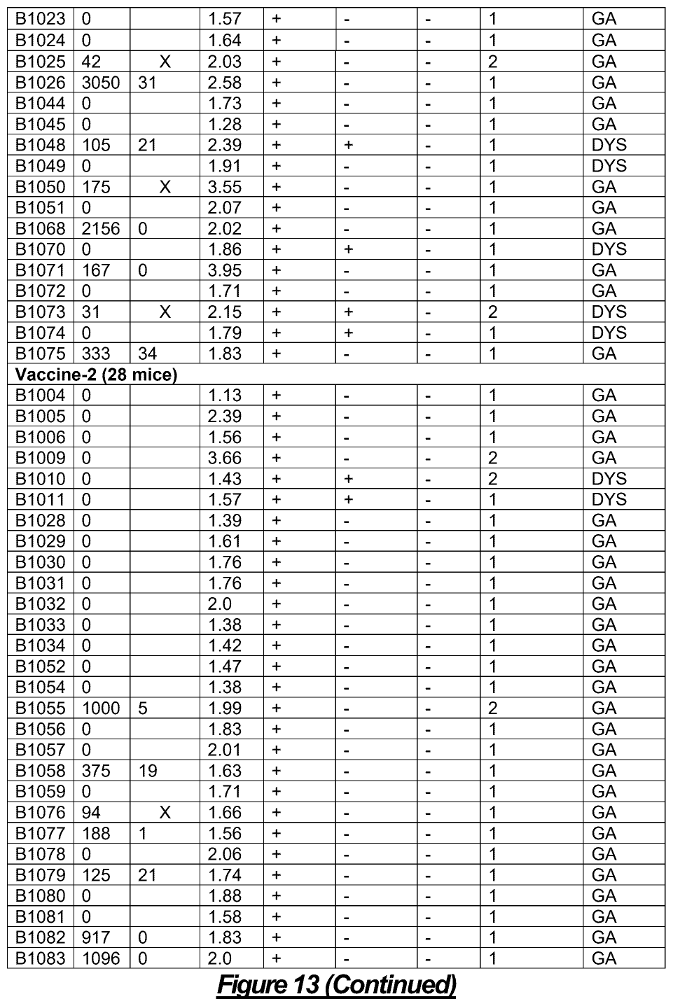

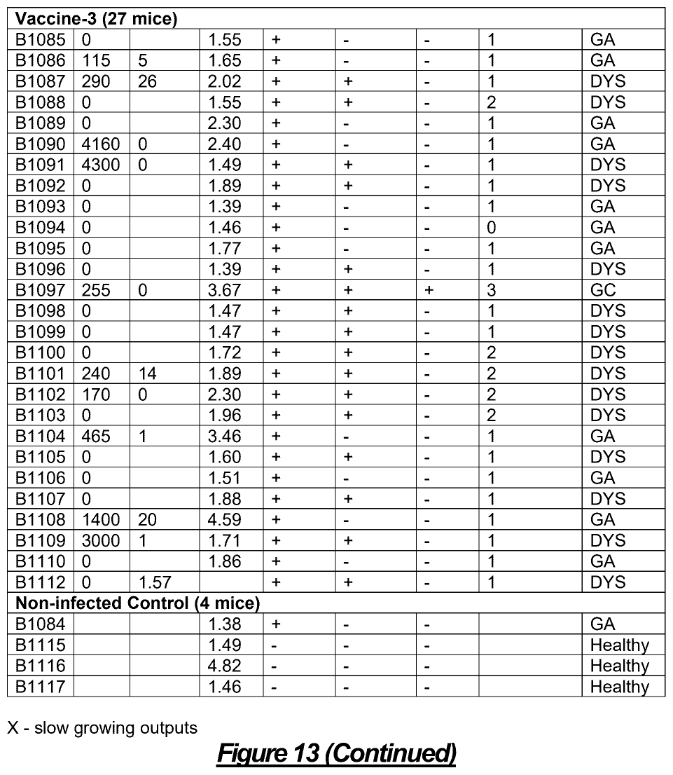

Panel (A) The serum sample from individual 1 in FIG. 1, Panel A and 1, Panel B, blocked attachment of H. pylori to human gastric mucosa. In the 1:25 dilution, the 17875/Leb (blue bars) and J166 (orange bars) strains demonstrated ˜10-fold difference in blocking (p<0.0001), with 28% and 3% residual attachment, respectively.

Panel (B) Among Swedish Leb-binding DU and GA isolates, ˜80% (12/15) of DU isolates demonstrated higher Leb-binding affinity compared to ˜50% (13/27) of the GA isolates (Wilcoxon p≤0.05*)

Panel (C) The IT50s are generally higher among the non-athorpic, i.e. healthier, gastritis GA-individuals compared to DU-subjects, median=44 vs. 28, p<0.0338* (Mann-Whitney test). Whereas the general H. pylori ELISA titers were similar for the two groups. The lower IT50 among the DU subjects does not relate to lower age, since the DU group of subjects is 10 years older.

Panel (D) The IT50s of the Mexico subject group (FIG. 1, Panel E); the 141 positive sera with IT50>13 were compared: The IT50s among the 25 GA-subjects (median=105) are higher compared to; (1) the 36 subjects with metaplasia (META), median=49, p<0.03*; (2) the 42 subjects with DU, median=39.5, p<0.007**; (3) the 38 subjects with gastric cancer (GC), median=48.5, p<0.008** and (4), the combined 116 subjects with META, DU and GC, median=46, p<0.0032** (Mann-Whitney test).

Panel (E) The sliding window X-axis shows the IT50s of the serum samples from GA vs. DU group of subjects (Panel (C)) tested by strain 17875/Leb. All the IT50s were used to calculate the full series of ORs (thin black line) and their corresponding p-values (thick black line) (IT50s from Panel (C)). The sliding window identified the critical interval IT50=28-31, which combines the highest OR 4.75, 95% CI: 1.58-15.86, with its highest significance p<0.0084**, indicated by the BOX (the significance level in dotted or hatched lines). Thus, the IT50<30 for 17875/Leb, was identified as Risk factor (Rf) for DU.

Panel (F) Individual sera from the 79 serum samples group of NAG vs. DU subjects tested (Panel (E)), where the critical IT50<30 for 17875/Leb was identified as an Rf for DU. The IT50<30 in the white boxes “Low” and IT50>30 in the spotted boxes “High”.

FIG. 3. Broadly blocking serum Abs and the human ABbA-mAb

Panel (A) The series of H. pylori strains that were tested for inhibition by human sera (Panel B) and by ABbA (Panel C); their binding strength properties and ABO-antigen binding preferences). The indigenous South American specialist isolates bind Leb 2.5-fold to ˜100-fold better compared to ALeb. In comparison, the common Generalist binding preference is defined as the Leb/ALeb-ratio interval from 1:1 to 1:2.5.

Panel (B) Serum samples from 6 Swedish individuals (from the KI, Sweden, FIG. 1, Panel (C)), were tested against 12 world-wide strains, including 10 ABO antigen-binding Generalist (G) and 2 Indigenous American Specialists(S). The results showing the relatively higher variation in IT50 titers for the two lower-inhibitory sera (individuals 4 and 27) compared to the four sera with higher inhibitory titers (individuals 6, 10, 16, and 36). The results suggest that the LOW sera displayed a higher level of discrimination for different strains compared to the HIGH sera that efficiently blocked the Leb binding of the majority of H. pylori strains.

Panel (C) shows the results of inhibition tests (IC50) by the cloned human broadly blocking monoclonal antibody ABbA (denoted ABbA), for the 12 world-wide H. pylori strains from Panel (A). ABbA blocked the Leb binding of 9 strains but less so for strain J166. The right Y-axis shows the results of a test of the blocking of H. pylori attachment by ABbA to human gastric mucosa in vitro by the series of H. pylori strains. The reduction in attachment by ABbA i.e., inhibition % closely reflected the ABbA IC50 for the corresponding strains, where the J166 strain with the higher IC50 was similarly modestly blocked by ABbA in terms of attachment (16%).

Panel (D) T The ABbA was co-crystallized with BabA and the interactions demonstrate three sites of Leb glycan mimicry; GM1; where ABbA VH (lilac) replaces the α1-2 “Secretor” fucose in the CL2 pocket; GM2, where ABbA VH replaces the α1-4 “Lewis” fucose; and GM3, where ABbA VH binds to the DSS-Triad residues. The panels show the 17875/Leb BabA carbo-hydrate binding domain where the amino acids that clusters in binding to both Leb and/or to ABbA are indicated.

FIG. 4. Induction of IT50 responses by challenge infection in humans and rhesus macaques

Panel (Ai): Among the 29 serum samples, 26 individuals responded with IT50s with the challenge strain H. pylori BCM300 (of African phylogeny. Four individuals (no: s. 10, 23, 28 and 29) had pre-challenge titers, and one individual showed no IT50 (Panel Aii). The phylogenetically distant strain 17875/Le identified five serum samples with bbAbs reached the protective level IT50˜30 (black bars).

Panel (Bi): Five SPF rhesus macaques were challenge infected with H. pylori J166. Two out of the five SPF animals, no. 2 and no. 3, demonstrated induction of an IT50 response, whereas the other three animals demonstrated high pre-challenge IT50 titers as tested with J166 (Bi).

Panel (Bii): Testing of the serum samples from the two animals no. 2 and no. 3 with the phylogenetically distant (unrelated) Indian strain 19 showed that they also induced IT50 responses of bbAbs. ND—Not Determined

FIG. 5. Vaccination and IT50 responses in rhesus macaques

Panel (A) shows the vaccination schedule with experimental design over 18 weeks (W).

Panel (Bi) ELISA detection of serum anti-BabA antibodies from vaccinated animals, whereas the adjuvant-only control rhesus macaques showed no induced ELISA signal. Panel (Bii) Colony forming units (CFUs)/g of stomach-pinch biopsy tissue, i.e., the infectious loads, were tested at 2 (W12), 4 (W14), and 8 (W18) weeks after the infection from vaccinated animals, compared to adjuvant-only control animals.

Panel (C) Test of serum IT50 responses in the vaccinated animals over 2-8 weeks (W) post-vaccination with strain 17875 (Europe) and strain 19 (India). The three vaccinated animals no. V2, V3, and V4 responded with de novo IT50s when tested with strain 17875, whereas the adjuvant-only control animals exhibit no IT50 responses. The 19 Indian strain demonstrated that the three vaccinated animals no. V2, V3, and V4 had responded with IT50=˜ 100 i.e., the bbAb activity.

Panel (D) illustrates that the vaccinated animals V1 and V5 and the adjuvant-only control animals A2 and A3 demonstrated high pre-challenge IT50 titers when tested with strain 17875. The A2 and A3 animal with high pre-challenge IT50 titers were not protected against challenge infection (Panel E).

In agreement, the vaccinated V1 and V5 animal with high pre-challenge IT50 titers were similarly not protected against challenge infection (Panel Bii).

Panel (E) The adjuvant-only control animals demonstrated stable high infection loads during the 8 weeks (W) of infection.

FIG. 6. The Leb-mouse gastric cancer animal model

The histopathology of the gastric cancer Leb-mouse model was evaluated by H&E-stained sections and blind scoring at the 12-month endpoint according to established criteria (Fox, J. G., and Wang, T. C. (2007). The mucosal inflammatory infiltration scoring is as described in the context of the relevant example, in the experimental section of the present disclosure.

The non-infected mouse does not display metaplastic or dysplastic changes and usually with no inflammatory infiltration (scored 0). In contrast, most infected mouse display inflammatory infiltration. Many of infected mouse display the intestinal metaplasia and numerous goblet cells, deformed, and branched gastric glands, glands with mucus-filled cysts (green arrow, dysplastic glands thickened with layers of nuclei, i.e., cell piling, low-grade cellular atypia. Approx. 30-50% of infected mice also display gastric cancer, sometimes with growth and penetration through the submucosal layer, and cancerous tissue characterized by massive inflammatory cell infiltration.

In FIG. 6, Panel (A) shows the incidence of dysplasia (DYS) (white box) and gastric cancer (GC) (black box) in the Leb-mouse model at 2, 3, 6, 9, 12, and 15 months post-infection (mpi), i.e., of chronic H. pylori USU101 infection.

Panel (B). The Leb-mice were infected with the onco-strain USU101 in two subsequent 12-month periods (Group I and II). The left Y-axis shows the percentage of mice with dysplasia (DYS) (white box) or gastric cancer (GC) (black box). The right Y-axis shows the inflammatory infiltration score (hatched).

Panel (C). The gastric cancer incidence at 12 months in response to antibiotic treatment and eradication of H. pylori infection. At 12 months, none of the non-infected mice or 3 months antibiotic-treated mice developed cancer, and only 1 out of 7 developed dysplasia. In contrast, at 12 months, the 5 months antibiotic-treated mice developed similar levels of dysplasia and cancer as the mice that were not treated with antibiotics during the 12 months period.

Panel (D) The H. pylori infection was found to be stable in the mice during the first 6 months of chronic H. pylori infection. The 12-month median CFU prevalence of 38% refers to 5 different 12-month infection tests of non-vaccinated mice (a total of 120 mice). All but 2 out of 272 infected mice demonstrated gastritis at 12 months, which suggests that all mice had indeed established H. pylori infection.

Panel (E) The BabA-mediated Leb-binding of the H. pylori infection was found to be stable in the mice during the first 9 months of chronic H. pylori infection, whereas at 12 months, most of the H. pylori infections had lost their Leb-binding activity. The 12-month median Leb-binding prevalence of 14% refers to 5 different 12-month infection tests of non-vaccinated mice (a total of 120 mice).

FIG. 7. The first vaccination experiment induced protection against inflammation and cancer

Panel (A) Two groups, each with 30 Leb-mice, were infected with the onco-strain USU101. One group was therapeutically vaccinated one month later, and all mice were evaluated at the 12-month endpoint. The left Y-axis shows the percentage of mice with dysplasia or gastric cancer. The right Y-axis shows the inflammatory infiltration.

Panel (B) The 30 immunized mice presented in increasing order of IT50, which made a natural divider of the two groups at IT50=about 1000 i.e., LOW IT50 vs. HIGH IT50 mice. The horizontal hashed line indicates the mean background IT50=<10 derived from the Group Il USU101-infected but non-immunized animals.

Panel (C) shows the incidence of gastric disease in the mice with LOW IT50 vs. HIGH IT50. The left Y-axis shows the percentage of mice with gastritis or dysplasia or gastric cancer. The right Y-axis shows the inflammatory infiltration.

Panel (D) The inflammation infiltration score was only 0.8 for gastritis but increased to 1.2 for dysplasia (p=0.05*) and almost doubled to 2.5 for the mice with gastric cancer (p=0.00000000005).

FIG. 8. The 6K-BabA protein demonstrates higher IT50 responses by immunization, and also higher Leb binding strength

Both the 2^{nd} and 3^{rd} vaccination tests induced high IT50 responses, with protection against gastric malignancy and cancer.

Panel (A) The IT50 responses of mice immunized with BabA w/wo 6K were tested by sera with strain 17875/Leb. Immunization with 1 μg 6K-BabA demonstrated 70% increased median IT50. Furthermore, immunization with 5-fold lower doses of 6K-BabA demonstrated 4-fold significant (one-star) increase in median IT50. The Inventors new results demonstrate that the 6K-tagged BabA-antigen provides immune response with higher IT50s of blocking and protective antibodies (Panel A).

Panel (B) Test by a serial dilution of BabA-antigen and 6K-BabA-antigen binding to Leb-glyco-conjugate immobilized in coated ELISA plates, show that rec6K-BabA-antigen demonstrates log-fold higher binding strength for the Leb-glycan. The new results demonstrate that the 6K-BabA-antigen exhibits enhanced structural stability of its carbohydrate binding domain (CBD), compared to recBabA, with resulting substantially higher Leb binding affinity (Panel B).

Panel (C) The distribution of median IT50s for the 1st, 2nd and 3rd vaccine experiments. In the 1st vaccination experiment, the LOW IT50 group demonstrated a median IT50 16, in contrast to the median IT50 1813 in the HIGH IT50 group (FIG. 7, Panel B). In the 2nd vaccination experiment a group of 37 Leb-mice were infected with the onco-strain USU101, and 21 mice were therapeutically vaccinated one month later and evaluated at the 12-month endpoint. The 16 non-vaccinated control mice demonstrated IT50=20, in contrast to the several log-fold higher IT50s, median IT50 7318, in the 21 vaccinated mice. Also, in the 3rd vaccination experiment (FIG. 9, Panel B) the 30 non-vaccinated control mice demonstrated the low IT50 15, in contrast to the median IT50 7026 in the 27 vaccinated mice in the Vaccine-1 (high antigen dose) group and, the median IT50 3557 in the 28 vaccinated mice in the Vaccine-2 (low antigen dose) group. In the Vaccine-3 group of 27 vaccinated mice (low antigen and no adjuvant) the median was merely IT50 1696. Whereas the 4 non-infected mice demonstrated median IT50=15.

Panel (D) The 21 vaccinated mice in Exp 2, demonstrated no cancer in contrast to 33% of the non-vaccinated mice (5/16) (p<0.01**). Four out of the five mice with gastric cancer developed invasive cancer.

FIG. 9. Both the 2nd and 3rd vaccination tests induced protection against gastric inflammation and protection against gastric malignancy and cancer and, in addition, vaccination preserved the natural gastric acidity.

Panel (A) The mean scores for gastric mucosal inflammatory infiltration. The non-vaccinated mice had a higher mean inflammation score of 1.5 vs. 1.05 (Wilcoxon: p=0.03*). And the inflammation intensity correlates with the severity of disease.

Panel (B) In the 3rd vaccine experiment, the 55 vaccinated mice in Vaccinanted-1 and Vaccinated-2 groups were all protected against gastric cancer, in contrast to the 5 (17%) cases of cancer in the non-vaccinated group of 30 mice (p<0.0043**). The 27 vaccinated mice in the Vaccinated-3 group demonstrated 1 (4%) case of cancer and 15 (56%) cases of dysplasia, which is similar to the non-vaccinated control group. The vaccinated-1 and vaccinated-2 groups of mice exhibited log-fold higher IT50 responses compared to the 30 non-vaccinated mice when tested with 17875/Leb (FIG. 8, Panel C).

Panel (C) Analyses by use of a 2×2 contingency table, and inclusion of the 46 mice from the 2nd Vaccination and the 76 mice from the 3rd Vaccination demonstrates that the 6K-BabA-vaccine raised full immuno-protection against gastric cancer (p<0.0001***).

Panel (D) In the 3rd vaccination test, the non-vaccinated mice demonstrated elevated gastric pH, whereas the vaccinated groups preserved the gastric acidity over the 12-month period, p=0.00073***. Thus, the vaccination preserved the natural gastric acidity.

Panel (E) The test of three vaccinated and three control mice showed that the IT50/mg stomach tissue protein was merely ˜10-fold lower compared to the corresponding IT50/mg serum protein. The non-vaccinated mice stomach homogenates demonstrated no IT50.

FIG. 10. Vaccination induced bbAb immune responses that blocked Leb binding (IT50) and, in addition, reduced H. pylori attachment to gastric mucosa

Panel (A): The IT50 of pooled sera from the 2nd Vaccine Experiment of Infected Control mice and Vaccinated mice. The sera of the vaccinated group of mice demonstrated bbAbs against the global series of H. pylori strains, including the challenge onco-strain USU101 (IT50=271) with median IT50˜200, with the antigen source, the 17875 strain, excluded from the mean calculation.

Panel (B): The reduction of H. pylori attachment to human gastric mucosa by use of pooled sera for inhibition from the 2nd Vaccine Experiment of infected control mice and vaccinated mice. The 1:10 diluted sera of the vaccinated group of mice reduced attachment to the human gastric mucosa by 95% for H. pylori 17875/Leb and by 80% for the challenge strain USU101, and ˜80% for the series of global isolates, compared to the sera of the non-vaccinated controls

FIG. 11. Sequence information

SEQ ID NO. 1 shows the amino acid sequence of BabA and SEQ ID NO. 2 shows the amino acid sequence of recombinant hexa-lysine modified BabA (6K-BabA) with the location of the hexa-lysine peptide (KKKKKK) at the C-terminal domain indicated in bold font. Similarly, SEQ ID NO. 3 shows the amino acid sequence of BabB and SEQ ID NO. 4 shows the amino acid sequence of recombinant hexa-lysine modified BabB (6K-BabB) with the location of the hexa-lysine peptide (KKKKKK) at the C-terminal domain indicated in bold font

FIG. 12. The full set of data from the 2nd Vaccine Experiment

The table comprises data for two cohorts: Infected (16 mice) and Infected and vaccinated (21 mice). The table is related to FIGS. 8, Panel C, 8, Panel D, 9, Panel A, 9, Panel C, and 9, Panel E.

FIG. 13. The full set data from the 3rd Vaccine Experiment

The table comprises data for four cohorts: Infected (30 mice), Vaccinated w High antigen dose (27 mice) and, infected and Vaccinated with Low antigen dose (28 mice) and, infected and Vaccinated with Low antigen dose but without adjuvant (27 mice). The table is related to FIGS. 8, Panel C, 9, Panel B, 9, Panel C, and 9, Panel D.

### DETAILED DESCRIPTION

### Definitions

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

The term “about” refers to a variation of a numerical quantity, wherein the extent of variation is from 0 to +5% and up to +10% while the quality remains unchanged. For example a homology of about 80% in relation to two amino acid sequences means that said two sequences can have as little as 72% homology provided that the corresponding proteins are functionally equivalent.

The term “comprising” is used in the terms to indicate that e.g. a composition includes the features enumerated in the claim, but without excluding the presence of other features or components in that composition, as long as they do not render the composition unworkable.

The term “broadly blocking antibodies” is used to describe antibodies that target conserved structural epitopes in the Leb-binding domain in the otherwise highly polymorphic BabA adhesin.

The term “immunogenically effective” is used to describe property of an antigen to produce an immune response, here mainly the ability to raise antibodies against said antigen.

The term “associated” in the expression “a gastric disease associated with H. pylori infection” is used to indicate that there is a link or correlation between the H-pylori infection and the disease. This association can take several forms:
Causative Association: Strong evidence suggests that the factor directly contributes to the development or progression of the disease. For instance, H. pylori is known to be a causative agent in developing peptic ulcers and certain types of gastritis.
Statistical Association: Epidemiological data show that the disease occurs more frequently in the presence of the factor than in its absence, but this doesn't necessarily prove causation. It indicates a correlation that warrants further investigation to determine if there is a direct cause-effect relationship.
Contributory Association: The factor may exacerbate or influence the severity of the disease, but it is not solely responsible for its onset. For example, certain lifestyle choices may not cause a disease but can worsen its prognosis if the disease develops.

The term “mucosal adjuvant” is used to describe adjuvants useful for mucosal delivery, and includes immunostimulatory molecules as well as delivery vehicles suitable for mucosal administration. In this context, focus is on immunostimulatory molecules such as, but not limited to, E. coli heat-labile toxin, dmLT, cholera toxin, multiple mutated cholera toxin (mmCT), CTA1-DD etc. For more information about cholera toxin-derived adjuvants combining the D-domain dimer from Staphylococcus aureus, see Ågren et al., 1998; Lycke & Schön, 2001; Eriksson et al., 2004)

The term “vaccine” is used to describe any immunogenic composition, regardless of its constitution, but the term is intended to particularly relate to mucosal vaccines, i.e. vaccines that are given orally or deposited directly on mucosal surfaces and trigger an immune response against a pathogen when said pathogen contacts the mucosal membranes of the digestive tract, such as the mouth, stomach, and/or the intestines.

The terms “prevent” and “prevention” are to be understood in a broad sense as specific, population-based and individual-based interventions for primary and secondary prevention, aiming to minimize the burden of diseases and associated risk factors. Primary prevention refers to actions aimed at avoiding the manifestation of a disease, whereas secondary prevention deals with early detection when this improves the chances for positive health outcomes. This comprises activities such as evidence-based screening programs for early detection of diseases and preventive drug therapies of proven effectiveness when administered at an early stage of a disease. The term “prevention” is primarily to be understood as the reduction of the incidence of a disease in a population infected with H. pylori or susceptible to being infected with H. pylori, compared to the incidence of the same disease in a control population not receiving the claimed composition or method of treatment.

Consequently, a first aspect of the present disclosure relates to a pharmaceutical composition comprising an immunogenically effective amount of an antigen for use in a method of preventing or treating gastric disease in a subject diagnosed with H. pylori infection, wherein said antigen is capable of inducing an antibody response sufficient to reduce mucosal attachment of H. pylori without eradicating the H. pylori infection; said antibody response specifically targets a conserved structural epitope in the Carbohydrate Binding Domain (CBD) of the ABO/Leb-Blood group Antigen-Binding Adhesin (BabA) of H. pylori; and said gastric disease is chosen from gastroesophageal reflux disease (GERD), chronic active gastritis (GA), peptic ulcer disease (DU), gastric ulcer disease, and gastric cancer.

According to an embodiment of said first aspect, the disease is gastric cancer, and wherein preventing gastric cancer is interpreted as the reduction of the incidence of gastric cancer in a population infected with H. pylori or susceptible to being infected by H. pylori, compared to the incidence in a control population not receiving said composition.

According to another embodiment of said first aspect and any embodiment thereof, the antigen is a recombinant modified BabA with an amino acid sequence having at least 80% identity, such as at least 85% identity, such as at least 90% identity, or at least 95% identity with the sequence defined by SEQ ID NO. 2 with the proviso that a continuous sequence of at least four positive amino acids is included in said sequence, preferably at the C-terminal of said sequence.

According to a preferred embodiment, said continuous sequence of at least four positive amino acid molecules is a sequence of at least four lysine molecules.

According to an alternative embodiment, said continuous sequence of at least four positive amino acid molecules is a sequence of at least four arginine molecules.

According to another, more preferred embodiment, said continuous sequence of at least four positive amino acid molecules is a sequence of at least six lysine molecules.

While the present results indicate that a hexa-lysine (6K) tag is preferred, the present inventors postulate that a multi-lysine tag of shorter or longer length can be used, for example two (2) to ten (10) lysine, for example two, four, six, eight or ten lysine molecules. As an alternative to lysine, it is contemplated that multiple positive amino acids, such as arginine modifications, e.g. 2-10 consecutive arginine molecules also contribute to increased stability and an improved effect.

According to an embodiment of the first aspect, freely combinable with any of the above embodiments, the composition further comprises another antigen chosen from variants of BabA having four disulfide bonds whereof the second disulfide bond spans seven amino acids between two adjacent cystein molecules.

According to an embodiment of the first aspect, freely combinable with any of the above embodiments, the composition further comprises a paralogue BabB or a variant thereof having at least 80% identity with the sequence defined by SEQ ID NO. 3 or SEQ ID NO. 4, with the proviso that, in variants of SEQ ID NO. 4, a continuous sequence of at least four positive amino acids is included in said sequences, preferably at the C-terminal of said sequence.

The above is supported inter alia by the results shown in FIG. 3C: It was seen that an antibody (ABbA) isolated from a subject in Stockholm/Europe does not block BabA/Leb binding to the same extent in subjects from an Asian population (China, Japan) and that it performs significantly worse in the indigenous populations in North and South America. The conclusion from this is that a future therapeutic composition, e.g. a vaccine would be more effective if the recBabA antigen is used in combination with a mutant (naturally adapted) sequence typical for China, Japan, and/or North/South America. A vaccine would then preferably comprise recBabA, plus 1 to 4 different geographical variants of BabA, with at least 80% homology with SEQ ID NO 1. Optionally said vaccine also comprises a BabB protein.

According to a preferred embodiment of said first aspect, and freely combinable with any of the above, the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

Unlike prior art approaches, targeting specific virulence factors of H. pylori, and aiming at preventing or eradicating H. pylori infection, the presently disclosed approach shifts the focus towards interfering with the disease-causing mechanisms while potentially preserving the microbial diversity of the stomach. The present invention instead aims at inducing an antibody response sufficient to prevent adherence of H. pylori to gastric mucosal cells without eradicating the H. pylori infection itself.

This method could lead to more efficient and less invasive treatments for H. pylori infections, with the broader implications of reducing the prevalence of gastric ulcers and cancer associated with the bacterium. Compared to traditional, prior art approaches, preserving the gastric and consequently the intestinal microbiota, may have many advantages. The present invention offers a significant improvement over traditional treatments, for example over the so called “triple therapy” which involves the administration of a proton pump inhibitor (PPI) such as omeprazole, esomeprazole, pantoprazole or other drugs used to reduce the acid in the stomach. This helps protect the stomach lining and allows the antibiotics to work more effectively. In the “triple therapy” a PPI is used in combination with two antibiotics (commonly clarithromycin and amoxicillin or metronidazole) taken for 7-14 days. In cases where resistance to antibiotics is a concern, or if initial treatment fails, quadruple therapy may be used. This typically includes a PPI, bismuth (which has an anti-bacterial effect), metronidazole, and tetracycline. Compared to such treatments, the present invention offers an alternative which does not involved expensive drugs, and which can help minimizing the use of antibiotics and the risk of antibiotic resistance. It is also contemplated that preserving the gastric and intestinal microbial diversity may have benefits for the human immune system and possibly minimize the development of allergies and autoimmune diseases.

A composition according to the first aspect or any embodiment thereof is preferably formulated as a vaccine comprising a pharmaceutically acceptable carrier and an adjuvant chosen from mucosal or systemic adjuvants that preferentially activate the humoral immune responses with induction of protective antibodies.

According to an embodiment of the above, said adjuvant is an aluminium-based adjuvant chosen from aluminum hydroxide, aluminum phosphate, and aluminum hydroxyphosphate sulfate.

According to an alternative embodiment, said adjuvant is a cholera toxin-derived adjuvant, more preferably CTA1-DD.

The choice of CTA1-DD for the vaccine composition is based on its recognized pathway of Th2-activation, i.e., humoral immune (preferentially antibody) responses and through protective Th17 cell induction, and less so of the Th1-pathway, i.e., the cell mediated immune responses such as cytotoxic T-cells. The Th1 pathways can be activated by other adjuvants, such as saponins, liposomes and polyinosinic-polycytidylic-acid (poly(I: C). This is of particular relevance since the innovators have results to show that the gastric cancer protective effects of vaccination is due to immune-responses of broadly blocking antibodies.

A second aspect of the present disclosure relates to a method for preventing a gastric disease in a subject diagnosed with H. pylori infection, comprising administering to the subject an effective amount of a composition containing an antigen wherein the administration of said antigen results in the induction of an antibody response that targets a conserved structural epitope in the Carbohydrate Binding Domain (CBD) of the ABO/Leb-Blood group Antigen-Binding Adhesin (BabA), sufficient to reduce mucosal attachment of H. pylori without eradicating the H. pylori infection in said subject, and wherein said gastric disease is chosen from gastroesophageal reflux disease (GERD), chronic active gastritis (GA), peptic ulcer disease (DU), gastric ulcer disease, and gastric cancer.

According to an embodiment of said second aspect, the gastric disease is gastric cancer and wherein preventing gastric cancer is interpreted the reduction of the incidence of gastric cancer in a population infected with H. pylori or susceptible to being infected by H. pylori, compared to the incidence in a control population not receiving said composition.

According to another embodiment, freely combinable with the above, the antigen is a recombinant modified BabA with an amino acid sequence having at least 80% identity, such as at least 85% identity, such as at least 90% identity, or at least 95% identity with the sequence defined by SEQ ID NO. 2 with the proviso that a continuous sequence of at least four positive amino acids is included in said sequence, preferably at the C-terminal of said sequence.

According to a preferred embodiment, said continuous sequence of at least four positive amino acid molecules is a sequence of at least four lysine molecules.

According to another embodiment, said continuous sequence of at least four positive amino acid molecules is a sequence of at least four arginine molecules.

According to another, more preferred embodiment, said continuous sequence of at least four positive amino acid molecules is a sequence of at least six lysine molecules.

According to an embodiment of the first aspect, freely combinable with any of the above embodiments, the method further comprises administering another antigen chosen from variants of BabA having four disulfide bonds whereof the second disulfide bond spans seven amino acids between two adjacent cystein molecules.

According to a further embodiment, freely combinable with any of the above embodiments, the method further comprises administering a paralogue BabB or a variant thereof having at least 80% identity with the sequence defined by SEQ ID NO. 3 or SEQ ID NO. 4, with the proviso that, in variants of SEQ ID NO. 4, a continuous sequence of at least four positive amino acids is included in said sequences, preferably at the C-terminal of said sequence.

According to a preferred embodiment of said second aspect, and freely combinable with any of the above embodiments, the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

According to a preferred embodiment of said method, the composition is formulated as a vaccine comprising a pharmaceutically acceptable carrier and an adjuvant chosen from mucosal or systemic adjuvants that preferentially activate the humoral immune responses with induction of protective antibodies.

According to an embodiment of the second aspect, said adjuvant is an aluminium-based adjuvant chosen from aluminum hydroxide, aluminum phosphate, and aluminum hydroxyphosphate sulfate.

According to an alternative embodiment, said adjuvant is a cholera toxin-derived adjuvant, more preferably CTA1-DD.

A third aspect of the present disclosure relates to an in vitro method for assessing the risk of developing gastric disease in a subject infected with H. pylori, said method comprising the steps of
providing a sample taken from said subject;
mixing the sample with a labelled receptor conjugate, ABO/Leb (Leb) conjugate, forming a mixture of sample and conjugate;
incubating said mixture with suspension of a H. pylori bacterial isolate or recombinant BabA;
determining the presence and/or amount of Leb vs the broadly blocking antibodies (bbAbs from the sample) bound to the H. pylori bacterial isolate or recombinant BabA; and
determining the 50% inhibition titer (IT50) of Leb binding in said sample, ie the titer (IT50) of the sample that can reduce the ABO/Leb binding to the H. pylori bacterial isolate or recombinant BabA with 50%,
   wherein an IT50 value below 50 in the sample is taken as an indication of an increased risk of developing a gastric disease.

According to an embodiment of said third aspect, the sample is a sample chosen from plasma, serum, saliva, urine, or gastric fluid.

According to a preferred embodiment, an IT50 value below 30 in the serum sample is taken as an indication of an increased risk for developing a gastric disease.

The global prevalence of H. pylori infection is high, with significant variation across different regions and populations. It's estimated that approximately half of the world's population is infected with H. pylori, making it one of the most common bacterial infections worldwide. Infection can be asymptomatic, and not everyone develops a gastric disease, but among those who do, gastric cancer is a very serious one. From a healthcare and pharmaceutical development perspective, understanding the epidemiology of H. pylori is crucial for designing effective treatment strategies and healthcare policies, particularly in areas with high prevalence. The present disclosure offers a possibility to identify individuals at higher risk of developing sequelae, and in particular serious and potentially life-threatening diseases such a gastric cancer.

A fourth aspect of the present disclosure relates to an in vitro method for classifying a subject with an H. pylori infection that would benefit from treatment with a composition as defined in any one of the claims 1-12 or the method according to claims 13-24, said method comprising the steps of:
providing a sample taken from the subject;
mixing the sample with a labelled Lewis B (Leb) conjugate, ABO/Leb (Leb) conjugate, forming a mixture of sample and conjugate;
incubating said mixture with suspension of a H. pylori bacterial isolate or recombinant BabA;
determining the presence and/or amount of Leb vs the broadly blocking antibodies (bbAbs from the sample) bound to the H. pylori bacterial isolate or recombinant BabA; and
determining the 50% inhibition titer (IT50) of Leb binding in said sample, i.e. the titer (IT50) of the sample that can reduce the ABO/Leb binding to the H. pylori bacterial isolate or recombinant BabA with 50%;
   wherein an IT50 value above background in the sample, where background level is determined with human H. pylori ELISA negative serum samples, is taken as an indication that said subject would benefit of a treatment with a composition as defined in any one of the claims 1-12 or the method according to claims 13-24, or wherein the subject would benefit from treatment with antibiotics.

According to an embodiment of the above fourth aspect, the sample is a sample chosen from plasma, serum, saliva, urine, or gastric fluid.

A positive IT50 signal is defined as an inhibition titer where the individual serum antibodies can reduce the signal of the labelled Leb conjugate bound to the suspension of a H. pylori bacterial isolate. A positive signal is an inhibition titer above background level and is defined as an inhibition titer that is above the titer that is obtained by use of pooled serum samples from one or preferable several ELISA negative, i.e., H. pylori negative individuals. Such sera are used to define the back-ground level (i.e., IT50 negative sera) because individuals who do not carry H. pylori infection and have never carried H. pylori infection, demonstrate no immune-response with IT50 inhibition titers of blocking antibodies against Leb-binding.

When classifying a subject with an H. pylori infection that would benefit from treatment, it is more important that the method is sensitive, so here a low-affinity Leb-binding isolate is used to achieve high sensitivity in identifying such subjects. By use of a low-affinity Leb binding isolate, the serum antibodies will out-compete the Leb binding more efficiently and hence produce an IT50 signal also in individuals with low IT50 levels. Thus, the method for detection of H. pylori infection will be much more sensitive using the use of a low-affinity Leb binding isolate.

Again, being able to accurately classifying patients would allow designing affordable and yet effective treatment strategies and healthcare policies, particularly in areas with high prevalence. The vaccine approach disclosed herein offers a proactive initiative, while the diagnostic approach enables a more accurate, and targeted reactive approach, for example reserving the administration of antibiotics for the subjects at risk of developing cancer.

A fifth aspect of the present disclosure relates to a kit for performing the method according to anu one of the third or fourth aspect or any embodiments thereof, wherein said kit comprises the reagents for performing the determination of IT50 for example by an Enzyme-Linked Immunosorbent Assay (ELISA), including a microtiter plate having wells wherein said wells are coated with recombinant BabA (or H. pylori bacterial isolate). Other immunoassay methods can also be used, such as but not limited to particle assisted immune assay methods, such as turbidimetric or nephelometric assays.

Consequently, according to an embodiment of the above aspect, said kit comprises the reagents for performing a turbidimetric determination of IT50, including for example a suspension of nanobeads with recombinant BabA immobilized to said nanobeads.

Such kit can be a kit for implementing any one of the methods as an enzyme-linked immunosorbent assay (ELISA), a radioimmunoassay (RIA), a lateral flow immunoassay (LFIA) etc. A person skilled in the art is well aware of the basic steps and elements of each assay type and can modify the assay and/or kit for performing the methods disclosed herein. In an ELISA, the antigen, recombinant BabA protein, is bound to the surface of a solid support, such as ELISA wells or plates, sample is added to said wells, and the broadly blocking antibodies (bbAbs) in the sample attach to the antigen. A secondary labelled antibody conjugate, capable of binding to the bbAbs, can then be added in order to allow quantification of the bbAbs in the sample. Alternatively, a Leb-conjugate that is with colorimetric labelled or fluorochrome labelled (or isotope labelled) can be used in the ELISA. The individual/subject serum broadly blocking antibodies (bbAbs) will then compete with the Leb conjugate in binding and will reduce the Leb conjugate colour signal. Such reduction in signal can be detected (by person skilled in the art) and the reduction in signal hence tells that the Individual carry bbAb because of H. pylori infection.

In RIA, the analyte, here the bbAbs, compete with radio-labelled antigen, e.g., Lewis B (leb) conjugate for binding to antibodies immobilized to a solid support.

In a LFIA device for detecting the presence or quantity of broadly blocking antibodies (bbAbs) against recombinant BabA residing in a test sample, the LFIA device comprises a porous membrane in liquid communication with a conjugate pad and a wicking pad:
said conjugate pad located upstream from a detection zone, said conjugate pad having detection probes with detectable markers and specific binding members for the analyte, and;
said detection zone having an immobilized first capture reagent, said first capture reagent being configured to bind to at least a portion of said analyte and analyte-conjugate complexes to generate a detection signal having an intensity;
a control zone located downstream from said detection zone, wherein a second capture reagent is immobilized within said control zone, said second capture reagent being configured to bind to said conjugate or conjugate-analyte complexes;
   wherein a sample containing said analyte is deposited on said conjugate pad and said sample moves toward said control zone, and wherein said analyte is a broadly blocking antibody (bbAb) against a recombinant BabA protein having an amino acid sequence with at least 80% homology with the sequence of SEQ ID NO. 1.

The ELISA technology is well-known and widely used in laboratories globally. However, when designed as a LFIA, the claimed kit offers a cheap and user-friendly assay, suitable for POC analysis, ambulatory medicin, and even home testing.

A sixth aspect of the present disclosure relates to an mRNA composition for use in preventing or treating a gastric disease in a subject diagnosed with H. pylori infection, wherein the mRNA encodes an antigen encapsulated in lipid nanoparticles, said antigen capable of inducing an antibody response sufficient to reduce mucosal attachment of H. pylori without eradicating the H. pylori infection; said antibody response specifically targeting a conserved structural epitope in the Carbohydrate Binding Domain (CBD) of the ABO/Leb-Blood group Antigen-Binding Adhesin (BabA) of H. pylori.

Associated herewith is a seventh aspect, which relates to a method for preventing a gastric disease in a subject diagnosed with H. pylori infection by eliciting an immune response in a mammal, comprising the steps of:
preparing a messenger RNA (mRNA) sequence encoding an antigen capable of inducing an antibody response sufficient to reduce mucosal attachment of H. pylori without eradicating the H. pylori infection; said antibody response specifically targeting a conserved structural epitope in the Carbohydrate Binding Domain (CBD) of the ABO/Leb-Blood group Antigen-Binding Adhesin (BabA) of H. pylori;
encapsulating said mRNA sequence in a lipid nanoparticle carrier to form an mRNA vaccine composition;
administering a therapeutically effective amount of said mRNA vaccine composition to the subject, wherein the lipid nanoparticle carrier facilitates delivery of the mRNA into the cells of the mammal;
expressing the antigen from the mRNA within the cells, thereby reducing mucosal attachment of H. pylori without eradicating the H. pylori infection in said subject.

The adaptability of mRNA technology makes it a powerful tool in developing vaccines against various diseases, particularly those where conventional vaccine approaches have failed or are less effective. The success of mRNA vaccines for COVID-19 has accelerated interest and investment in this field, speeding up the development process for new vaccine candidates. Based on the present disclosure, a person skilled in the art can set out to develop the claimed mRNA composition, as the present inventors have already identified an antigen that elicits an appropriate immune response. The optimization of the mRNA sequence for stability and efficient translation involves routine work. Appropriate software, reagents and laboratory methods and equipment is/are available to persons skilled in the art, for example Codon Optimization Tools. A skilled person can also choose to stabilize the mRNA against degradation using well-known methods in order to ascertain prolonged time of BabA protein expression in the human tissue. Ensuring efficient delivery into host cells and achieving sufficient expression levels of the antigen is important, but proven delivery systems such as lipid nanoparticles already exist.

According to an embodiment of any of the above aspects, the antigen is a BabA protein with an amino acid sequence having at least about 80% homology, such as at least 85% homology, such as at least 90% homology, or at least 95% homology with the sequence of SEQ ID NO. 1 or SEQ ID NO. 2. The considerable variation or relatively low degree of homology (at least about 80%) is motivated by the fact that BabA is an unusual protein which exhibits considerable amino acid variation due to mutations. When investigating samples from geographically diverse populations, it has been found that the sequences can differ about 20%, such as 19%, 18%, 17%, 16%, 15%, 14%, 13%, 12%, 11%, 10%, 9%, 8%, 7%, 6%, 5%, 4%, 3%, 2% or 1% and still exhibit strong binding and pathogenicity.

According to an embodiment, said BabA protein is isolated and purified from natural sources, but according to a preferred embodiment, said BabA protein is recombinant BabA.

According to an embodiment of said aspect, the composition comprises the paralogue BabB. Such paralogue may have at least 80% homology, such as at least 85% homology, such as at least 90% homology, or at least 95% homology with the sequence of SEQ ID NO. 3 or SEQ ID NO. 4, with the proviso that, in variants of SEQ ID NO. 4, a continuous sequence of at least four positive amino acids is included in said sequences, preferably at the C-terminal of said sequence.

The modified BabA and BabB proteins have several advantages. Adding a continuous sequence of positive amino acids not only improves the solubility and yield of the recombinantly expressed protein, it has also been shown to increase the structural stability of the protein. The inventors have shown that the BabA protein with the hexalysine peptide formed ordered crystals (PDB 8R4P) (https://www.rcsb.org/structure/unreleased/8R4P) in contrast to the BabA without hexalysine peptide suggesting that the structure was more stable, which allowed for the BabA protein to be organized into a crystal. Surprisingly, immunization experiments indicated a significantly improved immunogenicity of the hexa-lysine modified BabA protein, see e.g. Example 19. Here, the inventors tested the 6k-BabA antigen for improved structural stability and hence increased binding affinity for the cognate ligand, i.e., the ABO/Leb antigens, by application of a serial dilution of recBabA and rec6KBabA. The results demonstrated that rec6K-BabA binds the Leb antigen with several log-fold higher binding strength compared to BabA. The huge difference in binding affinity argues for that the 6KBabA has a greatly improved structural stabilization of its carbohydrate binding domain (CBD), compared to recBabA (FIG. 8, Panel B).

To sum, conventional methods have been focused on preventing infection or eradicating H. pylori infection, relying on massive use of antibiotics, and associated with constant setbacks in the form of re-infection. Instead, the present concept focuses on boosting the immune response of the body, allowing a subject to live with H. pylori infection but preventing or reducing the binding and minimizing the influence of the more aggressive strains of H. pylori. These conclusions are supported by the rhesus macaque vaccination tests, where four animals demonstrated high pre-vaccination IT50 titers. These four animals V1, V5, A3 and A3, were all readily challenge infected after vaccination, i.e., the four animals with high pre-vaccination IT50 titers were not at all protect against experimental infection. In addition, most of the ˜1000 individual sera tested from H. pylori ELISA positive individuals and subjects demonstrate IT50 titers of blocking antibodies. Nevertheless, all the ˜1000 individuals with IT50 titers carried H. pylori infection as verified by clinical/diagnostic ELISA tests. The vaccination results suggest that the IT50 titers of bbAbs do not protect against H. pylori infection, nor do the IT50 titers of bbAbs eradicate the ongoing H. pylori infections.

Thus, the new vaccine results represent a break-through in the light of the general concept that the human immune response is not capable of clearing H. pylori infections. The present inventors surprisingly show that mucosal immunization with the BabA vaccine delivers protection against severe gastric disease independently of eradication of the H. pylori infection. These results relate to the BabA-dependent adherent lifestyle of H. pylori, which provides the possibilities for therapeutic intervention by vaccine-induced blocking antibodies that competitively reduce H. pylori attachment.

The inventors suggest that the H. pylori pathogen can be turned into a gastric microbiome of more benign and commensal nature by suppression of its inflammatory adherence mode. An attenuated H. pylori microbiome might even be beneficial for the individual because childhood eradication of H. pylori is correlated with increases in asthma and allergies in addition to the reported increased risk for gastrointestinal reflux disease (GERD) and oesophageal cancer.

There are several advantages of a method of identifying subjects at risk of developing gastric disease and a kit for performing the method. The possibility to screen subjects, classify them according to severity of disease, risk and predicted outcome allows for better resource management in healthcare. It is not practically feasible to administer antibiotic treatment to all subjects infected with H. pylori, so identifying those with the highest risk of developing gastric disease and ultimately gastric cancer would give a powerful tool to physicians worldwide, and in particular in countries with scarce resources.

Further embodiments and the advantages thereof will be apparent to a skilled person from the following non-limiting examples.

### Examples

### METHODS (Experimental Model and Subject Details Relating to Examples 1-9)

H. pylori Strains

Laboratory strains: H. pylori 17875/Leb is an isolated single clone of H. pylori CCUG17875, and it binds to ABO/Leb antigens with high Leb-binding affinity, but not to sialylated antigens. The 17875babA1A2 strain is a null mutant with the two babA1 and babA2 genes deleted from H. pylori CCUG17875, referred to as the babA1A2-mutant. The H. pylori CCUG17874 binds sialylated antigens but not the ABO/Leb antigens (Aspholm, et al., 2006).

Clinical H. pylori isolates: The H. pylori isolates from Sweden (29 strains), Germany (4 strains), Spain (13 strains), Japan (13 strains), Alaska (11 strains), and Peru (38 strains) have been previously described (Aspholm, et al., 2004).

The H. pylori isolates Sw44, Sw103, S864, J533, A714, A723, P330, P436 have been previously described and the 13 Indian strains including isolate 19 and the J166 strain have been described (Bugaytsova et al., 2017). The 32 Mexican isolates from the UMAE Pediatria IMSS in Mexico City were isolated from subjects with GA (n=13) or DU (n=13). The H. pylori Ch1 strain was isolated from a Chinese individual with a family history of gastric cancer. H. pylori USU101 and a Rhesus macaque passage clone USU101AbabA with a natural babA deletion were as described by Bugaytsova et al., 2017.

H. pylori Culture Media

Cultures were performed with blood agar plates, and H. pylori cultures were grown in a mixed-gas incubator under micro-aerophilic conditions as described by Aspholm et al., 2006.

### Blood Group Antigen Conjugates

Two different types of fucosylated blood group antigen conjugates were used, namely 1) semi-synthetic Leb and ALeb-glycoconjugates (Leb-HSA and ALeb-HSA, respectively) with natural purified oligosaccharides covalently linked to human serum albumin (Isosep AB, Tullinge, Sweden). The conjugates were used for the radio-immuno assay (RIA) binding experiments.

### Serum Samples

Human subjects and sera: Procedures involving human subjects were approved by the relevant ethical boards. Informed consent was obtained from all human participants or legal guardians of participating minors.

### Monoclonal ABbA-IgG

Monoclonal ABbA-IgG was initially expressed and produced in Schneider cells and later by Absolute Antibody Ltd., Oxford Center for Innovation (New Road, Oxford, 0X1 1BY, United Kingdom) using transient expression in HEK293 cells.

In situ binding of H. pylori to human gastric mucosa histo-tissue sections (FIG. 1, Panel A) Bacteria were labelled with fluorescein isothiocyanate (FITC) (Sigma, St. Louis, MO), and in vitro bacterial adhesion was tested as described by Aspholm, et al., 2006. Slides were mounted with DAKO fluorescent mounting media (DAKO North America, Inc., CA, USA) and imaged with a Zeiss Axio Imager Z1 microscope (Carl Zeiss AB, Stockholm, Sweden) or the Leica Thunder microscopes. FITC-labelled H. pylori were digitalized from at least 3 identical fields of view from serial sections to quantify bacterial attachment. All images were evaluated by ImageJ. Briefly, micro-pictures were subjected to the threshold in order to separate objects of interest from the background and quantified by “Analyse particles”. Graphs and curves were created using GraphPad Prism 9 (San Diego, USA)

Inhibition of In Situ H. pylori Binding by Human Sera (FIG. 1, Panel A)

Inhibition tests were performed with the in situ binding methods as described above with the following modifications. FITC-labelled bacteria were first mixed with sera in SIA buffer at the dilutions shown in the figures on a slowly rocking table for 1 h at room temperature and then processed as described, Aspholm et al., 2006. The attachment of bacterial cells was digitalized and quantified as described above.

### Tests of Inhibition Titer (IT50)

### Labeling of Leb-HSA and ALeb-HSA by ¹²⁵I

The Leb-HSA and ALeb-HSA conjugates (IsoSep AB, Tullinge, Sweden) were I¹²⁵-labeled (I¹²⁵-Leb-conjugate) using the chloramine-T method (Aspholm et al., 2006).

### Analysis of BabA Binding Properties by RIA.

¹²⁵I-labeled Leb-HSA conjugate (“hot conjugate”) or ¹²⁵I-labeled ALeb-HSA diluted with unlabeled conjugate (“cocktail”) was mixed with 1 mL of bacterial suspension (OD₆₀₀=0.1) in blocking buffer. Following incubation, the bacteria were pelleted by centrifugation at 13,000×g, and the ¹²⁵I in the pellet and in the supernatant was measured using the 2470 Wizard2 Automatic Gamma counter (PerkinElmer, Waltham, MA, USA) giving a measure of binding activity (% binding).

### Analysis of Serum Inhibition Titers (IT₅₀) by RIA (FIG. 1, Panel B)

Serum samples from 960 H. pylori-infected subjects were tested for their ability to inhibit the binding of radiolabeled Leb-HSA conjugate (1125-Leb-conjugate) to selected H. pylori strains. To compare IT50s fairly between strains with varying maximum binding properties, all strains were calibrated in a pilot experiment to find the dilution corresponding to 10% 1251-Leb-conjugate binding. To ensure consistency of bacterial numbers and to aid pellet recovery, strains were diluted with H. pylori 17874, which does not bind Leb. For example, 17875/Leb at an OD600 nm=1.0 (2.5×10⁹ CFU/mL) was diluted 1:900 with H. pylori 17874 to reach 10% binding. Serial dilutions of the serum were made in blocking buffer, and 50 μL of ¹²⁵I-Leb-conjugate (0.01 ng/μL) was added to a final volume of 500 μL. After addition of 500 μl of the 17875/Leb and 17874 mixtures, the tubes were rotated for 17 h at room temperature. Samples were centrifuged (13,000×rpm for 13 min), and the ¹²⁵I-Leb-conjugates in the pellet and supernatant were measured to ascertain the bound and free conjugate amounts, respectively. The relative titer of the tested serum was defined as the dilution titer sufficient to reduce Leb binding to half the maximum value as determined by binding of the Leb conjugate in the absence of serum (IT50).

A micro-scale assay was established in a conical polystyrene 96-well plate (Thermo Scientific, Germany) that allowed the determination of the inhibitory titer with only 3 μl of serum. For each serum sample, a 1:3 serial dilution series (starting from 1:33) was established in 6 wells of the 96-well plate with a final volume of 60 μL. Then, 60 μL was added to each well of a mixture consisting of 1 ng of 1251-Leb conjugate and a suspension of H. pylori 17875/Leb (OD600=0.1) as well as H. pylori 17874 (OD600=0.1). All samples were diluted in blocking buffer. The 96-well plate was rotated for 17 h at room temperature and then centrifuged (4,000×rpm for 5 min), and the 1251-Leb-conjugate was measured separately in the pellet and supernatant to determine the IT50.

This micro-scale assay was additionally adapted to be performed like the original assay in 1.5 mL Eppendorf tubes. The procedure was identical to the 96-well assay with the difference that the centrifugation conditions post incubation were performed according to the original assay at 13,000×rpm for 13 min. IT50s were calculated identically as for the previous assay variations.

### Analysis of Odds Ratios (ORs) by Sliding Window Calculation (FIG. 2, Panel E)

The inventors started out by creating a database containing 4 entries derived from the milder symptoms of the infection (gastritis, G) and the more severe symptom of the infection (duodenal ulcer, DU). Both are further divided into High or Low depending on the selected cut-off value for the OR calculations.

The measurable parameter is the subject serum IT50 level, i.e. “How many times we can dilute the serum and inhibit 50% of the binding”. Depending on the test-strain, the IT50 for a given serum will change depending on strain's Leb-binding affinity (see IT50 method).

Because there is currently no definition of what a High or Low IT50 is in terms of antibodies towards the H. pylori binding adhesin, the inventors set out to determine “if there exists a numerical value of these IT50 for any given strain that indicates whether an individual subject is at risk for more severe disease progression?”

60 IT50 positive subjects (out of the original 79) were included, evenly/randomly divided between the DU and G diagnoses. These subjects had serum IT50s tested against multiple strains of different affinities for the Leb conjugate. For example, with the CCUG 17875/Leb strain (high affinity) IT50s were seen ranging from 1 (serums that inhibits but does not reach the 50% binding mark) and up to a few hundred. The mean and median IT50 was around 75. So, the inventors started testing the cut-off values at 2, and then slid up towards 150.

For every step an entry in the database was created. The first step was the temporarily defined “cut-off” (in this first entry it is equal to 2). The script then counted all observations of subject with the G diagnoses that had an IT50 below the current cut-off into the “Low G group”, everything with a G diagnose and above the cut-off was sorted into the “High G group”, and the same procedure was applied for the sera samples of DU diagnoses. For each tested cut-off the inventors were then left with 4 values that indicated how the subjects were distributed among the 4 groups, and a row-name was created corresponding to the cut-off it was calculated for.

The slider increased 1 more step and it iterated itself and created another entry for the cut-off equal to 3, and then continued to do so until the inventors ran across all of the different cut-offs we needed assessed by that particular test-strain. Because a sample could only move from the Low group to the High group, once a sample had moved into the High group it could never end up in another group again. So, after a certain time there were very few samples left that could “move” into another group. This means that the inventors always reached a level where there was no point in checking above a certain value as things would change with very low frequencies.

The final table was then comprised of rows containing the cut-off value and the subject distribution among the 4 groups. The OR was then calculated by dividing the ratio of “low DU/high DU” by the ratio of “low G/High G”. If the OR was equal to or close 1 this meant that there was no difference between the 2 different diagnoses and the subjects' IT50s. If the OR was greater than 1 this indicated that a high IT50 was connected to a milder diagnosis (G), and if the OR was less than 1 this indicated that a high IT50 was connected to a worse diagnosis (DU).

For each of the different cut-offs, the inventors ran a Fisher's exact t-test to test for significance, because low and high ORs did not necessarily correlate with significant observations. The ORs across the whole tested range for each strain was illustrated in a window containing all the ORs and how the OR changed as the cut-off level increased. On the second (right) Y-axis, the corresponding significance from the ORs was plotted. The classical 0.05 and 0.01 p-values were plotted as horizontal lines to visually show where (if anywhere) the ORs were presented as significant. The inventors suggest that these cut-offs can be used to indicate whether a subject should seek eradication (or future vaccine) treatment of their H. pylori infection or whether their own immune system will most likely be able to keep the infection in balance i.e., without disease progression.

### Purification of Human IgG (FIG. 1, Panel I).

A volume of 200 μl of human serum (total protein concentration 5 mg/ml) was diluted with 2.8 mL of binding buffer (20 mM sodium phosphate, pH 7.0). The sample was applied by a syringe onto a 1 mL HiTrap Protein G High Performance column (Cytiva, USA). The flow-through fraction with a total protein concentration of 4 mg/ml was saved for RIA analysis. The column was washed with 10 column volumes of binding buffer, and IgG was eluted as a 1 mL fraction with 10 mM glycine (pH 2.7) into tubes containing 100 μl 1M Tris (pH 9.0) and ≈2.5 mg/ml of protein. The protein concentration in the plasma and the flow through was determined by Pierce BCA Protein Assay Kit (Thermo Scientific, Rockford, IL, USA) according to the manufacturer's instructions. The purified IgG fraction concentrations were determined by absorbance at 280 nm with a Nanodrop ND-1000 Spectrophotometer (NanoDrop Technologies, In., USA). Human IgG was identified by immunoblotting with sheep anti-human HRP-conjugated antibody (GE Healthcare, USA) diluted 1:5000. The human IgG was detected as the 55 kDa heavy chain (HC) and the 30 kDa light chain (LC).

The BabA protein co-crystallized with ABbA-IgG (Fab) PDB Deposition ID.: D129212 (FIG. 3, Panel D)

To generate the BabA-ABbA complex, purified BabAAD (residues 25 to 460 of mature BabA) and ABbA-Fab were mixed at a 1:1 stoichiometric ratio. Crystal formation of the BabA-ABbA complex was enhanced by use of nanobody Nb-ER19. Nb-ER19 is a camelid VHH single domain antibody that binds the 3+4 alpha domain of BabAAD, stabilizes BabAAD, and facilitates its crystallization. Binding of Nb-ER19 is distant from the CBD and does not interfere with the binding of glycan receptors or of ABbA to BabA. BabAAD and Nb-ER19 were produced and purified. The ABbA-Fab was generated using the Pierce Fab Preparation Kit according to the manufacturer's instructions (ThermoScientific, cat #44985). The BabA-ABbA-Nb-ER19 complex (40 mg/ml in 20 mM Tris-HCl (pH 8.0) and 10 mM NaCl) was crystallized in 15% PEG6000, 0.1 M NaAc (pH 5.5), and 0.2 M CaCl2 by sitting drop vapor diffusion. For data collection, crystals were cryo-protected by brief transfer into the crystallization buffer supplemented with 15% glycerol, after which crystals were loop mounted and flash cooled in liquid nitrogen. X-ray diffraction data were collected from a single crystal at beamline ID29 of the ESRF (date 31/08/2014) at a wavelength of 1.072 Å to a final resolution of 2.7 Å. Diffraction data were integrated and scaled using XDS and XSCALE, and the structure was solved by molecular replacement using Phaser and BabAAD-Nb-ER19 (PDB: 5F7W) and an unrelated FAB (PDB: 1S5H) as search models. The final model was obtained after iterative cycles of manual building using Coot and maximum likelihood refinement against the X-ray data using Phenix Refine resulting in a model that contained two copies of the BabAAD-ABbA-Nb-ER19 complex per asymmetric unit, with an R and free R-factor of 22.9% and 28.3%, respectively, and 98.9% of the residues were in allowed regions of the Ramachandran plot. The BabA crystal structure was displayed with DNASTAR/Lasergene Protean 3D Software.

### Example 1. Human Serum Antibodies Block ABO/Leb Binding and Attachment of H. pylori Bacteria to the Gastric Mucosa

To test if human immune responses during chronic H. pylori infection raise factors that reduce bacterial adherence, the present inventors commissioned the test of sera from six individuals, four of whom were ELISA positive for H. pylori and two of whom were H. pylori negative. It was seen that three of the four positive sera samples fully inhibited or reduced epithelial attachment of the clinical isolate H. pylori J166 to the human gastric mucosa (FIG. 1, Panel A).

Two tests were used to identify the nature of the serum samples' inhibitory activity. First, the inhibitory activity was scored as the serum dilution at which half maximal H. pylori bacterial binding to Leb was lost, i.e., the 50% Inhibition Titer (IT50). Methods for determining IT50 in biological samples, such as serum, are well known to persons skilled in the art and can be performed e.g. by radioimmunoassay (RIA) or ELISA methods.

Here, serum samples from 960 H. pylori-infected subjects were tested for their ability to inhibit the binding of radiolabelled Leb-HSA conjugate (1251-Leb conjugate) to selected H. pylori strains.

In order to compare IT50s fairly between strains with varying maximum binding properties, all strains were calibrated in a pilot experiment to find the dilution corresponding to 10% ¹²⁵I-Leb-conjugate binding. To ensure consistency of bacterial numbers and to aid pellet recovery, strains were diluted with H. pylori 17874, which does not bind Leb. For example, 17875/Leb at an OD600 nm=1.0 (2.5×10⁹ CFU/mL) was diluted 1:900 with H. pylori 17874 to reach 10% binding. Serial dilutions of the serum were made in blocking buffer, and 50 μl of ¹²⁵I-Leb conjugate (0.01 ng/μL) was added to a final volume of 500 μL. After addition of 500 μL of the 17875/Leb and 17874 mixtures, the tubes were rotated for 17 h at room temperature. Samples were centrifuged (13,000×g for 13 min), and the ¹²⁵I-Leb-conjugates in the pellet and supernatant were measured to ascertain the bound and free conjugate amounts, respectively. The relative titer of the tested serum was defined as the dilution titer sufficient to reduce Leb binding to half the maximum value as determined by binding of the Leb conjugate in the absence of serum (IT50).

Hence, the IT50s tightly reflected the strength of the serum samples in inhibition of H. pylori attachment to the gastric mucosa (FIG. 1, Panel B).

Second, the IgG antibodies were specifically desorbed from the serum sample, which resulted in >95% loss of the inhibitory activity (FIG. 1, Panel I), suggesting that the broadly blocking activity resides in the IgG antibody fraction. These results show that individuals with H. pylori infection can raise attachment-blocking IgG antibodies that inhibit Leb binding and thus reduce H. pylori adherence to the gastric mucosa.

### Example 2. Worldwide High Prevalence of Serum IT50s

The results from FIG. 1, Panel A suggest that many individuals infected by H. pylori might be positive for Leb IT50s. Thus, in a pilot study, a group of 38 asymptomatic carriers of H. pylori infection from the Karolinska University Hospital (KI), Sweden, were tested, (Samples provided courtesy of Dr. Hammarstrom) and it was found that 22 (58%) of the sera reduced Leb binding with IT50s that ranged 100-fold, from ˜10 to >2000.

To determine if most individuals who are ELISA-positive for H. pylori raise antibodies that inhibit Leb binding, the inventors commissioned the testing of 742 individual serum samples from both healthy individuals and from subjects with gastric disease. For the IT50 tests, two H. pylori strains were used, i.e. 17875/Leb and J166, with high vs. low (FIG. 3, Panel A) Leb-binding affinity, respectively.

First, a group of 322 volunteers, all ELISA positive, i.e., H. pylori carriers, from the Kalixanda Study of the Swedish general adult population, was studied (Aro et al., 2006). This represented an essentially “gastric healthy” cohort. The inventors found that the Kalixanda serum samples similarly ranged 100-fold in IT50s and identified 270 (84%) IT50-positive sera whereas the remaining 16% did not demonstrate IT50s above background and were defined as negative (FIG. 1, Panel C). Notably, the IT50s demonstrated a strong correlation with the presence of antibodies against the onco-marker CagA (FIG. 1, Panel F), whereas the IT50-positive sera did not correlate with the H. pylori ELISA titers. Second, for comparison, 420 subjects with H. pylori infection and gastric disease from Ukraine, the US, and Mexico were tested, and 56%, 59%, and 72% were IT50-positive, respectively (FIGS. 1, Panel D and 1, Panel E). Leb binding tests showed that 81% (26/32) of the Mexican H. pylori clinical isolates bound to Leb, and the corresponding sera IT50s correlated with Leb binding (Wilcoxon p=0.036). Thus, the worldwide high prevalence of IT50s against Leb binding closely mirrors the 60%-80% world-wide prevalence of Leb binding among H. pylori isolates (Aspholm-Hurtig et al., 2004).

### Example 3. Serum IT50s Increase with Age

The IT50s in the Kalixanda population increased from mid-age to high-age in both women and men, with two exceptions—in the 31-40-year age group men had 4-fold higher IT50s (Wilcoxon rank, p=0.028) (FIG. 1, Panel G). It is noteworthy that the IT50 augmentations coincided with men's age of onset of duodenal ulcer (Aro et al., 2006) where the age matched increased IT50s might constitute protective antibody responses, as only 5% of the Kalixanda individuals had macroscopic gastric lesions (Aro et al., 2006). This suggests that the IT50s do not level out or decrease at higher age either on the population level or on the individual level.

To understand how early IT50s develop, the inventors tested children from 3 to 17 years of age (all ELISA-positive H. pylori carriers). It was shown that 47% (17/36) of the sera demonstrated IT50s from 5 years of age (FIG. 1, Panel H). The combined results showed that antibodies that inhibit Leb binding are formed already at an early age in response to H. pylori infections and are present and active throughout the lifespan.

### Example 4. The Serum IT50 Antibodies Bind to Structural BabA Epitopes

To characterize the epitope preferences for the antibodies that block Leb binding, the inventors tested serum samples from each of the Kl/Sweden, Ukraine, US, Mexico, and Kalixanda populations. The sera samples from all six global populations detected the Blood group Antigen Binding (BabA) protein, but only under semi-native conditions, suggesting that the antibodies do not recognize the denatured, linear epitopes but preferentially bind to structural epitopes in BabA.

Thus, testing of the 780 global individual H. pylori carriers suggested that (1) blocking antibodies (IT50s) are present in a vast majority of individuals, (2) IT50 levels are highly individual and do not correlate with the ELISA level, (3) the IT50 responses are more prominent against the oncogenic CagA-positive H. pylori infections, (4) the IT50s are active over the lifetime of the individual, and (5) the IT50s are raised against world-wide conserved structural BabA epitopes instead of antigenic variable linear epitopes.

### Example 5. The Serum IT50 is Derived from the Activity of Broadly Blocking Antibodies

To determine if the individual IT50 can also block Leb-binding of global, phylo-distant H. pylori strains, six Swedish serum samples were tested against 12 H. pylori isolates from Europe, Asia, and the Americas, including the IT50 reference strains 17875/Leb and J166. All six serum samples inhibited Leb binding of all 12 strains, i.e., all individual sera exhibited broadly blocking antibody activity. The IT50s against strain 17875/Leb reflected the general inhibition strength of the six serum samples. Notably, the two LOW inhibitory sera preferentially inhibited the H. pylori strains J166 and 19, which were lower in Leb binding affinity. Reciprocally, the four HIGH inhibitory sera were less discriminating in inhibiting the 12 strains (FIG. 3, Panel B). The results on the sera broadly blocking antibody activities help explain why the series of 780 global serum samples were so efficient in inhibiting strains 17875/Leb and J166 (FIGS. 1 and 2) that both originate from Europe.

### Example 6. H. pylori with Higher-Affinity Leb-Binding is a Risk Factor for Overt Gastric Disease

To understand how Leb binding affinity affects the serum inhibition of H. pylori attachment to the gastric mucosa, strains 17875/Leb and J166, with high vs. low Leb binding affinity, respectively, were exposed to serum from individual 1 from FIGS. 1, Panel A and 1, Panel B. A 25-fold dilution of the serum reduced the attachment of strain J166 10-fold compared to strain 17875/Leb (p<0.0001) (FIG. 2, Panel A). Thus, the low-affinity Leb-binding strain J166 was more efficiently inhibited by serum antibodies in terms of both Leb binding (FIG. 1, Panels C, D, E) and attachment to the gastric mucosa (FIG. 2, Panel A). Thus, the series of Swedish H. pylori isolates from subjects with gastritis (GA) or duodenal ulcer (DU) was re-analysed for their Leb affinity because the CagA, VacA, BabA “triple positive” virulence markers are present in almost all DU and gastric cancer strains (Gerhard, M. et al., 1999.). Indeed, the DU strains demonstrated higher binding affinity (Wilcoxon p≤0.05) (FIG. 2, Panel B). Similarly, the Mexican Leb-binding DU isolates demonstrated higher binding strength compared to the GA isolates (Wilcoxon signed-rank test, p=0.0015).

### Example 7. Low IT50 is a risk factor for overt gastric disease

Because more blocking antibodies, i.e., higher IT50s, are required to prevent the HIGH-affinity Leb-binding H. pylori strains from adhering to the gastric mucosa, and because the “triple-positive strains” with BabA of HIGH Leb-binding strength constitute a risk factor for overt gastric disease, it was tested if a low individual IT50 can be regarded a risk factor for overt gastric disease. The new results propose that only higher IT50s can block the strongly binding DU strains. This prompted us to test if individual sera with low IT50 i.e., low level of blocking antibodies, constitutes a risk factor (Rf) for severe gastric disease. First, we selected a series of 79 serum samples (60 with IT50 titers) from individuals with common gastritis (GA) and subjects with DU. The IT50s ranged several 100-fold, but the GA sera were significant higher in IT50s (FIG. 2, Panel C). Intrigued by the lower IT50s among the DU subjects, we tested also the group of 200 Mexican subjects with gastric disease (FIG. 2, Panel D): GA with metaplasia, DU or gastric cancer for comparison to the sera IT50s in subjects with the common gastritis (GA). Also, in this group of 200 subject, subjects with G presented significant higher IT50s compared to the subjects with DU. Notably, the GA subjects demonstrate significant higher IT50 also compared to the subjects with metaplasia and significant higher compared to the subjects with gastric cancer (FIG. 2, Panel D). The IT50s of the subject group (FIG. 2, Panel D); the 141 positive sera with IT50>13 were compared: The IT50s among the 25 GA-subjects (median=105) are higher compared to; (1) the 36 subjects with metaplasia (META), median=49, p<0.03*; (2) the 42 subjects with DU, median=39.5, p<0.007**; (3) the 38 subjects with gastric cancer (GC), median=48.5, p<0.008** and (4), the combined 116 subjects with META, DU and GC, median=46, p<0.0032** (Mann-Whitney test).

Second, the full range of odds ratios (ORs) and their p-values for sera samples with high vs. low IT50 for the NAG vs. DU group were calculated. By using sliding windows (FIGS. 2, Panel E and 2, Panel Fi) and ROC diagrams, an IT50 of approx. <30 was identified as a risk factor (Rf) for DU, by strain 17875/Leb, where the highest OR of 4.75 coincided with the highest significance (FIG. 2, Panel E).

The inventors also tested the high-affinity Leb-binding Mc1204 isolate from the cognate Mexican group and identified IT50 approx. 15 as an Rf where the highest OR of 3.74 coincided with the highest significance (FIG. 2, Panel Fii), i.e. the two strains produced similar low IT50s as Rfs. The critical ORs were confirmed by ROC diagrams and by permutation tests. This series of results suggest that individuals with low IT50 serum levels are at a higher risk for duodenal ulcer disease.

For comparison, the inventors next calculated the odds ratios (ORs) for the series of severe gastric disease in FIG. 2, Panel D; metaplasia, IT50=31, OR 4.97, p<0.014*; DU, IT50=48, OR 4.25, p<0.011*; gastric cancer, IT50=39 and 69, OR 4.25, p<0.019 vs. 0.01. Thus, the different types of gastric disease all display the similar low IT50s for the significant OR. The gastric cancer display two IT50s (39 and 69), which can reflect the many decades between the start of the Correa cascade vs. the cancer diagnosis. The series of results suggest that individuals that respond to their H. pylori infection with lower IT50s are at higher risk for severe gastric disease, such as metaplasia, DU or gastric cancer.

Notably, the 1:15 serum dilution required to completely block attachment to the gastric mucosa of the high affinity binding strain 17875 (FIG. 2, Panel A) corresponded to an IT50=10 when tested using 17875. Thus, the IT50 serum titers need to exceed the critical Rf IT50 approx. 30 in order to provide for the functional, necessary IT50=˜ 10 level of adherence blocking efficacy in the gastric mucosal lining, i.e., on distance from systemic circulation and on distance from local antibody producing immune cells. The present results suggest that DU subjects often carry the high-affinity Leb-binding H. pylori, i.e., the strains that require higher IT50 sera for blocking of Leb binding and hence reduction of H. pylori attachment; however, the DU group of subjects often presented with lower and thus insufficient IT50 titers of protective bbAbs, and similar to the individuals that respond to their H. pylori infection with lower IT50s are at higher risk for severe gastric disease, such as gastritis with metaplasia, or gastric cancer.

### Example 8. Cloning and Identification of the bbAbs

For a better understanding of the mAbs that correspond to the human sera IT50s, the inventors next cloned and expressed the natural blocking mAbs from their original source, i.e., from individuals with high IT50s.

Notably, ABbA mAb reduced both the Leb binding (tested by its inhibitory concentration 1.e. the IC50 defined as IT50 by concentration of the ABbA protein) and the attachment to human gastric mucosa of the majority (9/12) of world-wide H. pylori strains and hence exhibited a broadly blocking activity (FIG. 3, Panel C). Thus, it was concluded that ABbA can protect the mucosa from H. pylori attachment, although IC50 was higher, and reduction of attachment was lower among Asian strains and in particular strains from the indigenous Americas populations.

Thus, the antibody (ABbA) isolated from a subject in Stockholm/Europe does not block BabA/Leb binding to the same extent in subjects from an Asian population (China, Japan) and that it performs significantly worse in the indigenous populations in North and South America. The conclusion from this is that a future therapeutic composition, e.g. a vaccine would be more effective if the recBabA antigen is used in combination with a mutant (naturally adapted) sequence typical for China, Japan, and/or North/South America. A vaccine would then preferably comprise recBabA, plus 1 to 4 different geographical variants of BabA, with at least 80% homology with SEQ ID NO 1. Optionally said vaccine also comprises a BabB protein.

Third, to understand the epitope preferences that might explain the broadly blocking ABbA properties, the semi-native-immunoblot method was applied, and it was found that ABbA bound only to folded structural BabA epitopes and not to denatured linear epitopes. Thus, the ABbA mAb binds BabA similarly to the human (polyclonal) serum antibodies from infected individuals.

Thus, the ABbA mAb exhibited similar high-affinity binding and specificity for BabA as the polyclonal subject sera with a remarkably efficient broadly blocking activity for worldwide H. pylori strains.

### Example 9. ABbA Performs Glycan Mimicry in its Broadly Blocking Binding to Structural Epitopes

The crystal structure can be determined by methods known to persons skilled in the art.-Here, the purified BabA-AD (Adhesin Domain) (residues 25 to 460 of mature BabA) and ABbA-Fab were mixed at a 1:1 stoichiometric ratio. The ABbA-Fab was generated using the Pierce Fab Preparation Kit according to the manufacturer's instructions (ThermoScientific, cat #44985). X-ray diffraction data were collected from a single crystal at beamline ID29 of the ESRF (date 31/08/2014) at a wavelength of 1.072 Å to a final resolution of 2.7 Å. PDB Deposition ID.: 129212.

The BabA body domain is formed by a 4+3-helix bundle with a four-stranded B-plate insertion that forms the CBD located at the top of the body domain (Moonens et al, 2016). In the CBD, the CL2 cysteine loop and the Key-loop (DL1) and Entrance-loop (DL2) all contribute with binding sites for Leb. The principal determinant of ABO/Leb antigens, the α1.2 secretor fucose, is bound by a pocket formed by the conserved CL2 loop, whereas Leb's reducing end is bound by the DSS-Triad in the Entrance-loop 13,25 (FIG. 3, Panel D). In the co-crystal structural model, ABbA binding targets these same three areas where the variable heavy (VH) residue W102 perfectly inserts into the α1.2 secretor fucose loci whereas VH residue L31 resides in a pocket formed by V231 in a position equivalent to the α1.4 Lewis fucose. In addition, VH binds to both D206 and N208 in the Key-loop and to the DSS triad (FIG. 3, Panel D)).

Thus, ABbA competes with Leb in binding to BabA through functional glycan mimicry and binds the majority of amino acid residues in the CBD that are critical for Leb binding. Notably, ABbA W102 and L31 provide surrogates for the secretor and Lewis fucose by filling the two hydrophobic pockets, glycan mimicry 1 (GM1) and 2 (GM2), respectively, where the hydrophobic determinants of the L-enantiomeric fucose residues reside.

Thus, the triple-locus glycan mimicry performance of ABbA provides a universal fit to the CBD despite its polymorphic landscape, where Leb and ABbA bind to the same amino acid residues, as illustrated in FIG. 3, Panel D. This triple-locus glycan mimicry performance of ABbA works “hand-in-glove” to protect the ABbA mAb from immune escape through common antigenic variation performed by BabA and, helps explain the ABbA broadly blocking properties.

### Methods (Experimental Model and Subject Details Relating to Examples 10-20)

### Human Sera Collection

The Novartis challenge infection study: A total of 29 serum samples were obtained from healthy volunteers who participated in an immunization study with a parenteral vaccine against H. pylori and challenged with the H. pylori BCM-300 strain. The volunteers all tested negative for H. pylori infection by serological, faecal antigen, 13C urea breath tests, and gastrointestinal endoscopy with 16 biopsies from the stomach antrum and body for rapid urease testing, H. pylori culturing, and histopathological molecular and immunological analysis. Sera were collected before challenge infection and at 12 weeks after.

### Macaque Sera, Prophylactic Vaccination, and Challenge Infection

Challenge infection of the rhesus macaques and tests for sera IT50: Animals and the experimental design were essentially according to Hansen, L. M. et al., 2017. Colony-bred, SPF male and female rhesus macaques between the ages of 2 and 7 years that were free of H. pylori infection. Four experimental groups were challenged by gavage with 109 CFU/2 ml of H. pylori J166 WT. IT50 titers were determined before and at 14 and 20 weeks after infection.

Vaccination with BabA Antigens, Challenge Infection of the Rhesus Macaque Animals, and Tests for Sera IT50

Animals and experimental design: Two groups (N=5 and N=4) of SPF rhesus macaques were used for the 18-week experiment. The sample size was chosen to detect a 1.80 difference with 80% power, keeping in mind considerations of animal cost and availability. After identification of SPF macaques by endoscopy (week 0-2), the animals were orally immunized once per week for 4 weeks (week 2-6) with vaccine (CTA1-DD plus BabA) or adjuvant only (CTA1-DD). Four weeks after completion of immunization (week 10), all animals were challenged with rhesus-adapted H. pylori J99. Gastric biopsies obtained 2, 4, and 8 weeks after challenge (week 12, 14, and 18, respectively) were used for quantitative H. pylori cultures and for histopathology to assess the inflammatory response. Serum and gastric juice were obtained at each endoscopy for determination of BabA-specific antibodies.

Immunization: Purified BabA (Bugaytsova et al., 2017) (0.50 mg) from H. pylori CCUG 17875 and CTA1-DD (0.05 mg) was prepared in 30 mM sodium phosphate buffer (pH 7.0) with 30 mM ocylglucoside. Each animal was given the vaccine weekly for 4 weeks. The vaccine was administered in a total volume of 1.0 ml, half of which was applied slowly to each of the nares with the animal under ketamine (10 mg/g IM) anaesthesia.

H. pylori challenge: All challenges were performed with rhesus-adapted H. pylori J99, which has a functional Cag PAI, expresses BabA, and attaches to Leb. Bacteria were grown to early log phase in brucella broth with 5% newborn calf serum in 5% CO2. Animals under ketamine anaesthesia (10 mg/kg IM) were oro-gastrically inoculated with 10⁹ cfu.

Endoscopy and quantitative cultures: Animals were fasted overnight and given ketamine anaesthesia (10 mg/kg IM). The gastric antrum was sampled with four biopsies, which were placed in 200 μl of sterile Brucella broth in pre-weighed tubes and homogenized with a sterile glass pestle. An aliquot of 100 μl and dilutions of 10-1 and 10-2 were plated on Brucella agar with 5% newborn calf serum to determine the CFU/mg tissue.

Determination of BabA antibodies in serum by ELISA: Flat-bottom plates were coated with 500 ng purified BabA per well and blocked with skim milk. Serum was diluted 1:50 in PBS containing 0.05% Tween20 (PBST) and incubated in the wells for 1 h at 37° C. Following washing with PBST, goat anti-monkey IgG-AP was added at a dilution of 1:2000 and incubated as above. Antibodies were detected by the addition of p-nitrophenyl phosphate and measurement of absorbance at 415 nm.

### The Leb-Mouse

Maintenance of FVB/N-Leb mice: FVB/N transgenic mice that express human α-1,3/4-fucosyltransferase and thus have Leb-glycosylated gastric epithelium (Leb-mice) (Falk, et al., 1995) were used for this study. Th Leb-mice were kept in an IVC (individual ventilated cage) with no more than four animals per cage at the Umeå Center for Comparative Biology (UCCB), Umeå University. The mice were housed in a 12-h dark/light cycle environment with ad libitum access to commercial diet formula and tap water.

Infection of Leb-mice: Mice were infected at the age of 8-15 weeks. Mice were infected a total of four times for two consecutive weeks by per os gavage with 200 μl of a 50:50 mixture of H. pylori strain USU101 and a 12-month mouse-adapted USU101 output. The infecting inoculum was adjusted by Brucella broth to an approximate dose of 109 CFU/mL (OD 600=1.0).

H. pylori culture for CFUs/g gastric tissue: Stomach samples were transferred into transportation media immediately after sacrifice. Back in the lab, they were vortexed and cultured on selective plates (Brucella agar (BD BBL)-43 g/L supplemented with Iso-Vitox 1% (v/v), 10% citrated bovine blood (Svenska Labfab), 100 μg/ml vancomycin, 20 μg/ml amphotericin B, 10.7 μg/ml nalidixic acid, 200 μg/ml bacitracin, and 3.3 μg/ml polymyxin B). Plates were incubated under microaerophilic conditions at 37° C. for 10 days and examined for bacterial growth. The number of single colonies on the plate was recalculated as CFU/g of gastric stomach tissue sample used for the culture.

### The Leb-Mouse Gastric Cancer Model, Part 1

Sacrifice of the mice and collection of material: The mice were fasted for 2 h prior to sacrifice by cervical dislocation, and blood samples were taken by heart puncture and kept on ice. Serum samples were separated as soon as the samples arrived at the lab by centrifugation for 10 minutes at 11,500×RPM at room temperature and were stored at −80° C. until use. The mouse autopsy started from the first cut to remove the ventral skin. A second cut across the midline opened the abdominal cavity, and two subsequent cuts removed the sternum. Terminal blood was collected by cardiac puncture. The stomach was extracted from the abdomen by cutting off 2-3 mm from the oesophagus to the gastroduodenal junction. Before opening the stomach, the pH values were measured. The stomach was then opened along the long curvature, emptied of its contents, and weighed. The stomach was divided into two parts, including the forestomach, corpus, and antrum. One part was fixed in 4% PFA (HistoLab, Sweden) for histological examination of the tissue. The mucous layer of second part of the stomach was scraped off with a sterile blade and placed into transport medium (2 g casamino acid (Difco), 2 g peptone (VWR), 0.4 g yeast extract (Merck), 0.32 g bacteriological agar (Acumedia), 0.04 g L-cysteine (Merck), 0.2 g glucose, 28 ml glycerol, 1 g sodium chloride (Merck), and 240 mL Milli-Q (Millipore) filtered water (pH 7.0) for quantitative culture.

Gastric pH measurement: The stomach pH was measured in mice fasted for at least 2 h prior to sacrifice using a pH meter (Mettler Toledo Five Easy FE20) with a micro electrode (InLab Micro, Mettler Toledo). Before use, the pH meter was calibrated in the range of pH 2.0 to pH 4.0. To obtain the gastric pH value, the pH electrode was inserted through the pyloric opening in the lumen of stomach without touching the gastric walls. The luminal pH was read when the electrode reached pH stability.

Mouse blood collection: After restraining the mouse, blood was collected by submandibular venous puncture with a lancet (Goldenrod animal lancet, 4 mm). The blood volume never exceeded 0.2 ml.

Mouse serum preparation: The collected whole blood sample was allowed to clot at room temperature for 30 min, and the clot was removed by centrifugation for 11 minutes at 11,500×g. The supernatant, i.e., the serum, was collected in sterile Eppendorf tubes, kept on ice until delivery to the laboratory, and then transferred to a −80° C. freezer.

Histological analysis of Leb-mouse gastric mucosa: Mice were sacrificed by cervical dislocation and their stomachs were dissected through the small curvature. A representative part of the organ with all anatomical regions (forestomach, corpus, and antrum) was placed in a standard histological cassette (Thermo Fisher Scientific, USA) between two biopsy pads (Thermo Fisher Scientific, USA) in order to prevent tissue deformation. Tissue samples were fixed in a 4% neutral paraformaldehyde aqueous solution (HistoLab, Sweden) for 24 h and saturated with paraffin in a Leica ASP300S tissue processor (Leica Microsystems, Germany). Standard paraffin blocks were made with a Leica EG1140 embedding station or a Leica Arkadia H/C system (Leica Microsystems, Germany) with Histowax paraffin (HistoLab, Sweden). Sections with a thickness of 4 μm were cut with a Leica RM2255 automated microtome (Leica Microsystems, Germany), placed on SuperFrost Plus adhesive slides (Thermo Fisher Scientific, USA), and dried overnight at 37° C. Samples were submerged in two portions of xylene for 5 min each for deparaffinization. Rehydration was performed in absolute ethanol (10 min), 96% ethanol (2 min), and 70% ethanol (2 min). Slides were stained with Mayer's hematoxylin and eosin (HistoLab, Sweden), mounted with Pertex mounting medium (HistoLab, Sweden), and dried overnight in a fume hood. All slides were scanned with a Pannoramic 250 Flash Il scanner (3DHistech, Hungary).

Pathological evaluation of gastric disease and inflammation: The hematoxylin and eosin-stained slides contained all anatomical regions of the mouse stomach (forestomach, corpus, and pylorus) and duodenum. Microslides were digitized to full-slide scans with a 250 Flash Ill tissue scanner (3DHistech, Hungary). All samples were analyzed in the 3DHistech SlideViewer (3DHistech, Hungary). Briefly, at least six fields with an area of 1 mm2 of each sample were used for the evaluation. The inflammatory cells (lymphocytes, neutrophils, and macrophages) were counted manually in each field. The mean number of inflammatory cells per field was then graded according to the scale in Example 12. All samples were scored for gastritis, dysplasia, and cancer in situ by a pathologist (R. Mo) in a blinded manner.

### The Leb-Mouse Gastric Cancer Model, Part 2

Identification of the critical age for the Leb-mouse with chronic H. pylori infection to develop dysplasia and/or gastric cancer (FIG. 6, Panel A).

Experimental design: H. pylori-infected mice (60 days old) were sacrificed at different time points to evaluate the gastric cancer incidence and persistence of infection by analysis of stomach biopsies. Mice were sacrificed at 2 months post-infection (n=10), 6 months post-infection (n=30), 9 months post-infection (n=34), and 12 months post-infection (n=10).

### The Leb-Mouse Gastric Cancer Model, Part 3

Identification of the critical age period for the accumulation of the set of mutations that initiate the Correa gastric cancer cascade (FIG. 6, Panel C).

Animal model: For this study, 36 FVB/N Leb transgenic male mice that had been surgically castrated at 6-7 weeks of age were used.

Experimental design: A total of 36 mice were distributed randomly into 4 experimental groups: group 1—infected and treated at 12 weeks post infection, group 2—infected and treated at 22 weeks post infection, group 3—infected and not treated, and group 4—not infected and not treated. All mice were sacrificed at 48 weeks post infection.

Treatment: Mice were treated per os gavage with a mixture of metronidazole, clarithromycin, and omeprazole for 7 days at 12- or 22-weeks post infection according to the experimental set up. No repeated therapy was provided during the whole period of this study. One dose (200 μL) was given per mouse per day consisting of omeprazole (400 μmol/kg, Sigma Aldrich Lot #LRAC0716), metronidazole (14.2 mg/kg, Sigma Aldrich Lot #SLBQ4358V), and clarithromycin (7.15 mg/kg, Sigma Aldrich Lot #019M4018V).

### Vaccination Experiments

Immunization of Leb-mice with BabA, BabB, and SabA antigens: Recombinant proteins were expressed and purified as described by Fei et al., 2011). The vaccination cocktail contained 4 μg (High) or 1 μg (Low) of each recombinantly produced antigen (BabA, BabB, and SabA) and 1 or 5 μg of CTA1-DD (cholera toxin-based adjuvant).

Nasal immunization was performed under 4% isoflurane inhalation anaesthesia with 20 μl of the antigen cocktail (10 μl in each nostril). The vaccination experiment started on week 6 and was performed once a week in two rounds—the first round was at weeks 7-10 after the beginning of the experiment, and the second round was at weeks 17-20 after the beginning of the experiment.

For the first vaccine experiment, 60 FVB/N Leb-mice infected with H. pylori USU 101 were divided into two groups-1) the infected but not vaccinated controls (n=30) and 2) the infected and vaccinated test group (n=30). Mice were immunized with the high dose, 4 μg, of the recombinant vaccination cocktail of BabA, BabB and SabA.

For the second vaccine experiment, 37 FVB/N Leb-mice infected with H. pylori USU 101 were divided into two groups-1) the infected but not vaccinated controls (n=16) and 2) the infected and vaccinated test group (n=21). Mice were immunized with the high dose, 4 μg, of recombinant vaccination cocktail of BabA, BabB and SabA, where Baba was replaced with 6K-BabA), described in Example 19.

For the third vaccine experiment, 89 FVB/N Leb-mice infected with H. pylori USU 101 onco-strain were divided into the following groups: 1) the infected but not vaccinated controls (n=30), 2) the infected and vaccinated test group with the high dose vaccination cocktail (n=27), 3) the infected and vaccinated test group with the low dose vaccination cocktail (n=28) (recombinant vaccination cocktail of 6K-BabA and BabB but without SabA, where BabA was replaced with 6K-BabA, described in Example 19) and 4) the non-infected, non-vaccinated (untouched) controls (n=4).

### Example 10. Induction of bbAbs by Challenge Infection in Human Volunteers and in Rhesus Macaques

Low serum titers of bbAbs against Leb-binding constitute a risk factor for duodenal ulcer disease (FIG. 2, Panels C and D), which suggests the possibility of using immune therapy to boost the levels of such blocking antibodies. To test if experimental H. pylori infection can induce immune responses in terms of blocking antibodies in humans, analysis was performed on sera from 29 volunteers, who had participated in a previous H. pylori vaccination-challenge study and, importantly, had all tested negative for H. pylori at the start of the study by routine diagnostic ELISA, 13C urea breath tests, H. pylori culturing, and histopathological molecular and immunological analysis. Tests of the series of volunteers' sera showed that about 90% (26/29) of the volunteers responded to the experimental infection with the challenge strain H. pylori BCM-300 (of African phylogeny) with high titers of blocking Abs. The inhibitory activity was assessed as the serum dilution at which half the H. pylori binding to Leb was lost, i.e., the 50% Inhibition Titer (IT50). Six volunteers also responded with high broadly blocking Ab activity, i.e., their sera also blocked Leb binding of phylogenetically distant H. pylori such as the 17875/Leb strain. Notably, 5 out 6 of these volunteers induced bbAb that reached or superseded the IT50=approx. 30 level (FIG. 4, Panel A), that can constitute a risk factor for duodenal ulcer disease (FIG. 2, Panel C). Unexpectedly, however, four individuals demonstrated pre-challenge titers, which might reflect ongoing but undetected low-level H. pylori infections as reported by the sensitive 16S RNA sequencing protocols 16 (FIG. 4, Panel A). These results show (1) that humans can respond to a challenge infection with induction of protective levels of bbAbs, (2) that the bbAbs against the experimental infection are similar to the majority of individuals and subject cohorts world-wide who carry H. pylori infections, and (3) that the pre-challenge titer results suggest that the IT50 test of a blood sample collected by a finger stick can identify those individuals who are likely to carry non-detectable infections, in this case 14% (4/29) of H. pylori-“negative” individuals.

The positive results prompted us to test if experimental H. pylori infection can similarly induce bbAb responses in rhesus macaques, i.e., in a primate model. Based on a recent study on BabA adaptation during H. pylori infection in rhesus macaques, the inventors analyzed sera from five specific pathogen-free (SPF) animals. The five animals had all tested negative for H. pylori infection, routine diagnostic ELISA, H. pylori culturing and histopathology, and they had subsequently been challenge-infected with the H. pylori J166 strain.

Sera from 14- and 20-weeks post-infection showed that three of the animals demonstrated high IT50s, whereas two animals exhibited low IT50s, when tested using J166. Unexpectedly, the three animals with high IT50s also demonstrated similarly high pre-challenge titers (FIG. 4, Panel B).

These results suggest that these three SPF animals carried non-detectable H. pylori infections already at the start of the experiment (FIG. 4, Panel B). Notably, the two SPF animals that responded with IT50=about 200 when tested with the J166 challenge strain also tested positive with IT50=about 50 when tested with the non-related Indian strain 19 (FIG. 4, Panel Bii)

In contrast the Leb-high affinity binding strains 17875/Leb did not manage to detect IT50 titers in these two animals. Thus, the Leb-Low-affinity binding strains such as H. pylori J166 and 19 are more efficient in detecting H. pylori infection, compared to the Leb-high-affinity binding strains such as 17875/Leb.

Sera from the 14- and 20-weeks post-infection also showed that three of the animals demonstrated high IT50s, when tested using J166. Unexpectedly, the three animals with high IT50s also demonstrated similarly high pre-challenge titers. These results suggest that these three SPF animals carried non-detectable H. pylori infections already at the start of the experiment (FIG. 4, Panel Bi), i.e. chronic infections that could be detected by the presence of IT50 in their serum samples.

The tests demonstrated that H. pylori infection can be detected by the broadly blocking antibodies (bbAb) produced. Thus, these bbAb-antibody responses i.e., serum samples, can be used as a sensitive diagnostic biomarker to detect H. pylori infection in both humans and rhesus macaques (FIG. 4, Panel Ai and 4, Panel Bi).

The animals no. 2 and 3 were free of H. pylori infection before start of test and were then infected by strain J166. At 14- and 20-weeks post infection they had responded with bbAb antibodies as detected by the Low-affinity-binding strains J166 (FIG. 4, Panel Bi) and 19 (FIG. 4, Panel Bii). In contrast the induced IT50 signal of the animals no. 2 and 3 could not be detected by the Leb-high-affinity binding strain 17875.

### Example 11. Induction of bbAb Responses by Vaccination with BabA Protein in Rhesus Macaques

To test if vaccination of primates with BabA as a vaccine antigen can elicit a protective immune response against an H. pylori challenge infection, the inventors selected a group of nine SPF rhesus macaques that all tested negative for H. pylori infection by routine diagnostic ELISA, H. pylori culturing and histopathology. The five animals were immunized intranasally once a week for 4 weeks with a vaccine composed of the native BabA protein purified from H. pylori 17875 (Bugaytsova et al., 2017) and the mucosal adjuvant CTA1-DD (Ågren, et al., 1998, Lycke and Schon, 2001). The immune response in serum was tested by ELISA at 4 weeks post-vaccination and showed induction of BabA antibodies in 4 of 5 vaccinated animals (FIG. 5, Panel Bi).

At the 4 weeks post-immunization time point, all nine animals were challenged with H. pylori strain J99, a reference strain of African phylogeny and hence phylogenetically distant from the 17875 strain of European phylogeny, which was the source of the BabA vaccine antigen). The J99 strain infected all animals to similar high H. pylori load as is common in humans and rhesus macaques (Hansen, L. M. et al., 2017; and Atherton, J. C. et al., 1996).

Two of the five BabA-vaccinated animals demonstrated reduced infectious loads at 2 weeks after infection, and both “cleared” the infection at 4 weeks. However, at 8 weeks the J99 infection returned although with a two log-fold reduced infectious load as compared to the other three animals (FIG. 5, Panel Bii). The two animals that responded with a lowered infectious load also demonstrated the strongest ELISA signal FIG. 5, Panel Bi) These results suggest that the prophylactic vaccination does not protect from infection but that the BabA-ELISA immune response is associated with a temporal reduction of the H. pylori infectious load.

Next, the serum samples were tested for vaccination-induced IT50 response of bbAb activity. Two animals from the vaccinated group and two animals from the control group demonstrated stable pre-existing IT50s that ranged from 1,000 to almost 100,000, suggesting that they carried a non-detectable H. pylori infection already at the start of the experimental series FIG. 5, Panel D).

In contrast, the other animals in each group demonstrated no IT50 pre-vaccination titers. Instead, these vaccinated animals responded with an IT50=approx. 25 at 8 weeks post-challenge by strain J99 and tested by the phylogenetically distant strain 17875 FIG. 5, Panel C). Notably, testing with the phylogenetically distant Indian strain 19 demonstrated that the animals had a broadly blocking response with an IT50=˜100 FIG. 5, Panel C). The results demonstrate that BabA vaccination can induce bbAb responses with a potential to protect against gastric disease. The many SPF animals with pre-challenge IT50 titers suggests that H. pylori can maintain stable long-term BabA expression in rhesus macaques. This might contradict previous reports that H. pylori infection in rhesus macaques is accompanied by rapid loss of BabA expression due to loss of the babA gene.

However, the two scenarios are principally different because rhesus macaques and humans exhibit similar high infectious loads of H. pylori in the range of 10⁵-10⁸ bacteria/gram of gastric tissue. In comparison, the infectious loads in the SPF animals would be in the range of <10²/gram i.e., thousands or even a million-fold lower and hence non-detectable with routine diagnostic tools, nor by culture.

From this it can be concluded (1) that measuring IT50s using the 1251-Leb-competition technique is a considerably more sensitive method compared to routine H. pylori diagnostics; (2) that SPF animals can carry natural H. pylori infections; (3) and that the very small infectious loads with no signs of gastric inflammation might support a lifestyle of “humanized” tissue-tropism for H. pylori with expression of BabA. (4) that the prophylactic vaccination does not protect from H. pylori infection (animals V2, V3 and V4): (5) natural high levels of blocking antibodies (IT50) do not protect from H. pylori infection (animals V1, V5, A2 and A3): (6) however, the BabA-vaccination induced IT50≥30, which can constitute a protective level of bbAbs against gastric disease (according to the results on the IT50=30 Risk factor level in FIGS. 2, Panel E and 2, Panel F).

### Example 12. a Mouse Model of Gastric Cancer Caused by Long-Term Chronic H. pylori Infection with Long-Term Stability of BabA-Mediated Leb-Binding Activity

The inventors developed a mouse model for H. pylori-induced gastric cancer. The cancer model is based on the Leb-mouse that expresses a gastric epithelium with humanized Leb-glycosylation, which supports BabA-mediated H. pylori attachment (Falk et al., 1995). and gastric mucosal inflammation (Moonens et al., 2016) and thus is potentially applicable as a gastric cancer model (FIG. 6). The Leb-mouse cancer model was developed in a series of steps. First, chronic infection over 12 months by the H. pylori onco-strain USU101 resulted in a notably high incidence of gastric cancer of 56% and an additional 18% incidence of dysplasia, i.e., 74% (25/34) of the animals (Group I) developed severe gastric disease with a mean inflammation score of 2.25 (scale 0-3) (FIG. 6, Panel B). Second, and in sharp contrast, none of the 12 non-infected control Leb-mice (Group III) developed gastric cancer, dysplasia, or inflammation (all scored 0) during the 12-month period (FIG. 6, Panel B).

Thus, gastric cancer and chronic inflammation are entirely dependent on the H. pylori infection in the Leb-mouse cancer model. Third, the results were followed up by a second 12-month infection test (FIG. 6, Panel B), which presented a 40% (12/30) incidence of gastric cancer and an additional 27% (8/30) incidence of dysplasia, i.e., 67% (20/30) of the mice developed malignant gastric disease with a mean inflammation score of 1.4 (FIG. 6, Panel B). Thus, the gastric cancer and dysplasia prevalence was reproducible in the second 12-month test, albeit with a slower transition of the dysplasia into gastric cancer, which is also reflected in the lower mean inflammatory score, i.e., 2.25 vs. 1.4, respectively.

The gastric mucosal inflammatory infiltration scores were as follows:
Grade 0: The mucous membrane and submucous plate contain rare inflammatory cells, which are scattered throughout the tissue. Inflammatory cells do not form piles (infiltrations) or groups. There are fewer than 20 inflammatory cells (neutrophils, lymphocytes, macrophages, and plasmocytes) visible in the field of view (FOV) (diameter: 1000 μm).
Grade 1: The inflammatory cells are located in the basal part of the mucous membrane and submucous plate where they can form small groups or piles. There are 20-100 inflammatory cells (neutrophils, lymphocytes, macrophages, and plasmocytes) visible in the FOV.
Grade 2: Inflammatory cells are seen in all parts of the mucous membrane and submucous plate. Inflammatory infiltrates (like small lymph nodules) are seen in the basal part of the mucous membrane and submucous plate. The gastric tissue contains 100-300 inflammatory cells per FOV. In addition, the tissue exhibits fulminant gastric adenocarcinoma with growth and penetration through the submucosal layer (lamina propria indicated by green arrows) and with the invasion of cancer tissue (green box) into the muscular layer (indicated by the black arrows).
Grade 3: The inflammatory infiltrates are present in all layers of the gastric tissue and form large inflammatory infiltrates (similar to lymphatic nodes) in the submucous plate (between the muscular and mucus layers). The gastric tissue contains more than 300 inflammatory cells in the FOV.

All H. pylori-infected mice (Groups I and II) demonstrated gastritis at 12 months, which suggests that the take-up of the H. pylori infection was approx. 100%. At the 12-month end-point, 47% (16/34) of the Group I mice and 30% (9/30) of the Group Il mice still carried H. pylori infections that were detectable by culture (FIG. 6, Panel D). Unexpectedly, only one out of the 64 infected mice in Groups I and II responded with detectable IT50s during the 12 months of H. pylori infection. This concludes that the absence of IT50s of blocking antibodies in mice in response to chronic H. pylori infection is very different from the IT50 responses in humans and macaques. The lack of protective IT50s of blocking antibodies in mice against the H. pylori onco-strain USU101 seems to be a critical limitation in the Leb-mouse immune system, with resulting excessive chronic mucosal inflammation and an extraordinarily high incidence of gastric cancer and dysplasia.

Identification of the critical age for the Leb-mouse with chronic H. pylori infection to develop dysplasia and/or gastric cancer. To determine the critical age period for establishing gastric cancer in the Leb-mouse model, the animals were scored after 2, 6, 9, and 12 months of chronic infection with the onco-strain USU101. The incidence of dysplasia increased from 6 months after infection, whereas the incidence of gastric cancer increased from 10% at 9 months to 30% at 12 months (FIG. 6, Panel A). These results show that the development of gastric malignancies in the Leb-mouse model follows the Correa cascade over the lifetime of chronic H. pylori infection to the final destination, gastric cancer.

Identification of the critical age period for the accumulation of the set of mutations that initiate the Correa gastric cancer cascade. The inventors subsequently determined the period in life when the chronic H. pylori infection fuels the mucosal inflammatory processes with a critical build-up of mutations that eventually initiates the Correa cancer cascade. For this test, three groups of Leb-mice where infected with the onco-strain USU101. After 12 and 22 weeks the infections were eradicated by antibiotics, whereas the third group was left untreated i.e., with life-long 48 weeks of H. pylori infection. The 22-week antibiotic-treated and the 48-week non-treated mice both demonstrated a high (70%) incidence of malignant cell development, i.e., gastric cancer and dysplasia (FIG. 6, Panel C). These results suggest that the 10 weeks infection period, weeks 12 to 22, is critical for accumulation of mutations. These results argue for early intervention in protecting against excessive chronic inflammation because the Correa gastric cancer cascade is the consequence of mutations that occurred earlier in life.

Long-term stability of both the chronic H. pylori infection and BabA-mediated Leb-binding activity. Within Group Il and the non-antibiotic-treated group (the 48 weeks infection group), only 30% and 20% of the mice carried H. pylori at 12 months, respectively. A low prevalence of H. pylori infection in subjects with gastric cancer is well described (Parsonnet, J. et al., 1991) and is a consequence of the dramatic changes in the gastric environment due to loss of the acid-secreting parietal cells and the resulting rise in gastric pH.

To test the stability of H. pylori infection during chronic infection, the inventors first analysed the groups of mice infected with onco-strain USUS101, and after 2, 6 and 9 months they demonstrated 90% (9/10), 83% (25/30), and 59% (20/34) infection rates, respectively (FIG. 6, Panel D). At the 12 months endpoint, the 4th group demonstrated the expected reduction of 40% (4/10) infection prevalence, which was similar to the collective median (38%) of the five different 12-month infections (120 mice) shown here (FIG. 6, Panel D). Thus, it can be concluded that during the critical period of accumulation of stem-cell mutations, i.e., the first 3-5 months of the experiment, the vast majority of the Leb-mice carry stable H. pylori infections.

Second, to test for the retained prevalence of BabA-mediated Leb binding activity, the inventors tested the mice that carried H. pylori at 2, 6, and 9 months, and approx. 80% demonstrated retained BabA-mediated Leb-binding activity (FIG. 6, Panel E). However, at 12 months, i.e., at the Correa cascade endpoint, the majority (approx. 75%) of the H. pylori infections in the mice had lost their Leb-binding activity (FIG. 6, Panel E). Thus, approx. 70% of the Leb-mice carried H. pylori infections that expressed BabA-mediated Leb-binding activity during their first 6 months of life, i.e., the window in time when the critical mutations accumulate that drive the Correa gastric cancer cascade.

### Example 13. The First Vaccine Experiment: Induction of bbAb Response by Vaccination and Protection Against Gastric Cancer

The present results show that vaccination with BabA induces IT50s of bbAb activity in rhesus macaques (FIG. 5). However, for the design of a general vaccine composition the inventors took into consideration that long-term H. pylori infections in rhesus macaques, mice, and gerbils can reduce the expression of BabA and upregulate the closely related BabB paralog that has an as yet unknown function. In comparison, the strong 84% stability in Leb binding over 6 months of H. pylori infection by the onco-strain USU101 in the Leb-mouse (FIG. 6, Panel E) might be a consequence of its “humanized” Leb-glycosylated gastric mucosa that supports BabA-mediated adherence of the H. pylori infection. By so doing, the “humanized” Leb gastric mucosa makes the Leb-mouse suitable for vaccine studies. Thus, to test if vaccination can induce protective IT50s, the inventors combined the major H. pylori adhesin proteins BabA, BabB, and SabA, recombinantly expressed with C-terminal Myc- and Histidine-tags (peptide sequence EQKLISEEDLSHHHHHH) (in FIG. 11) as described by Fei et al., 2011), with the mucosal adjuvant CTA1-DD into a vaccine composition.

A group of 30 Leb-mice were therapeutically vaccinated one month after established infection by the onco-strain USU101. At the 12-month endpoint, both the total incidence of malignant cell development and the inflammation scores were similar in the vaccinated and non-vaccinated (control) mice (77% vs. 67% cancer rates and 1.5 vs. 1.4 inflammation scores, respectively) (FIG. 7, Panel A). However, the IT50 responses were very differently distributed in the vaccinated group (FIG. 7, Panel B), where the HIGH IT50 mice demonstrated a approx. 6-fold reduced incidence of gastric cancer (p<0.038*) and a approx. 9-fold reduction in total dysplasia and cancer (p<0.033*) (FIG. 7, Panel C). The HIGH IT50 mice also scored low (1.0) and were protected from inflammatory infiltration in contrast to the LOW IT50 mice and the non-vaccinated mice, which all scored high for inflammation (LOW IT50, 1.7; Group 1, 2.25; and Group II, 1.4) (FIG. 7, Panel C).

The high levels of inflammation in the mice with low IT50s is explained by the very strong correlation between high levels of inflammation and gastric cancer (FIG. 7, Panel D), i.e., it is very similar to the high inflammatory infiltration seen in human gastric cancer. Tests by immunoblots showed that only the serum antibodies from the HIGH IT50 mice recognized structural epitopes in BabA, whereas sera from all LOW IT50 and HIGH IT50 vaccinated mice recognized linear epitopes in both BabA and BabB to the same extent, and vice versa, where the non-vaccinated animals' sera showed no BabA/B reactivity at all. Next, H. pylori was grown from biopsies to understand if the vaccine could provide eradication and sterile clearance of the infection. However, the prevalence of vaccinated mice with LOW IT50 or HIGH IT50 that carried H. pylori infection at 12 months were similar in the two groups (9/23=39% vs. 2/7=28%, and 36% in total).

The first therapeutic vaccination pilot experiment suggests (1) that the therapeutic vaccination induces IT50s that reduce gastric mucosal inflammation and gastric cancer, (2) that the non-vaccinated Leb-mice do not respond with protective IT50s during the 12 months of chronic H. pylori infection, (3) that the therapeutic vaccination does not cause sterile clearance of the H. pylori infection, and (4) that only the protective serum antibodies from the HIGH IT50 mice recognize structural epitopes in BabA.

### Example 14. The Second Vaccine Experiment: Induction of bbAb Response by Vaccination and Protection Against Gastric Cancer

We next performed a second vaccination experiment aimed at increasing the prevalence of HIGH IT50 responders. Thus, mice with an established H. pylori infection were therapeutically vaccinated with freshly prepared recombinant BabA, BabB, and SabA antigens. This time, all mice responded to vaccination and with 4-fold higher median IT50 levels compared to the previous HIGH IT50 mouse group (FIG. 8, Panel C). Notably, all 21 of the vaccinated mice were protected from gastric cancer, whereas 30% (5/16) of the non-vaccinated mice developed gastric cancer (Fisher: p<0.01**) (FIG. 8, Panel B). The vaccine also reduced the inflammatory infiltration from 1.5 down to 1.05 (Wilcoxon: p=0.03*) (FIG. 9, Panel A and 12).

Again, the inflammatory infiltration correlated with the severity of disease, and the mice with gastritis had a mean score of 1.1 compared to a score of 2.2 in those with gastric cancer (p<0.001**). The non-vaccinated mice with dysplasia scored higher (1.67) compared to the gastritis group (Dunn's test; p=0.039). In contrast, the vaccinated mice with dysplasia scored only 1.2, which was similar to the score of 1.1 for the gastritis group. The number of vaccinated mice that carried an H. pylori infection at 12 months was identical to the non-vaccinated group at 8/21 and 6/16, respectively, i.e., 38% in both groups, and thus essentially identical to the first pilot vaccination experiment (36%) and similar to Group I (47%), Group II (30%), and the 2-12-month test (40%) (FIG. 6, Panel D).

The second mouse vaccination experiment demonstrated that the therapeutic vaccination induces high IT50s in 100% of the vaccinated animals, which reduces gastric inflammation and protects against gastric cancer despite not eradicating the H. pylori infection.

### Example 15. The Third Vaccine Experiment: Induction of bbAb Response by Vaccination and Protection Against Gastric Cancer

The promising results presented above warranted a combined test of dose-response and reproducibility. Mice were again therapeutically vaccinated, but with High (vaccine-1) or Low (vaccine-2) BabA/BabB antigen levels combined with 5-fold more CTA1-DD adjuvant compared to the previous series. The mice were scored at the 12-month end-point, and the median IT50s were high in both groups and were similar to the second vaccine experiment and were only 2-fold lower in the Low-antigen group. The vaccine fully protected against gastric cancer, compared to the 17% (5/30) gastric cancer incidence in the non-vaccinated mice (p<0.004**) (FIG. 9, Panel D and 13).

Furthermore, the non-vaccinated group had 57% (16/30) mice with dysplasia, compared to 17% (9/55) of the vaccinated animals (p<0.0005***) (FIG. 9, Panel D and 13). The High and Low antigen vaccine groups presented 26% (7/27) vs. 7% (2/28) dysplasia, respectively, indicating that a combined high dose of both antigen and adjuvant might be a less protective vaccine composition against malignant development. Notably, the vaccine also protected against total gastric malignant development, with 70% (16 dysplasia+5 gastric cancer/30) malignancy in the non-vaccinated group compared to only 17% (9 dysplasia/55) in the vaccine groups (p<0.0001***).

In this third vaccine experiment, the Correa cascade developed somewhat slower with 17% (5/30) gastric cancer cases in the non-vaccinated mice compared to 30% (5/16) in the second vaccine experiment. The slower cascade was also reflected in the proportionally higher incidence of dysplasia of 53% (16/30) compared to the 19% incidence in the second vaccine experiment. The lower gastric cancer incidence might be a consequence of the lower mucosal inflammation level, 1.25, compared to the higher level of 1.5 in the second vaccine experiment test (FIG. 9, Panel A).

The combined cancer reduction in the second and third vaccine experiments, i.e., 5+5 cancers in the 46 non-vaccinated mice vs. no gastric cancer cases in the 76 vaccinated mice, indicates that the vaccine is highly protective against cancer (p<0.001***) (FIG. 9, Panel F).

### Example 16. Vaccination Protected Against Loss of Gastric Juice Acidity and the Induced IT50s are Present in the Gastric Tissue

It is well recognized that subjects with gastric cancer demonstrate elevated gastric pH due to atrophic gastritis and loss or reduction of the acid-producing parietal cells, which are pathognomonic events in the Correa cascade. To test for similar changes, the gastric pH of the Leb-mice was measured at the 12-month endpoint. The inventors found that the gastric pH was surprisingly low in both the healthy-infected-control and healthy-infected-vaccinated mice, pH 1.4 and 1.7, respectively, and that it increased to pH 2.8 in the mice with gastric cancer (p=0.033*).

The non-vaccinated mice with dysplasia had an increase to pH 2.37, whereas the vaccinated animals were protected against the loss of gastric acidity (p=0.014*). Similarly, in the third vaccine experiment the non-vaccinated mice demonstrated elevated gastric pH compared to the vaccinated group (p=0.0007***) (FIG. 9, Panel D).

### Example 17. Vaccination Induced bbAb Activity

To test if the vaccination in addition to blocking antibodies also induced responses of protective bbAbs in the Leb-mice, i.e., similar to the bbAb responses in the vaccinated rhesus macaques (FIG. 5, Panel C), the sera of the 21 vaccinated mice were tested for IT50s with a full series of 13 global H. pylori isolates, including the challenge onco-strain USU101. The serum samples from the vaccinated animals inhibited Leb binding of the full series of H. pylori strains in contrast to non-vaccinated controls. The vaccine induced a strong immune response of bbAb activity with a median IT50=approx. 200 (with the antigen source, the 17875/Leb strain, excluded) (FIG. 10, Panel A).

Thus, the mice responded similar to humans, with generally high IT50 titers of bbAbs against Leb binding with the series of global H. pylori infections, including the challenge onco-strain USU101. The results on the general and broadly blocking antibody responses help explain why the BabA vaccine efficiently protects against gastric cancer caused by the phylogenetically distant challenge onco-strain USU101.

### Example 18. Vaccine Induced IT50s of bbAbs Against H. pylori Attachment to the Gastric Mucosa

The reduced mucosal inflammatory infiltration in the vaccinated animals suggests that the vaccine responses block and reduce H. pylori adherence to the gastric mucosa. In support of this notion, the pooled sera of vaccinated mice almost completely (95%) blocked attachment to human gastric mucosa by the strain 17875/Leb. To further understand if the vaccine-induced IT50s also support broadly blocking protection against H. pylori attachment to the gastric mucosa, the challenge onco-strain USU101 was exposed to the pooled sera of vaccinated mice. Indeed, the 10-fold dilution of the serum reduced attachment of the mouse model challenge strain USU101 to human gastric mucosa by 80%.

To further understand if the vaccine-induced IT50s also provide broadly blocking of H. pylori attachment, we exposed the pooled serum samples from the 2nd vaccination to the series of 11 global H. pylori isolates, including the challenge onco-strain USU101. Indeed, the serum reduced attachment of all 11 isolates from Europe, Africa, Asia, and Americas by ˜80% (FIG. 10, Panel B). The strong and broadly blocking of H. pylori attachment to human gastric mucosa, suggests that the BabA-vaccine holds promises for global protection against gastric disease.

### Example 19. Vaccination with Structurally Stabilized BabA Antigen in Protection Against Gastric Cancer

In the first mouse vaccination experiment the mice were vaccinated with the H. pylori BabA, BabB, and SabA protein mixed with the mucosal adjuvant CTA1-DD into a vaccine composition. A group of 30 Leb-mice were therapeutically vaccinated one month after established infection by the onco-strain USU101. At the 12-month endpoint, the IT50 responses were very differently distributed in the vaccinated group, where the series of IT50 titers differed several log-fold, from mice with no induction in IT50 to mice with IT50>1000-20.000. The 30 immunized mice presented in increasing order of IT50, which made a natural divider of the two groups at IT50=approx. 1000 i.e., LOW IT50 vs. HIGH IT50 mice (FIG. 7, Panel B). Especially so since the HIGH IT50 mice demonstrated an about 6-fold reduced incidence of gastric cancer (p<0.038*) and an about 9-fold reduction in total dysplasia and cancer (p<0.033*) (FIG. 7, Panel C). The HIGH IT50 mice also scored low (1.0) and were protected from inflammatory infiltration in contrast to the LOW IT50 mice and the non-vaccinated mice, which all scored high for inflammation (LOW IT50, 1.7; Group 1, 2.25; and Group II, 1.4) (FIGS. 7, Panel A and 7, Panel B).

To improve on the efficacy of the vaccine antigen, the inventors designed the BabA protein with a C-terminal hexa-lysine-peptide, denoted 6K-BabA (polypeptide sequences shown in FIG. 26. Positively charged peptides, such as hexa-Lysine and hexa-Arginine and derivatives of their lengths (shorter or more extended), have been described to improve the solubility and yield of many different recombinantly expressed proteins (Kato et al., 2007; Paraskevopoulou et al., 2022), including BabA (Hage et al., 2014) and additional H. pylori proteins (Paraskevopoulou et al., 2019), suggesting increased resistance against proteases and degradation during expression in non-H. pylori systems, such as the common E. coli recombinant expression system.

Thus, the BabA from strain 17875 was expressed with and without the hexalysine peptide and tested for its ability to form crystals and to provide 3D structure information by diffraction analyses. Most interesting, the inventors found that the BabA protein with the hexa-lysine peptide formed ordered crystals (PDB 8R4P/rcsb.org/structure/unreleased/8R4P)) in contrast to the BabA without hexa-lysine peptide suggesting that the structure was more structurally/3D-stable, which allowed for the BabA protein to be organized into a crystal. In contrast, the recombinant BabA protein lacking the hexa-lysine peptide did not crystallize (data not shown). The inventors have previously shown that the 17875BabA protein (without the hexalysine peptide) needs be in complex with stabilizing nano-bodies (produced by immunization of lama animals) to crystallize for diffraction information (Moonens et al., 2016).

The present results suggest that the hexa-lysine peptide does not merely improve the recombinant yield of protein by protecting the BabA protein against degradation and proteases and with increase thermal stability, but also helps reconstitute the authentic structural stability the BabA protein to allow for crystallization. The inventors suggest that the improved structural stability of the BabA protein might also involve the BabA carbohydrate binding domain (CBD), where the ABO/Leb glycans bind but where also the broadly blocking antibodies bind through the mechanism of Glycan mimicry (FIG. 3, Panel D).

Thus, during the humoral immune response cascades with antigen presentation for induction of antibody producing B-cells, the present inventors postulated that the a vaccine antigen, such as the BabA protein, equipped with increased structural stability and hence better fit for the ABO/Leb glycan, will induce an antibody response that would bind to the structural epitopes of the (BabA) antigen with a corresponding tighter fit and hence higher binding strength. Such enhanced binding strength (affinity) of the antibodies would be reflected in the higher IT50 titers by vaccination with the hexa-lysine modified vaccine antigen, such as BabA, and hence better protection against disease.

Next, a series of mice were immunized with the recombinant BabA protein, i.e., BabA protein with or without the C-terminal hexa-lysine-peptide combined with the mucosal adjuvant CTA1-DD. The animals were immunized similarly to the previous described vaccination series by mucosal vaccination, applied once per week for 4 weeks, with one month for induction of initial immune response, followed by a similar series of mucosal vaccinations, applied once per week for 4 weeks. The sera samples were collected one month after the last immunization and tested for induced IT50 titer responses. The mice immunized with 1 micro-gr hexa-lysine BabA (6K-BabA) demonstrated a median IT50=13609, compared to IT50=8006, by 1 micro-gr of BabA without hexalysine. Thus, the 6K-BabA indeed and surprisingly demonstrated 70% increase in IT50 compared to the BabA lacking the hexa-lysine-peptide. The 6K-BabA demonstrated a mean IT50=14787 compared to IT50=10400 by 1 micro-gr of BabA without hexalysine. Thus, the 6K-BabA demonstrated 40% increase in mean IT50 compared to the BabA antigen lacking the hexa-lysine-peptide. For comparison, since high levels of vaccination antigen might saturate the immune system and antigen presenting cells, in parallel the inventors immunized with 5-fold lower level of BabA antigen. The mice immunized with merely 0.2 micro-gr hexa-lysine BabA (6K-BabA) demonstrated the median IT50=87 compared to IT50=22, by 0.2 micro-gr of BabA without hexalysine. Thus, the 6K-BabA demonstrated 4-fold increase in median IT50 compared to the BabA lacking the hexa-lysine-peptide. In addition, the 6K-BabA titers demonstrated a mean of IT50=949 compared to merely IT50=175 for BabA. Thus, the 6K-BabA demonstrated >5-fold significant increase in mean IT50 compared to the BabA lacking the hexa-lysine-peptide (FIG. 8, Panel A). The test showed that the mice responded with higher IT50 titers to the hexa-lysine tag mediated improved structural stabilization.

To test the 6k-BabA antigen for improved structural stability and hence increased binding affinity for the cognate ligand, i.e., the ABO/Leb antigens, the innovators performed structural/functional tests of the 6K-BabA by application of a serial dilution of recBabA and rec6KBabA. The dilution were applied to Leb-antigen that was coated and hence immobilized in ELISA plates. The results demonstrated that rec6K-BabA binds the Leb antigen with several log-fold higher binding strength compared to BabA. The huge difference in binding affinity argues for that the 6KBabA has a greatly improved structural stabilization of its carbohydrate binding domain (CBD), compared to recBabA (FIG. 8, Panel B).

The two tests showed that the mice responded with higher IT50 titers to the BabA antigen equipped with the hexa-lysine (6K) tag, a modification that drastically improves its structural stabilization. Thus, the new results suggest that the increased structural stability of the antigen contributes to enhanced immune response with higher IT50 titers of protective antibodies that bind to structural antigen epitopes.

The inventors next performed a second full vaccination experiment aimed at increasing the prevalence of HIGH IT50 responders. Thus, mice with an established H. pylori infection were therapeutically vaccinated with freshly prepared recombinant hexa-lysine BabA, combined with recBabB recSabA antigens and CTA1-DD adjuvant. By this optimized vaccine composition combination all mice responded to vaccination with IT50 titers and, in addition, with 4-fold higher median IT50 levels (IT50=7667 (FIG. 8, Panel C), ie the 2nd Vaccine Vaccinated)) compared to the previous HIGH IT50 mouse group (IT50=1813 (FIG. 7, Panel B)). Notably, all 21 of the vaccinated mice were protected from gastric cancer, whereas 30% (5/16) of the non-vaccinated mice developed gastric cancer (Fisher: p<0.01**) (FIG. 8, Panel D). The results suggest that the hexa-lysine peptide tagged vaccine antigen induce general IT50 responses in the vaccinated mice, i.e. 1) all mice respond with IT50 titers, compared to merely 25% in the 1st vaccine experiment (FIG. 7, Panel B vs. 8, Panel C); 2) with ˜4-fold increased IT50 titers compared to the 1st vaccine experiment (FIG. 7, Panel B vs. 8, Panel C); 3) with resulting full protection (FIG. 8, Panel D) compared to partial protection (FIG. 7, Panel C) against gastric cancer.

The promising results with hexa-lysine modified BabA warranted a third vaccine experiment, with a combined test of dose-response and reproducibility. Mice were again therapeutically vaccinated, but with high (Vaccine-1, 4 μg of 6K-BabA) or low (Vaccine-2, 1 μg 6K-BabA) antigen levels combined with recombinant BabB (recBabB) and CTA1-DD adjuvant and in addition, a third group, Group 3 with 1 μg 6K-BabA antigen and recBabA but with no CTA1-DD adjuvant (FIG. 9, Panel B). The reduced level of BabA antigen (with adjuvant) would be expected to generate the correspondingly lower IT50 response, and the Group 3 mice without adjuvant would likely respond to BabA vaccination with even lower IT50 levels.

The mice were scored at the 12-month endpoint, and the median IT50s were high in both Vaccine-1 and Vaccine-2 groups. The Vaccine-1 group (with 4 μg 6K-BabA) was most similar to the second vaccine experiment in median IT50 (7026 vs 7667) whereas the Vaccine-2 group with merely 1 μg 6K-BabaA was 2-fold lower in median IT50=3557 (FIG. 8, Panel C). Importantly, none of these pooled mice sera groups demonstrated IT50s titers below IT50=2500. Both the Vaccine-1 and Vaccine-2 protected against gastric cancer, compared to the 17% (5/30) gastric cancer incidence in the non-vaccinated mice (p<0.004**). Furthermore, the non-vaccinated group had 57% (16/30) mice with dysplasia, compared to 17% (9/55) of the vaccinated animals (p<0.0005***) (FIG. 9, Panel B). Notably, the Vaccine- and Vaccine-2 also protected against total gastric malignant development, with 70% (16 dysplasia+5 gastric cancer/30) malignancy in the non-vaccinated group compared to only 17% (9 dysplasia/55) in the vaccine groups. Analyze by use of a 2×2 contingency table, demonstrates that the 6K-BabA-vaccine raised full immuno-protection against gastric cancer (p<0.0001***) (FIG. 9, Panel C).

In comparison, the Group 3 which was vaccinated without adjuvant, responded with the low median IT50=1695, i.e. two-fold lower compared to the Vaccine-2 group, which was similarly vaccinated with 1 μg 6K-BabaA, the difference was that there was no CTA1-DD adjuvant in the vaccine for the Group 3 (FIG. 8, Panel C and 9, Panel B).

The median IT50=1696 is close to the critical IT50 titer=1000 which separated the LOW and HIGH IT50 mice in the first vaccine experimental series (FIG. 7, Panel B). Albeit merely 2-fold lower compared to the Vaccine-2 series, IT50=1695 of Vaccine-3, group 3) vs. IT50=3557 (Vaccine-2, group 3), (FIG. 8, Panel C and 9, Panel B). the Group 3 (without adjuvant) exhibited booth gastric cancer (1 case, vs. 0 case in the Vaccine-2 group) and high level of dysplasia (15 cases out of 28 animal vs. 2 dysplasia cases in Vaccine 2 group). The 15 cases in the Group 3 are most similar to the 16 cases in the non-vaccinated control group. Thus, the 2-fold lower median IT50 level in Group 3 did not mobilize enough of humoral immune protection. Instead, the IT50 titers are too low and accumulates close to the critical level for gastric cancer protection in the mouse model, i.e., IT50=˜ 1000 (FIG. 7, Panel B). The 2-fold lower median IT50 level is Group 3 is also reflected in the mucosal inflammation level, 1.22, which is most similar to the 1.25 score in non-vaccinated controls and hence higher compared to the vaccinated animal in the Vaccine-1 and Vaccine 2 groups of mice.

The results point to the importance of using vaccine compositions that can induce protective levels of IT50 titers. For protective immune responses with high iT50 titers, a high degree of structural stability is essential for the BabA protein antigen. The structural stability of different BabA protein from different clinical isolates probably varies, as a reflection of the several log-fold differences in ABO/Leb binding affinities among clinical isolates (Aspholm, Science, 2004). This is also reflected in the two ABO/Leb-high affinity binding strains 17875 and J99, which are the only strains that express BabA protein which has generated BabA protein crystals and 3D structural diffraction patterns. The experimental immunization series with hexa-lysine modified 6K-BabA, demonstrated that the structurally stabilized BabA antigen can induce several-fold higher IT50 titers. The present results with the 2-fold lower median IT50 titers generated by the vaccine composition devoid of adjuvant shows that a 2 to 4-fold higher IT50 can make a critical and important difference in protection against gastric cancer.

The results suggest that there is a need for much higher IT50 titers to protect mice from gastric cancer compared to the risk factor IT50 level proposed as a protective level against gastric disease such as Duodenal Ulcer disease (DU). In the gastric cancer mouse model, the critical IT50 level is close to 1000. In the first vaccine experimental series, 75% of the mice did not respond with IT50>1000 (LOW) and were consequently not protected against gastric cancer in contrast to the 25% of HIGH responding mice.

To improve on the BabA structural stability, the inventors equipped the BabA protein with the C-terminal hexa-lysine-peptide. The hexa-lysine substitution improved the structural stability of the BabA protein as reflected in its much-improved ability for crystallization and, furthermore, its greatly increased Leb-binding affinity (as shown by ELISA).

This demonstrated, as a surprising effect, that the 6K-BabA antigen induced several-folds stronger IT50 responses that protected against gastric cancer and inflammation. Thus, structural stabilization of the vaccine antigen can be an essential key factor for vaccination efficacy and protection against development of gastric disease. The series of results suggest that structural stabilization of vaccine antigens by the hexa-lysine peptide modification can similarly improve the vaccination efficacy also of other vaccine antigens. Thus, hexa-lysine modified antigens might demonstrate general improvement in inducing protective antibody responses.

### Example 20. Evaluation of IT50 of Mice Sera Vaccinated with Hexa-Lysine-Modified BabA

Vaccination was performed on the following five experimental groups: BabA 6xLys 1 μg (mice sera 1-9, 19); BabA 1 μg (mice sera 10-18, 20); BabA 6xLys 0.2 μg (mice sera 21-30); BabA 0.2 μg (mice sera 33-41, 31); and Control (mice sera 42-47, 49-50, 32). IT50 was performed according to standard lab protocol (for example as presented in closer detail in Example 1).

The results are shown below (the table also presented in FIG. 8):

TABLE 1
IT50 of the mice sera from all experimental groups

| | Mice | | | | |
|---|---|---|---|---|---|
| Exp. group | sera | IT50 | Mean | Median | Notes |
| BabA 6xLys 1 μg | 1 | 13972 | 14787 | 13609 | |
| | 2 | 6712 | | | |
| | 3 | 7544 | | | |
| | 4 | 13246 | | | |
| | 5 | 16013 | | | |
| | 6 | 23036 | | | |
| | 7 | 36940 | | | |
| | 8 | 3659 | | | |
| | 9 | 20378 | | | |
| | 19 | 6366 | | | |
| BabA 1 μg | 10 | 5344 | 10401 | 8007 | |
| | 11 | 3187 | | | |
| | 12 | 23445 | | | |
| | 13 | 10331 | | | |
| | 14 | 7964 | | | |
| | 15 | 2295 | | | |
| | 16 | 8049 | | | |
| | 17 | 28379 | | | |
| | 18 | 8616 | | | |
| | 20 | 6399 | | | |
| BabA 6xLys 0.2 | 21 | 7486 | 949 | 87 | |
| μg | 22 | 56 | | | |
| | 23 | 86 | | | |
| | 24 | 88 | | | |
| | 25 | 29 | | | |
| | 26 | 47 | | | |
| | 27 | 1063 | | | |
| | 28 | 446 | | | |
| | 29 | 189 | | | |
| | 30 | 1 | | | |
| BabA 0.2 μg | 33 | 1443 | 175 | 22 | |
| | 34 | 1 | | | |
| | 35 | 186 | | | |
| | 36 | 23 | | | |
| | 37 | 45 | | | |
| | 38 | 22 | | | |
| | 39 | 1 | | | |
| | 40 | 24 | | | |
| | 41 | 8 | | | |
| | 31 | 1 | | | |
| Control | 42 | 1 | 3.5 | 1 | IT50 of pooled |
| | 43 | 1 | | | sera was |
| | 44 | 1 | | | negative |
| | 45 | 1 | | | |
| | 46 | 1 | | | |
| | 47 | 1 | | | |
| | 49 | 14 | | | |
| | 50 | 11 | | | |
| | 32 | 1 | | | |

IT50 was then determined for the 3^{rd} Vaccination; Vaccine-1 and Vaccine-2 and Vaccine-3 (with no adjuvant). The results are shown below (Results also presented in FIG. 8, Panel C).

TABLE 2
The mean and median values of IT50 against 17875

H. pylori 17875

| | Median | Mean |
|---|---|---|
| Group G1 | 7025 | 7567 |
| Group G2 | 3556 | 4224 |
| Group G3 | 1696 | 2509 |

TABLE 3
The IT50 values of the 3^{rd} vaccination, tested with
17875 (results also presented in FIG. 8, Panel C,
3^{rd} Vaccination; Vaccine-1(Pool G1) and Vaccine-2
(Pool G2)and Vaccine-3 (Pool G3 with no adjuvant)

| | |
|---|---|
| Pool G1-1 | 11709 |
| Pool G1-2 | 5823 |
| Pool G1-3 | 7787 |
| Pool G1-4 | 3882 |
| Pool G1-5 | 6263 |
| Pool G1-6 | 9941 |
| Pool G2-1 | 2517 |
| Pool G2-2 | 2828 |
| Pool G2-3 | 7679 |
| Pool G2-4 | 3645 |
| Pool G2-5 | 3469 |
| Pool G2-6 | 5207 |
| Pool G3-1 | 1124 |
| Pool G3-2 | 4679 |
| Pool G3-3 | 1094 |
| Pool G3-4 | 1696 |
| Pool G3-5 | 3953 |
| Human ELISA negative | 1 |
| serum | |
| Human ELISA positive serum | 158 |

### Example 21. The Vaccine Induced IT50s are Present in the Gastric Tissue

The inventors next tested for the presence of IT50 in the gastric tissue to investigate the functional significance of the sera IT50 blocking antibodies. The test showed that the IT50s of the stomach homogenates of the three vaccinated mice were ˜2 log-fold lower compared to the corresponding sera samples. However, the protein concentration in serum was ˜10-fold higher compared to the stomach extract. This follows, the IT50 titer/mg protein in the stomach tissue is merely ˜10-fold lower compared to sera (FIG. 9, Panel E). The new results argue that the sera IT50 titer is representative for the IT50 titer in the corresponding gastric tissue. The merely ˜10-fold difference suggest that the blocking antibodies are delivered to the stomach tissue by transudation of serum IgG, and once present in the stomach, they perform the local protection against gastric disease.

Without further elaboration, it is believed that a person skilled in the art can, using the present description, including the examples, utilize the present invention to its fullest extent. Also, although the invention has been described herein with regards to its preferred embodiments, which constitute the best mode presently known to the inventors, it should be understood that various changes and modifications as would be obvious to one having the ordinary skill in this art may be made without departing from the scope of the invention which is set forth in the claims appended hereto.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCES

Aro, P., Storskrubb, T., Ronkainen, J., Bolling-Sternevald, E., Engstrand, L., Vieth, M., Stolte, M., Talley, N. J., and Agreus, L. (2006). Peptic ulcer disease in a general adult population: the Kalixanda study: a random population-based study. Am J Epidemiol 163, 1025-1034. 10.1093/aje/kwj129
Aspholm, M., Kalia, A., Ruhl, S., Schedin, S., Arnqvist, A., Linden, S., Sjostrom, R., Gerhard, M., Semino-Mora, C., Dubois, A., et al. (2006). Helicobacter pylori adhesion to carbohydrates. Methods Enzymol 417, 293-339. 10.1016/S0076-6879 (06) 17020-2.
Aspholm-Hurtig, M., Dailide, G., Lahmann, M., Kalia, A., Ilver, D., Roche, N., Vikstrom, S., Sjostrom, R., Linden, S., Backstrom, A., et al. (2004). Functional adaptation of BabA, the H. pylori ABO blood group antigen binding adhesin. Science 305, 519-522. 10.1126/science.1098801
Atherton, J. C., Cullen, D. J., Kirk, G. E., Hawkey, C. J., and Spiller, R. C. (1996). Enhanced eradication of Helicobacter pylori by pre-versus post-prandial amoxycillin suspension with omeprazole: implications for antibiotic delivery. Aliment Pharmacol Ther 10, 631-635. 10.1046/j.1365-2036.1996.37179000.x
Boren, T., Falk, P., Roth, K. A., Larson, G., and Normark, S. (1993). Attachment of Helicobacter pylori to human gastric epithelium mediated by blood group antigens. Science 262, 1892-1895. 10.1126/science.8018146
Bugaytsova, J. A., Bjornham, O., Chernov, Y. A., Gideonsson, P., Henriksson, S., Mendez, M., Sjostrom, R., Mahdavi, J., Shevtsova, A., Ilver, D., et al. (2017). Helicobacter pylori Adapts to Chronic Infection and Gastric Disease via pH-Responsive BabA-Mediated Adherence. Cell Host Microbe 21, 376-389. 10.1016/j.chom.2017.02.013
Eriksson, A. M., Schön, K. M. & Lycke, N. Y. The cholera toxin-derived CTA1-DD vaccine adjuvant administered intranasally does not cause inflammation or accumulate in the nervous tissues. J. Immunol. 173, 3310-3319 (2004).
Falk, P. G., Bry, L., Holgersson, J., and Gordon, J. I. (1995). Expression of a human alpha-1,3/4-fucosyltransferase in the pit cell lineage of FVB/N mouse stomach results in production of Leb-containing glycoconjugates: a potential transgenic mouse model for studying Helicobacter pylori infection. Proc Natl Acad Sci USA 92, 1515-1519. 10.1073/pnas.92.5.1515
Fei, Y. Y., Schmidt, A., Bylund, G., Johansson, D. X., Henriksson, S., Lebrilla, C., Solnick, J. V., Boren, T., and Zhu, X. D. (2011). Use of real-time, label-free analysis in revealing low-affinity binding to blood group antigens by Helicobacter pylori. Anal Chem 83, 6336-6341. 10.1021/ac201260c
Fox, J. G., and Wang, T. C. (2007). Inflammation, atrophy, and gastric cancer. J Clin Invest 117, 60-69. 10.1172/JCI30111
Gerhard, M., Lehn, N., Neumayer, N., Boren, T., Rad, R., Schepp, W., Miehlke, S., Classen, M., and Prinz, C. (1999). Clinical relevance of the Helicobacter pylori gene for blood-group antigen-binding adhesin. Proc Natl Acad Sci USA 96, 12778-12783. 10.1073/pnas.96.22.12778
Hage N, Renshaw JG, Winkler GS, Gellert P, Stolnik S, Falcone FH., Improved expression and purification of the Helicobacter pylori adhesin BabA through the incorporation of a hexa-lysine tag. Protein Expr Purif. 2015 February; 106:25-30. doi: 10.1016/j.pep.2014.10.009. Epub 2014 Oct. 25.PMID: 25448827)
Hansen, L. M., Gideonsson, P., Canfield, D. R., Borén, T., and Solnick, J. V. (2017). Dynamic Expression of the BabA Adhesin and Its BabB Paralog during Helicobacter pylori Infection in Rhesus Macaques. Infect Immun 85. 10.1128/iai.00094-17
Kamada, N., Sakamoto, K., Seo, S. U., Zeng, M. Y., Kim, Y. G., Cascalho, M., Vallance, B. A., Puente, J. L., and Nunez, G. (2015). Humoral Immunity in the Gut Selectively Targets Phenotypically Virulent Attaching-and-Effacing Bacteria for Intraluminal Elimination. Cell Host Microbe 17, 617-627. 10.1016/j.chom.2015.04.001
Kato et al., Mutational analysis of protein solubility enhancement using short peptide tags. Biopolymers 85, 12-18 (2007)
Lycke N & Schön K, The B cell targeted adjuvant, CTA1-DD, exhibits potent mucosal immunoenhancing activity despite pre-existing anti-toxin immunity, Vaccine. 2001 Mar. 21; 19 (17-19): 2542-8. doi: 10.1016/s0264-410x (00) 00487-4.
Moonens, K., Gideonsson, P., Subedi, S., Bugaytsova, J., Romao, E., Mendez, M., Norden, J., Fallah, M., Rakhimova, L., Shevtsova, A., et al. (2016). Structural Insights into Polymorphic ABO Glycan Binding by Helicobacter pylori. Cell Host Microbe 19, 55-66. 10.1016/j.chom.2015.12.004.
Paraskevopoulou V, Artiaga VG, Rowlinson R, Winkler GS, Gellert P, Stolnik S, Overman R, Falcone FH The hexa lysine peptide has also been suggested to increase thermal stability of the recombinant protein (Introduction of a C-terminal hexa-lysine tag increases thermal stability of the LacDiNac binding adhesin (LabA) exodomain from Helicobacter pylori. Protein Expr Purif. 2019 November; 163:105446. doi: 10.1016/j.pep.2019.105446. Epub 2019 Jul. 1.PMID: 31271862).
Paraskevopoulou V, Alissa M, Hage N, Falcone FH, Introduction of a Hexalysine (6 K) Tag Can Protect from N-Terminal Cleavage and Increase Yield of Recombinant Proteins Expressed in the Periplasm of E. coli, Methods Mol Biol. 2022; 2406:155-167. doi: 10.1007/978-1-0716-1859-2_9.PMID: 35089556
Parsonnet, J., Friedman, G. D., Vandersteen, D. P., Chang, Y., Vogelman, J. H., Orentreich, N., and Sibley, R. K. (1991). Helicobacter pylori infection and the risk of gastric carcinoma. N Engl J Med 325, 1127-1131. 10.1056/NEJM199110173251603
Salama, N. R., Hartung, M. L., and Muller, A. (2013). Life in the human stomach: persistence strategies of the bacterial pathogen Helicobacter pylori. Nat Rev Microbiol 11, 385-399. 10.1038/nrmicro3016
Schreiber, S., Konradt, M., Groll, C., Scheid, P., Hanauer, G., Werling, H. O., Josenhans, C., and Suerbaum, S. (2004). The spatial orientation of Helicobacter pylori in the gastric mucus. Proc Natl Acad Sci USA 101, 5024-5029. 10.1073/pnas.0308386101
Sutton, P., and Boag, J. M. (2019). Status of vaccine research and development for Helicobacter pylori. Vaccine 37, 7295-7299. 10.1016/j.vaccine.2018.01.001.
Ågren, L., Löwenadler, B. & Lycke, N. A novel concept in mucosal adjuvanticity: the CTA1-DD adjuvant is a B cell-targeted fusion protein that incorporates the enzymatically active cholera toxin A1 subunit. Immunol. Cell Biol. 76, 280-287 (1998).

1-33. (canceled)

34. A method for preventing, alleviating and/or treating a gastric disease in a subject diagnosed with H. pylori infection, comprising administering to the subject an effective amount of a vaccine composition containing an antigen wherein the administration of said antigen results in the induction of an antibody response that targets a conserved structural epitope in the Carbohydrate Binding Domain (CBD) of the ABO/Leb-Blood group Antigen-Binding Adhesin (BabA), sufficient to reduce mucosal attachment of H. pylori without eradicating the H. pylori infection in said subject,
wherein said gastric disease is chosen from gastroesophageal reflux disease (GERD), chronic active gastritis (GA), peptic ulcer disease (DU), gastric ulcer disease, and gastric cancer;
wherein the antigen is a recombinant modified BabA exhibiting conserved structural epitopes including four disulfide bonds whereof the second disulfide bond spans seven amino acids between two adjacent cystein molecules, having an amino acid sequence with at least 80% identity, such as at least 85% identity, such as at least 90% identity, or at least 95% identity with the sequence defined by SEQ ID NO. 2; and
wherein the composition is formulated as a vaccine comprising a pharmaceutically acceptable carrier and an adjuvant chosen from mucosal or systemic adjuvants that preferentially activate the humoral immune responses with induction of protective antibodies.

35. The method according to claim 34, wherein the gastric disease is gastric cancer and wherein preventing gastric cancer is interpreted the reduction of the incidence of gastric cancer in a population infected with H. pylori or susceptible to being infected by H. pylori, compared to the incidence in a control population not receiving said composition.

36. A method for preventing, alleviating and/or treating a gastric disease in a subject diagnosed with H. pylori infection, comprising administering to the subject an effective amount of a vaccine composition containing an antigen wherein the administration of said antigen results in the induction of an antibody response that targets a conserved structural epitope in the Carbohydrate Binding Domain (CBD) of the ABO/Leb-Blood group Antigen-Binding Adhesin (BabA), sufficient to reduce mucosal attachment of H. pylori without eradicating the H. pylori infection in said subject;
wherein said gastric disease is chosen from gastroesophageal reflux disease (GERD), chronic active gastritis (GA), peptic ulcer disease (DU), gastric ulcer disease, and gastric cancer;
wherein the antigen is a recombinant modified BabA exhibiting conserved structural epitopes including four disulfide bonds whereof the second disulfide bond spans seven amino acids between two adjacent cystein molecules, having an amino acid sequence with at least 80% identity, such as at least 85% identity, such as at least 90% identity, or at least 95% identity with the sequence defined by SEQ ID NO. 2;
wherein the composition is formulated as a vaccine comprising a pharmaceutically acceptable carrier and an adjuvant chosen from mucosal or systemic adjuvants that preferentially activate the humoral immune responses with induction of protective antibodies; and
wherein the antigen is structurally stabilized by the incorporation of a continuous sequence of at least four positive amino acids is included in said sequence, preferably at the C-terminal of said sequence.

37. The method according to claim 36, wherein said continuous sequence of at least four positive amino acid molecules is a sequence of at least four lysine molecules.

38. The method according to claim 36, wherein said continuous sequence of at least four positive amino acid molecules is a sequence of at least four arginine molecules.

39. The method according to claim 36, wherein said continuous sequence of at least four positive amino acid molecules is a sequence of at least six lysine molecules.

40. The method according to claim 36, wherein the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

41. The method according to claim 34, wherein the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

42. The method according to claim 34, wherein said adjuvant is an aluminium-based adjuvant chosen from aluminium hydroxide, aluminium phosphate, and aluminium hydroxyphosphate sulfate.

43. The method according to claim 34, wherein said adjuvant is a cholera toxin-derived adjuvant, more preferably CTA1-DD.

44. A vaccine formulation comprising an immunogenically effective amount of an antigen for use in a method of preventing or treating gastric disease in a subject diagnosed with Helicobacter pylori (H. pylori) infection,
wherein said antigen is capable of inducing an antibody response sufficient to reduce mucosal attachment of H. pylori without eradicating the H. pylori infection;
wherein said antibody response specifically targets a conserved structural epitope in the Carbohydrate Binding Domain (CBD) of the ABO/Leb-Blood group Antigen-Binding Adhesin (BabA) of H. pylori;
wherein said antigen is a recombinant modified BabA exhibiting conserved structural epitopes including four disulfide bonds whereof the second disulfide bond spans seven amino acids between two adjacent cystein molecules, having an amino acid sequence with at least 80% identity, such as at least 85% identity, such as at least 90% identity, or at least 95% identity with the sequence defined by SEQ ID NO. 2;
wherein said gastric disease is chosen from gastroesophageal reflux disease (GERD), chronic active gastritis (GA), peptic ulcer disease (DU), gastric ulcer disease, and gastric cancer, and
wherein the composition comprises a pharmaceutically acceptable carrier and an adjuvant chosen from mucosal or systemic adjuvants that preferentially activate the humoral immune responses with induction of protective antibodies.

45. The vaccine formulation according to claim 44, wherein the disease is gastric cancer, and wherein preventing gastric cancer is interpreted as the reduction of the incidence of gastric cancer in a population infected with H. pylori or susceptible to being infected by H. pylori, compared to the incidence in a control population not receiving said composition.

46. The vaccine formulation according to claim 44, wherein the antigen is structurally stabilized by the incorporation of a continuous sequence of at least four positive amino acids in its sequence, preferably at the C-terminal of said sequence.

47. The vaccine formulation according to claim 46, wherein said continuous sequence of at least four positive amino acid molecules is a sequence of at least four lysine molecules.

48. The vaccine formulation according to claim 46, wherein said continuous sequence of at least four positive amino acid molecules is a sequence of at least four arginine molecules.

49. The vaccine formulation according to claim 46, wherein said continuous sequence of at least four positive amino acid molecules is a sequence of at least six lysine molecules.

50. The vaccine formulation according to claim 46, wherein the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

51. The vaccine formulation according to claim 44, wherein the antigen is capable of inducing broadly blocking IgG antibodies (bbAbs) in serum or plasma corresponding to a 50% inhibition titer (IT50) value of at least 50 after mucosal, such as nasal, sublingual, intraoral, and peroral administration to a subject; or via systemic, such as subcutaneous or intramuscular administration to a subject.

52. The vaccine formulation according to claim 44, wherein said adjuvant is an aluminium-based adjuvant chosen from aluminium hydroxide, aluminium phosphate, and aluminium hydroxyphosphate sulfate.

53. The vaccine formulation according to claim 44, wherein said adjuvant is a cholera toxin-derived adjuvant, more preferably CTA1-DD.
